(19) United States
(12) Patent Application Publication
(10) Pub. No.: US 2021/0275589 A1
(43) Pub. Date: Sep. 9, 2021

(54) CO-RECEPTOR SYSTEMS FOR TREATING INFECTIOUS DISEASES

(71) Applicant: Nanjing Legend Biotech Co. Ltd., Nanjing, Jiangsu (CN)

(72) Inventors: Ming Zeng, Irving, TX (US); Lu Chen, Nanjing (CN); Shu Wu, Nanjing (CN); Lili Chen, Huzhou (CN); Xun Liu, Nanjing (CN)

(21) Appl. No.: 17/258,245

(22) PCT Filed: Jul. 12, 2019
(86) PCT No.: PCT/CN2019/095738

§ 371 Date: Jan. 6, 2021

(30) Foreign Application Priority Data
Jul. 13, 2018 (CN) PCT/CN2018/095650
May 16, 2019 (CN) PCT/CN2019/087259

(51) Int. Cl. A61K 35/17; C07K 16/28; C12N 5/0783

(52) U.S. Cl. CPC A61K 35/17; C07K 16/2812; C07K 16/2866; C07K 2319/03; C07K 2317/622; C07K 2319/02; C12N 5/0636

(57) ABSTRACT

Immune cells (such as T cells) comprising a chimeric receptor (CR), a chimeric co-receptor (CCOR), and/or a co-receptor (COR) are provided.

### RELATED APPLICATIONS

The patent application claims priority benefit to PCT/CN2018/095650, filed on Jul. 13, 2018, and PCT/CN2019/087259, filed on May 16, 2019, the content of each of which are incorporated herein by reference in their entirety.

### FIELD OF THE INVENTION

The invention relates to immune cells (such as T cells) comprising one or more engineered receptors useful for treating infectious diseases such as HIV.

### BACKGROUND OF THE INVENTION

T-cell mediated immunity is an adaptive process of developing antigen (Ag)—specific T lymphocytes to eliminate viruses, bacterial, parasitic infections or malignant cells.

Upon exposure to an Ag, a naïve T cell will mature into an activated effector T cell. This process occurs through the presentation of the Ag on the surface of an antigen presenting cell (APC). In particular, antigenic peptides from the Ag (i.e., the virus, bacterium, parasite, or malignant cell), are presented to T cells as part of the Major Histocompatibility Complex (MHC) located on the surface of APCs. The T cell receptor (TCR), found on the surface of T cells, is responsible for binding to the MHC and recognizing the bound peptides. This process triggers a signal transduction resulting in T cell activation and downstream immunological response.

In addition to the TCR, T cells harbor other cell surface receptors and markers that play a role in their functional characterization. For example, T cells displaying CD8 (CD8+ T cells, also known as cytotoxic or killer T cells) are responsible for targeting and destroying cells that are malignant, that are infected with viruses, or that display other signs of damage. T cells displaying CD4 (CD4+ T cells, or helper T cells) are characterized as generally aiding in the function of other immune cells. There are several further subsets of helper T cells, including Th1, Th2, and Th17. CD8+ and CD4+ T cells play essential roles in antigen response and T cell mediated immunity, but are not the only T cells in the immune system: there are various other classes including regulatory T cells (Tregs) and natural killer T (NKT) cells.

CD4+ T cells play perhaps the most important coordinating role in the immune system, having a central role in both T cell mediated immunity and B cell mediated, or humoral, immunity. In T cell mediated immunity, they play a role in the activation and maturation of CD8+ T cells. In B cell mediated immunity, they are responsible for stimulating B cells to proliferate and to induce B cell antibody class switching.

The central role CD4+ T cells play is perhaps best illustrated by the aftermath of an infection with human immunodeficiency virus (HIV). The virus is a retrovirus, meaning it carries its genetic information as RNA along with a reverse transcriptase enzyme that allows for the production of DNA from its RNA genome once it has entered a host cell. The DNA can then be incorporated into affected host cells, at which point the viral genes are transcribed and more viral particles are produced and released by the infected cell.

HIV preferentially targets CD4+ T cells; as a result, an infected patient's immune system becomes increasingly compromised, as the population of the main coordinating cell of the immune system is decimated. In fact, the progression of HIV to acquired immunodeficiency syndrome (AIDS) is marked by the patient's CD4+ T cell count. This targeting of CD4+ T cells by the virus is also what results in the inability of infected patients to successfully mount productive immune responses against various pathogens, including opportunistic pathogens.

The first treatment against HIV, zidovudine (ZDV) or azidothymidine (AZT), was approved by the United States Food and Drug Administration (FDA) in 1987. This drug was classified as a nucleoside reverse transcriptase inhibitor (NRTI). Over time, the initial success rates of the drug dropped, as viral mutations resulted in the inability of the drug to continue to suppress infection. Highly active antiretroviral therapy (HAART) was subsequently introduced to replace the initial antiviral therapy (ART).

By the mid-1990s, researchers and clinicians observed a benefit of combination treatment that included both NRTIs and protease inhibitors. Combination therapy continues to be in use today, and there are many combination therapies available to patients. In general, a combination therapy consists of two NRTIs plus a non-nucleoside reverse transcriptase inhibitor (NNRIT), a protease inhibitor, or an integrase inhibitor. In sum, there are currently six classes of drugs that are used as a part of combination therapies: entry inhibitors, nucleoside reverse transcriptase inhibitors, nucleotide reverse transcriptase inhibitors, non-nucleoside reverse transcriptase inhibitors, protease inhibitors, and integrase inhibitors.

Targeting the virus with various pharmacological classes of drugs prevents viral resistance and has shown a significant efficacy in infected patients, but requires high levels of adherence by patients to ensure its complete efficacy. In fact, non-adherence can result in the emergence of drug-resistant strains, leading to further difficulties in effectively managing and treating both the disease and subsequent complications in patients.

The disclosures of all publications, patents, patent applications and published patent applications referred to herein are hereby incorporated herein by reference in their entirety.

### BRIEF SUMMARY OF THE INVENTION

The present application in one aspect provides an engineered immune cell comprising a chimeric receptor (CR) comprising a CR antigen binding domain specifically recognizing a CR target antigen, a CR transmembrane domain, and an intracellular CR signaling domain; and a chimeric co-receptor (CCOR) comprising a CCOR antigen binding domain specifically recognizing a CCOR target antigen, a CCOR transmembrane domain, and an intracellular CCOR co-stimulatory domain. In some embodiments, the CR target antigen is CCR5 or CXCR4 and the CCOR target antigen is CD4. In some embodiments, the CR target antigen is CD4 and the CCOR target antigen is CCR5 or CXCR4. In some embodiments, the engineered immune cell further comprises one or more co-receptors (CORs).

In one aspect, the invention provides an engineered immune cell comprising a CR comprising a CR antigen binding domain specifically recognizing a CR target antigen, a CR transmembrane domain, and an intracellular CR signaling domain. In some embodiments, the CR target antigen is selected from the group consisting of CCR5, CXCR4, and CD4. In other embodiments, the engineered immune cell further comprises one or more CORs. In some embodiments, the CR antigen binding domain comprises at least two of a CD4 binding moiety, a CCR5 binding moiety, and an anti-HIV antibody moiety (such as a broadly neutralizing antibody (“bNAb”)) moiety. In some embodiments, the CR is a tandem CR comprising a CD4 binding moiety and a CCR5 binding moiety. In some embodiments, the CR is a tandem CR comprising a CD4 binding moiety and an anti-HIV antibody moiety (such as a bNAb moiety). In some embodiments, the CR is a tandem CR comprising a CCR5 binding moiety and an anti-HIV antibody moiety (such as a bNAb moiety). The CD4 moiety, CCR5 moiety, and/or bNAb moiety may be linked to each other directly or via a linker.

In some embodiments, the invention provides an engineered immune cell comprising a first nucleic acid encoding a CR, wherein the CR comprises a CR antigen binding domain specifically recognizing a CR target antigen, a CR transmembrane domain, and an intracellular CR signaling domain, and a second nucleic acid encoding a CCOR, wherein the CCOR comprises a CCOR antigen binding domain specifically recognizing a CCOR target antigen, a CCOR transmembrane domain, and an intracellular CCOR co-stimulatory signaling domain. In some embodiments, the CR target antigen is CCR5 or CXCR4 and the CCOR target antigen is CD4. In some embodiments, the CR target antigen is CD4 and the CCOR target antigen is CCR5 or CXCR4. In some embodiments, the immune cell further comprises one or more nucleic acid(s) encoding one or more CORs.

In some embodiments, the invention provides an engineered immune cell comprising a nucleic acid encoding a CR, wherein the CR comprises a CR antigen binding domain specifically recognizing a CR target antigen, a CR transmembrane domain, and an intracellular CR signaling domain. In some embodiments, the CR target antigen is selected from the group consisting of CCR5, CXCR4, and CD4. In some embodiments, the immune cell further comprises one or more nucleic acid(s) encoding one or more CORs.

In some embodiments according to some or more of the above embodiments, the CR further comprises an intracellular CR co-stimulatory domains. In other embodiments, the CR does not comprise an intracellular co-stimulatory domain.

In some embodiments according to one or more of the above embodiments, the nucleic acid encoding the CR is under an inducible promoter. In other embodiments, the nucleic acid encoding the CR is constitutively expressed.

In some embodiments according to any one or more of the above embodiments, the nucleic acid encoding the CCOR and/ or COR is under an inducible promoter. In other embodiments, the nucleic acid encoding the CCOR and/ or COR is constitutively expressed. In yet other embodiments, the nucleic acid encoding the CCOR and/ or COR is inducible upon activation of the immune cell.

In some embodiments, the first nucleic acid and the second nucleic acid are on the same vector. In some embodiments, the first nucleic acid and the second nucleic acid are under the control of the same promoter. In other embodiments, the first nucleic acid and the second nucleic acid are on different vectors.

In some embodiments, one or more COR-encoding nucleic acids are on the same vector as the first nucleic acid. In some embodiments, one or more COR-encoding nucleic acids are on the same vector as the second nucleic acid. In some embodiments, the one or more COR-encoding nucleic acid and the first nucleic acid or the second nucleic acid is under the control of the same promoter.

In some embodiments according to any of the above embodiments, the CR target antigen is CCR5 or CXCR4 and the CCOR target antigen is CD4. In other embodiments, the CR target antigen is CD4 and the CCOR target antigen is CCR5 or CXCR4.

In some embodiments according to any of the above embodiments, the one or more COR is selected from the group consisting of CXCR5, α4β7, and CXCR9. In some embodiments, at least one of the one or more COR is CXCR5. In other embodiments, at least one of the one or more COR is α4β7. In yet other embodiments, at least one of the one or more COR is CCR9. In yet further embodiments, one or more COR comprises both α4β7 and CCR9.

In some embodiments according to any of the above embodiments, the immune cell is modified to reduce or eliminate expression of CCR5 within the cell. In some embodiments, the CCR5 gene is inactivated by using the method selected from the group consisting of: CRISPR/Cas9, TALEN ZFN, siRNA, and antisense RNA.

In some embodiments according to any of the above embodiments, the CR antigen binding domain is selected from the group consisting of Fab, a Fab′, a (Fab′)₂, an Fv, a single chain Fc (scFv), a single domain antibody (sdAb), and a peptide ligand specifically binding to the CR target antigen. In some embodiments, the CR antigen binding domain is scFv or sdAb.

In some embodiments according to any of the above embodiments, the CCOR antigen binding domain is selected from the group consisting of Fab, a Fab′, a (Fab′)₂, an Fc, a single chain Fv (scFv), a single domain antibody (sdAb), and a peptide ligand specifically binding to the CCOR target antigen. In some embodiments, the CCOR antigen binding domain is scFv or sdAb.

In some embodiments according to any of the above embodiments, the intracellular CR signaling domain is selected from the group consisting of CD3ζ, FcRγ, FcRβ, CD3γ, CD3δ, CD3ε, CD5, CD22, CD79a, CD79b, and CD66d.

In some embodiments according to one or more of the above embodiments, the CR or CCOR co-stimulatory domain is selected from the group consisting of a co-stimulatory domain of one or more of CD28, 4-1BB (CD137), CD27, OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, and a ligand that specifically binds with CD83.

In some embodiments according to any of the above embodiments, the engineered immune cell is selected from the group consisting of T cells, B cells, NK cells, dendritic cells, eosinophils, macrophages, lymphoid cells, and mast cells. In some embodiments, the engineered immune cell is selected from a cytotoxic T cell, a helper T cell, and a natural killer T cell. In some embodiments, the engineered immune cell is a cytotoxic T cell.

In some embodiments, the invention provides a pharmaceutical composition comprising an engineered immune cell of any of the embodiments above and a pharmaceutically acceptable carrier. In some embodiments, the pharmaceutical composition comprises at least two different types of engineered immune cells.

In some embodiments, the invention provides a method of treating an infectious disease in an individual, comprising administering to the individual an effective amount of a pharmaceutical composition described above. In some embodiments, the infectious disease is an infection by a virus selected from the group of HIV and HTLV. In some embodiments, the infectious disease is HIV.

In some embodiments of the above method, the individual is a human.

In some embodiments, the invention also provides a method of making an engineered immune cell comprising providing a population of immune cells and introducing into the population of immune cells a first nucleic acid encoding the CR.

In some embodiments, a second nucleic acid encoding the CCOR is introduced into the population of immune cells. In some embodiments, the first nucleic acid and the second nucleic acid are introduced to the cells simultaneously. In other embodiments, the first nucleic acid and the second nucleic acid are introduced into the cells sequentially. In some embodiments, one or more nucleic acids encoding one or more CORs are introduced into the population of immune cells. In some embodiments, the first nucleic acid and the second nucleic acid and/ or the COR encoding nucleic acids are introduced into the cell via a viral vector.

In some embodiments, the method of making the engineered immune cell further comprises inactivating the CCR5 gene in the cell. In some embodiments, the CCR5 gene is inactivated by using the method selected from the group consisting of: CRISPR/Cas9, TALEN ZFN, siRNA, and antisense RNA. In some embodiments, the population of immune cells is obtained from the peripheral blood of an individual. In some embodiments, the population of immune cells is further enriched for CD4+ cells. In other embodiments, the population of immune cells is further enriched for CD8+ cells.

All references cited herein, including patent applications and publications, are incorporated by reference in their entirety.

### BRIEF DESCRIPTION OF THE DRAWINGS

FIG. 1A and FIG. 1B show schematic representations of several exemplary receptor molecules having different antigen-binding domains, including anti-CCR5/CXCR4 and anti-CD4.

FIG. 2A and FIG. 2B show schematic representations of exemplary general antigen (FIG. 2B) and specific antigen (HIV) (FIG. 2A) targeting constructs plus CXCR5 expression.

FIGS. 3A and 3B show schematic representation of exemplary general antigen (FIG. 3B) and specific antigen (HIV) (FIG. 3A) targeting constructs plus CCR9 and α4β7.

FIG. 4 shows a schematic representation of exemplary HIV targeting constructs plus CCR5 gene knockout.

FIG. 5 shows a schematic representation of exemplary HIV targeting constructs plus CCR9 and α4β7 plus CXCR5 plus CCR5 gene knockout.

FIG. 6A and FIG. 6B show schematic representations of exemplary CAR constructs containing anti-CD4 or anti-CCR5 or anti-CXCR4 scFv or sdAb (FIG. 6A) or anti-CD4 and anti-CCR5 scFv or sdAb linked in tandem (FIG. 6B). FIG. 6C shows a schematic representation of exemplary CAR constructs containing anti-CD4 or anti-CCR5 scFv or sdAb tandemly linked to a broadly neutralizing antibody that recognizes HIV.

FIG. 7 shows the in vitro screening result of CAR-Ts bearing different scFv sequences. FIG. 7A shows the relative target-killing effect of 14 anti-CCR5 CAR-Ts. FIG. 7B shows the relative target-killing effect of 16 anti-CD4 CAR-Ts. FIG. 7C shows the relative target-killing effect of 8 anti-CXCR4 cells.

FIGS. 8A-8E show CAR expression of various constructs. 8A: CAR expression on anti-CD4-CART No.13 cells compared with un-transduced T cells; FIG. 8B: CAR expression on anti-CCR5-CAR T No.13 cells compared with un-transduced T cells; FIG. 8C: CAR expression on anti-CD4-CAR T cells expressing CXCR5. FIG. 8D: CAR expression on anti-CCR5-CAR T cells expressing CXCR5; FIG. 8E: CAR expression on anti-CD4/anti-CCR5 tandem CAR T cells, anti-CD4/anti-CCR5 tandem CAR-CXCR5 T cells, anti-CD4/anti-CCR5 tandem CAR-CXCR5-C34 T cells.

FIG. 9 shows proliferation of anti-CD4 CART No. 13 cells in vitro. 5×10⁵ cells were transduced with the CAR lentiviruses at day 0. Cells were enumerated at day 4, 6, and 10 after transduction.

FIGS. 10A-F shows cytotoxic effects of various CAR-T cells towards target cells. CFSE labeled pan T cells were used as target cells. FIG. 10A: anti-CD4 CART No. 13 cells; FIG. 10B: anti-CCR5 CART No. 13 cells; FIG. 10C: anti-CD4 CART cells expressing CXCR5; FIG. 10D: anti-CCR5CAR T cells expressing CXCR5; FIG. 10E: anti-CD4-anti-CCR5 tandem CART cells; FIG. 10F: comparison between anti-CD4-anti-CCR5 tandem CAR T cells and anti-CD4/anti-CCR5 tandem CAR-CXCR5-C34 T cells.

FIG. 11 shows expression of cytokines by anti-CD4 CART No. 13 cells. Effector anti-CD4 CART No. 13 cells were co-cultured with target cells at 2:1 and 0.5:1 ratio for 24 hours. The supernatant from cell cultures was collected and the cytokine levels in the supernatant were detected by Homogeneous Time Resolved Fluorescence (HTRF) assay.

FIG. 12A and FIG. 12B show schematic representations of exemplary eTCRs containing anti-CD4 or anti-CCR5 scFv or sdAb (FIG. 12A), or anti-CD4 and anti-CCR5 scFv or sdAb linked in tandem (FIG. 12B). FIG. 12C shows the relative target cell killing capability of CD4 No.13 CAR-T, CD4 eTCR, CD4 eTCR No. 11. FIG. 12D shows the relative target cell killing capability of several CCR5 eTCR cells.

FIG. 13 show results of eTCR T cell characterizations. FIG. 13A shows detection of transduced gene expression on anti-CD4 eTCR-T and anti-CCR5 eTCR-T cells. FIG. 13B shows expression of cytokines by anti-CD4 eTCR-T T cells. Effector anti-CD4 eTCR T cells were co-cultured with target cells at 2:1 and 0.5:1 ratio for 24 hours. The supernatant from cell cultures were collected and the cytokine levels in the supernatant were detected by HTRF assay. FIG. 13C shows the expansion of anti-CD4 eTCR-T cells in vitro. 5×10⁵ cells were transduced with the eTCR lenti-viruses at day 0. Cells were enumerated at day 4, 6, and 10 post transduction.

FIG. 14A and FIG. 14B. Shows the cytotoxic effect of anti-CD4 and anti-CCR5 eTCR-T cells. Anti-CD4 eTCR T cells, anti-CCR5 eTCR T cells or control UnT cells were co-cultured with CFSE labeled primary T cells at 2:1 ratio for 24 hours. CD4+% (A) or CCR5+% (B) in CFSE+ cells was recorded by flow cytometry.

FIGS. 15A-15D show schematic representations of exemplary CARS or eTCRs containing anti-CD4 or anti-CCR5 scFv or sdAb (FIGS. 15A and 15C), or anti-CD4 and anti-CCR5 scFv or sdAb linked in tandem (FIGS. 15B and 15D). The CAR T cells or eTCR T cells further express CXCR5.

FIGS. 16A and 16B show expression of CXCR5 on the CD4-CART-CXCR5 cells and CCR5-CART-CXCR5 cells.

FIGS. 17A-D show schematic representations of exemplary CARs or eTCRs containing anti-CD4 or anti-CCR5 scFv or sdAb (FIGS. 17A and 17C), or anti-CD4 and anti-CCR5 scFv or sdAb linked in tandem (FIGS. 17B and 17D). The CAR T cells or eTCR T cells further express CXCR5 and a broadly neutralizing antibody.

FIGS. 18A and 18B show the effect of anti-CD4 CAR T cells on controlling viral load. FIG. 18A: anti-CD4 CAR T No. 13 cells were co-cultured with virus-free target cells or HIV pseudovirus infected target cells at 1:1 ratio for 24 hours. Anti-CD19 CAR-T (SEQ ID NO. 77) cells were used as control CAR-T. The remaining amount of target cells were detected by real-time PCR. FIG. 18B: anti-CD4 CAR T No.13 cells or untransduced T cells were co-cultured with EGFP+ pseudo-infected target cells at indicated ratio for 24 hours. EGFP+ target cells were detected by flow cytometry.

FIG. 19 shows the CAR-T effect on controlling Simian/Human Immunodeficiency Virus (SHIV) infection. Rhesus macaque CD4+ T cells were purified from the monkey peripheral blood and were activated with anti-CD3/CD28 beads for 4 days before they were challenged with the SHIV_{SF162P3}. SHIV infected cells were used as target cells and were cocultured with anti-CD4 CART No.13, anti-CCR5 CART No. 13 and tandem anti-CD4/anti-CCR5 CAR-T cells for 3 days. FIG. 19A shows the presence of viral p27 antigen by intracellular staining. FIG. 19B shows the viral RNA level in the cell culture supernatant and the integrated DNA level in the genomic DNA.

FIGS. 20A and 20B show anti-CD4 CAR T No.13 cells killed T cell lymphoma cell lines (SupT1 and HH) in a dose dependent manner.

FIGS. 21A and 21B show in vivo efficacy of anti-CD4 CAR T No. 13 cells. CDX mice were separated into 3 groups: group 1 mice received HBSS, group 2 mice received control unT cells, and group 3 mice received anti-CD4 CAR T No. 13 treatment. FIG. 21A shows the tumor volume. FIG. 21B shows mice body weight after treatment.

FIG. 22A show expression of split signal CAR constructs on the T cell surface. In ssCCR5CD4 CAR T cells, an anti-CCR5 moiety was linked to an HA tag and a CD3ζ intracellular domain, and anti-CD4 moiety was linked a Myc tag and an intracellular co-stimulatory domain. In ssCD4CCR5 CAR T cells, an anti-CD4 moiety was linked to an HA tag and a CD3ζ intracellular domain, and anti-CCR5 moiety was linked a Myc tag and an intracellular co-stimulatory domain. The two moieties were linked by a P2A. UNT represents un-transduced T cells. The expression of Myc tag was shown in this figure as a representative for the expression of the split signal CAR system. FIG. 22B shows the cytotoxic effect of the constructs. CAR T cells or UNT cells were co-cultured with CFSE labeled target cells at 0.5:1 ratio for 24 hours.

FIGS. 23A-23C shows in vivo effects of the CAR T therapy. FIG. 23A shows the percentage of CCR5+ cells at different time points in HIS mouse treated with anti-CCR5 CAR T No.13 cells. FIG. 23B shows the percentage of CCR5+ cells in HIS mouse treated with tandem anti-CD4 anti-CCR5 CAR T cells. FIG. 23C shows the percentage of CD4+ cells in HIS mouse treated with tandem anti-CD4 anti-CCR5 CAR T cells.

### DETAILED DESCRIPTION OF THE INVENTION

The present application provides immune cells (such as T cells) expressing a chimeric receptor (“CR”) that specifically recognizes a CR target antigen selected from any one of CCR5 (or CXCR4) and CD4 and an intracellular CR signaling domain capable of activating the immune cells. In some embodiments when the CR does not contain a co-stimulatory domain, the CR can be co-expressed with a chimeric co-receptor (“CCOR”) which contains a CCOR co-stimulatory domain and specifically recognizes the other one of CCR5 (or CXCR4) or CD4 (CCOR target antigen). The CCOR thus provides the requisite co-stimulation upon binding of the CCOR target antigen, ensuring that the immune cell is only activated when both the CR target antigen and CCOR target antigen are present and recognized by the immune cell. The immune cell can further express one or more co-receptors (“COR”), for example co-receptors that facilitate migration of the immune cells to a desired location, such as follicles (a CXCR5 receptor). and gut (a α4β7 receptor or CCR9 receptor). In addition, the immune cell can be further modified to reduce or knockout the expression of the CCR5 receptor, thus increasing the resistance of the immune cells (such as CD4+ immune cells) to viral infection.

Thus, the present invention in one aspect provides an immune cell comprising a CR and a CCOR. In another aspect, there is provided an immune cell comprising a CR and a COR. In some embodiments, the immune cell comprises a CR, a CCOR, and one or more CORs. In some embodiments, the immune cell is further modified to reduce or knockout the expression of CCR5.

Also provided are nucleic acid systems expressing the CR, CCOR, and/or COR in immune cells.

Also provided are methods of making and using the engineered immune cells for treatment purposes, as well as kits and articles of manufacture useful for such methods.

### Definitions

The term “antibody” herein is used in the broadest sense and specifically covers monoclonal antibodies (including full length monoclonal antibodies), multispecific antibodies (e.g., bispecific antibodies), and antibody fragments so long as they exhibit the desired biological activity.

The terms “native antibody”, “full length antibody,” “intact antibody” and “whole antibody” are used herein interchangeably to refer to an antibody in its substantially intact form, not antibody fragments as defined below. The terms particularly refer to an antibody with heavy chains that contain an Fc region. Native antibodies are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, The Pharmacology of Monoclonal Antibodies. Springer Berlin Heidelberg, 1994. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “heavy chain-only antibody” or “HCAb” refers to a functional antibody, which comprises heavy chains, but lacks the light chains usually found in antibodies. Camelid animals (such as camels, llamas, or alpacas) are known to produce HCAbs.

The term “single domain antibody” or “sdAb” refers to an antibody fragment consisting of a single monomeric variable antibody domain. In some cases, single domain antibodies are engineered from camelid HCAbs, and such sdAbs are referred herein as “nanobodies” or “V_{H}Hs”. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual, (Cold Spring Harbor Laboratory Press, 2nd ed. 1988); Hammerling et al., Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381 (1991); Marks et al., J. Mol. Biol. 222:581 (1991). Also available for the preparation of human monoclonal antibodies are methods described in Cole et al., Monoclonal Antibodies and Cancer Therapy, Alan R. Liss, 77 (1985); Boerner et al., J. Immunol. 147(1):86-95 (1991). See also van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA 103:3557-3562 (2006) regarding human antibodies generated via a human B-cell hybridoma technology.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

“Chimeric antigen receptor” or “CAR” as used herein refers to genetically engineered receptors, which graft one or more antigen specificity onto cells, such as T cells. CARs are also known as “artificial T-cell receptors,” “chimeric T cell receptors,” or “chimeric immune receptors.” In some embodiments, the CAR comprises an extracellular variable domain of an antibody specific for a tumor antigen, and an intracellular signaling domain of a T cell or other receptors, such as one or more costimulatory domains. “CAR-T” refers to a T cell that expresses a CAR.

“T cell receptor” or “TCR” as used herein refers to endogenous or recombinant T cell receptor comprising an extracellular antigen binding domain that binds to a specific antigenic peptide bound in an MEW molecule. In some embodiments, the TCR comprises a TCRα polypeptide chain and a TCR β polypeptide chain. In some embodiments, the TCR specifically binds a tumor antigen.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

The term “express” refers to translation of a nucleic acid into a protein. Proteins may be expressed and remain intracellular, become a component of the cell surface membrane, or be secreted into extracellular matrix or medium.

The term “host cell” refers to a cell which can support the replication or expression of the expression vector. Host cells may be prokaryotic cells such as E. coli, or eukaryotic cells, such as yeast, insect cells, amphibian cells, or mammalian cells.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid.

The term “in vivo” refers to inside the body of the organism from which the cell is obtained. “Ex vivo” or “in vitro” means outside the body of the organism from which the cell is obtained.

The term “cell” includes the primary subject cell and its progeny.

“Activation”, as used herein in relation to a cell expressing CD3, refers to the state of the cell that has been sufficiently stimulated to induce a detectable increase in downstream effector functions of the CD3 signaling pathway, including, without limitation, cellular proliferation and cytokine production.

The term “domain” when referring to a portion of a protein is meant to include structurally and/or functionally related portions of one or more polypeptides which make up the protein. For example, a transmembrane domain of a dimeric receptor may refer to the portions of each polypeptide chain of the receptor that span the membrane. A domain may also refer to related portions of a single polypeptide chain. For example, a transmembrane domain of a monomeric receptor may refer to portions of the single polypeptide chain of the receptor that span the membrane. A domain may also include only a single portion of a polypeptide.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “inducible promoter” refers to a promoter whose activity can be regulated by adding or removing one or more specific signals. For example, an inducible promoter may activate transcription of an operably linked nucleic acid under a specific set of conditions, e.g., in the presence of an inducing agent or conditions that activates the promoter and/or relieves repression of the promoter.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of the disease (such as, for example, tumor volume in cancer). The methods of the invention contemplate any one or more of these aspects of treatment.

The term “therapeutically effective amount” refers to an amount of a composition as disclosed herein, effective to “treat” a disease or disorder in an individual. In the case of infectious disease, the therapeutically effective amount of a composition comprising a composition that can improve the patients' condition.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

A “subject” or an “individual” for purposes of treatment refers to any animal classified as a mammal, including humans, domestic and farm animals, and zoo, sports, or pet animals, such as dogs, horses, cats, cows, etc.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “or,” and “the” include plural referents unless the context clearly dictates otherwise.

### The Co-Receptor Systems

The present invention in some embodiments provides an engineered immune cell comprising a chimeric receptor (CR) comprising: i) a CR antigen binding domain specifically recognizing a CR target antigen; ii) a CR transmembrane domain, and iii) an intracellular CR signaling domain, wherein the CR target antigen is selected from the group consisting of CCR5, CXCR4, and CD4. In some embodiments, there is provided an engineered immune cell comprising: a nucleic acid encoding a chimeric receptor (“CR”), wherein the CR comprises: i) a CR antigen binding domain specifically recognizing a CR target antigen; ii) a CR transmembrane domain; and iii) an intracellular CR signaling domain, wherein the CR target antigen is selected from the group consisting of CCR5, CXCR4, and CD4.

Under some circumstances, signals generated through the CR signaling domain alone are insufficient for full activation of the immune cell and that a secondary or co-stimulatory signal is also required. Thus, in some embodiments, immune cell activation is mediated by two distinct classes of intracellular signaling sequence: those that initiate antigen-dependent primary activation through the CR (such as the signaling sequence of the intracellular CR signaling domain) and those that provide a secondary or co-stimulatory signal (referred to herein as “co-stimulatory signaling sequences”). The co-stimulatory signaling sequence can be present in the CR; in other words, the CR can further comprise a CR co-stimulatory domain.

In some embodiments, the co-stimulatory signaling sequence is provided by a co-receptor. Specifically, in some embodiments, there is provided an engineered immune cell comprising: a) a chimeric receptor (CR) comprising: i) a CR antigen binding domain specifically recognizing a CR target antigen; ii) a CR transmembrane domain; and iii) an intracellular CR signaling domain; and b) a chimeric co-receptor (CCOR) comprising: i) a CCOR target antigen binding domain specifically recognizing a CCOR target antigen; ii) a CCR transmembrane domain; and iii) an intracellular CCOR co-stimulatory domain, wherein the CR target antigen is CCR5 or CXCR4 and the CCOR target antigen is CD4, or wherein the CR target antigen is CD4 and the CCOR target antigen is CCR5 or CXCR4. In some embodiments, there is provided an engineered immune cell comprising: a) a first nucleic acid encoding a chimeric receptor (CR), wherein the CR comprises: i) a CR antigen binding domain specifically recognizing a CR target antigen; ii) a CR transmembrane domain; and iii) an intracellular CR signaling domain; and b) a second nucleic acid encoding a chimeric co-receptor (CCOR), wherein the CCOR comprises: i) a CCOR target antigen binding domain specifically recognizing a CCOR target antigen; ii) a CCR transmembrane domain; and iii) an intracellular CCOR co-stimulatory domain, wherein the CR target antigen is CCR5 or CXCR4 and the CCOR target antigen is CD4, or wherein the CR target antigen is CD4 and the CCOR target antigen is CCR5 or CXCR4. In some embodiments, the CR and CCOR are expressed from the nucleic acid and localized to the immune cell surface. In some embodiments, the immune cell is a T cell. In some embodiments, the CR does not comprise a co-stimulatory domain. In some embodiments, the immune cell is modified to block or decrease the expression of CCR5 of the immune cell. In some embodiments, the immune cell is modified to block or decrease the expression of one or both of the endogenous TCR subunits of the immune cell. Modifications of cells to disrupt gene expression include any such techniques known in the art, including for example RNA interference (e.g., siRNA, shRNA, miRNA), gene editing (e.g., CRISPR- or TALEN-based gene knockout), and the like.

In some embodiments, the immune cells are modified to further comprise one or more co-receptors. Thus, for example, in some embodiments, there is provided an engineered immune cell comprising: a) a chimeric receptor (CR) comprising: i) a CR antigen binding domain specifically recognizing a CR target antigen; ii) a CR transmembrane domain; and iii) an intracellular CR signaling domain, wherein the CR target antigen is selected form the group consisting of CCR5, CXCR4, and CD4; and b) a co-receptor (COR) selected from the group consisting of CXCR5, α4β7, CCR9, or a combination thereof. In some embodiments, there is provided an engineered immune cell comprising: a) a chimeric receptor (CR) comprising: i) a CR antigen binding domain specifically recognizing a CR target antigen; ii) a CR transmembrane domain; iii) an intracellular CR co-stimulatory domain; and iv) an intracellular CR signaling domain, wherein the CR target antigen is selected form the group consisting of CCR5, CXCR4, and CD4; and b) a co-receptor (COR) selected from the group consisting of CXCR5, α4β7, CCR9, or a combination thereof. In some embodiments, the immune cell comprises both α4β7 and CCR9. In some embodiments, the immune cell comprises all of CXCR5, α4β7, and CCR9. In some embodiments, the immune cell is a T cell. In some embodiments, the immune cell is modified to block or decrease the expression of CCR5 of the immune cell. In some embodiments, the immune cell is modified to block or decrease the expression of one or both of the endogenous TCR subunits of the immune cell.

In some embodiments, there is provided an engineered immune cell comprising: a) a nucleic acid encoding a chimeric receptor (CR), wherein the CR comprises: i) a CR antigen binding domain specifically recognizing a CR target antigen; ii) a CR transmembrane domain; and iii) an intracellular CR signaling domain, wherein the CR target antigen is selected from the group consisting of CCR5, CXCR4, and CD4; and b) a nucleic acid encoding a co-receptor (COR), wherein the COR is selected from the group consisting of CXCR5, α4β7, CCR9, or a combination thereof. In some embodiments, there is provided an engineered immune cell comprising: a) a nucleic acid encoding a chimeric receptor (CR), wherein the CR comprises: i) a CR antigen binding domain specifically recognizing a CR target antigen; ii) a CR transmembrane domain; iii) an intracellular CR co-stimulatory domain; and iv) an intracellular CR signaling domain, wherein the CR target antigen is selected from the group consisting of CCR5, CXCR4, and CD4; and b) a nucleic acid encoding a co-receptor (COR), wherein the COR is selected from the group consisting of CXCR5, α4β7, CCR9, or a combination thereof. In some embodiments, the immune cell comprises a nucleic acid encoding α4β7 and a nucleic acid encoding CCR9. In some embodiments, the immune cell comprises a nucleic acid encoding CXCR5, a nucleic acid encoding α4β7, and a nucleic acid encoding CCR9. In some embodiments, the CR and COR are expressed from the nucleic acid and localized to the immune cell surface. In some embodiments, the immune cell is a T cell. In some embodiments, the immune cell is modified to block or decrease the expression of CCR5 of the immune cell. In some embodiments, the immune cell is modified to block or decrease the expression of one or both of the endogenous TCR subunits of the immune cell.

In some embodiments, there is provided an engineered immune cell comprising: a) a nucleic acid encoding a chimeric receptor (CR), wherein the CR comprises: i) a CR antigen binding domain comprising a CD4 binding moiety (such as an anti-CD4 antibody moiety, for example a scFv or sdAb) and a CCR5 binding moiety (such as an anti-CCR5 antibody moiety, for example a scFv or sdAb); ii) a CR transmembrane domain; and iii) an intracellular CR signaling domain; and b) a nucleic acid encoding a co-receptor (COR), wherein the COR is selected from the group consisting of CXCR5, α4β7, CCR9, or a combination thereof. In some embodiments, there is provided an engineered immune cell comprising: a) a nucleic acid encoding a chimeric receptor (CR), wherein the CR comprises: i) a CR antigen binding domain comprising a CD4 binding moiety (such as an anti-CD4 antibody moiety, for example a scFv or sdAb) and a CCR5 binding moiety (such as an anti-CCR5 antibody moiety, for example a scFv or sdAb); ii) a CR transmembrane domain; iii) an intracellular CR co-stimulatory domain; and iv) an intracellular CR signaling domain, wherein the CR target antigen is selected from the group consisting of CCR5, CXCR4, and CD4; and b) a nucleic acid encoding a co-receptor (COR), wherein the COR is selected from the group consisting of CXCR5, α4β7, CCR9, or a combination thereof. In some embodiments, the CD4 binding moiety and the CCR5 binding moiety are linked in tandem. In some embodiments, the CD4 binding moiety is N-terminal to the anti-CCR5 moiety. In some embodiments, the CD4 binding moiety is C-terminal to the anti-CCR5 moiety. In some embodiments, the immune cell comprises a nucleic acid encoding α4β7 and a nucleic acid encoding CCR9. In some embodiments, the immune cell comprises a nucleic acid encoding CXCR5, a nucleic acid encoding α4β7, and a nucleic acid encoding CCR9. In some embodiments, the CR and COR are expressed from the nucleic acid and localized to the immune cell surface. In some embodiments, the immune cell is a T cell. In some embodiments, the immune cell is modified to block or decrease the expression of CCR5 of the immune cell. In some embodiments, the immune cell is modified to block or decrease the expression of one or both of the endogenous TCR subunits of the immune cell.

In some embodiments, there is provided an engineered immune cell comprising: a) a nucleic acid encoding a chimeric receptor (CR), wherein the CR comprises: i) a CR antigen binding domain comprising a CD4 binding moiety (such as an anti-CD4 antibody moiety, for example a scFv or sdAb) and a broadly neutralizing antibody (“bNAb”) moiety (such as scFv or sdAb); ii) a CR transmembrane domain; and iii) an intracellular CR signaling domain; and b) a nucleic acid encoding a co-receptor (COR), wherein the COR is selected from the group consisting of CXCR5, α4β7, CCR9, or a combination thereof. In some embodiments, there is provided an engineered immune cell comprising: a) a nucleic acid encoding a chimeric receptor (CR), wherein the CR comprises: i) a CR antigen binding domain comprising a CD4 binding moiety (such as an anti-CD4 antibody moiety, for example a scFv or sdAb) and a broadly neutralizing antibody (“bNAb”) moiety (such as scFv or sdAb); ii) a CR transmembrane domain; iii) an intracellular CR co-stimulatory domain; and iv) an intracellular CR signaling domain, and b) a nucleic acid encoding a co-receptor (COR), wherein the COR is selected from the group consisting of CXCR5, α4β7, CCR9, or a combination thereof. In some embodiments, the CD4 binding moiety and the bNAb moiety are linked in tandem. In some embodiments, the CD4 binding moiety is N-terminal to the bNAb moiety. In some embodiments, the CD4 binding moiety is C-terminal to the bNAb moiety. In some embodiments, the immune cell comprises a nucleic acid encoding α4β7 and a nucleic acid encoding CCR9. In some embodiments, the immune cell comprises a nucleic acid encoding CXCR5, a nucleic acid encoding α4β7, and a nucleic acid encoding CCR9. In some embodiments, the CR and COR are expressed from the nucleic acid and localized to the immune cell surface. In some embodiments, the immune cell is a T cell. In some embodiments, the immune cell is modified to block or decrease the expression of CCR5 of the immune cell. In some embodiments, the immune cell is modified to block or decrease the expression of one or both of the endogenous TCR subunits of the immune cell.

In some embodiments, there is provided an engineered immune cell comprising: a) a nucleic acid encoding a chimeric receptor (CR), wherein the CR comprises: i) a CR antigen binding domain comprising a CCR5 binding moiety (such as an anti-CCR5 antibody moiety, for example an scFv or sdAb) and a broadly neutralizing antibody (“bNAb”) moiety (such as scFv or sdAb); ii) a CR transmembrane domain; and iii) an intracellular CR signaling domain; and b) a nucleic acid encoding a co-receptor (COR), wherein the COR is selected from the group consisting of CXCR5, α4β7, CCR9, or a combination thereof. In some embodiments, there is provided an engineered immune cell comprising: a) a nucleic acid encoding a chimeric receptor (CR), wherein the CR comprises: i) a CR antigen binding domain comprising a CCR5 binding moiety (such as an anti-CCCR5 antibody moiety, for example a scFv or sdAb) and a broadly neutralizing antibody (“bNAb”) moiety (such as scFv or sdAb); ii) a CR transmembrane domain; iii) an intracellular CR co-stimulatory domain; and iv) an intracellular CR signaling domain, and b) a nucleic acid encoding a co-receptor (COR), wherein the COR is selected from the group consisting of CXCR5, α4β7, CCR9, or a combination thereof. In some embodiments, the CCR5 binding moiety and the bNAb moiety are linked in tandem. In some embodiments, the CCR5 binding moiety is N-terminal to the bNAb moiety. In some embodiments, the CCR5 binding moiety is C-terminal to the bNAb moiety. In some embodiments, the immune cell comprises a nucleic acid encoding α4β7 and a nucleic acid encoding CCR9. In some embodiments, the immune cell comprises a nucleic acid encoding CXCR5, a nucleic acid encoding α4β7, and a nucleic acid encoding CCR9. In some embodiments, the CR and COR are expressed from the nucleic acid and localized to the immune cell surface. In some embodiments, the immune cell is a T cell. In some embodiments, the immune cell is modified to block or decrease the expression of CCR5 of the immune cell. In some embodiments, the immune cell is modified to block or decrease the expression of one or both of the endogenous TCR subunits of the immune cell.

In some embodiments, the immune cell is engineered to express CR, CCOR, and one or more CORs described herein. Thus, for example, in some embodiments, there is provided an engineered immune cell comprising: a) a chimeric receptor (CR) comprising: i) a CR antigen binding domain specifically recognizing a CR target antigen; ii) a CR transmembrane domain; and iii) an intracellular CR signaling domain; b) a chimeric co-receptor (CCOR) comprising: i) a CCOR target antigen binding domain specifically recognizing a CCOR target antigen; ii) a CCR transmembrane domain; and iii) an intracellular CCOR co-stimulatory domain, and c) a co-receptor (COR), wherein the CR target antigen is CCR5 or CXCR4 and the CCOR target antigen is CD4, or wherein the CR target antigen is CD4 and the CCOR target antigen is CCR5 or CXCR4. In some embodiments, the COR is selected from the group consisting of CXCR5, α4β7, CCR9, or a combination thereof. In some embodiments, the immune cell comprises both α4β7 and CCR9. In some embodiments, the immune cell comprises all of CXCR5, α4β7, and CCR9. In some embodiments, the CR does not comprise a CR intracellular signaling domain. In some embodiments, the immune cell is a T cell. In some embodiments, the immune cell is modified to block or decrease the expression of CCR5 of the immune cell. In some embodiments, the immune cell is modified to block or decrease the expression of one or both of the endogenous TCR subunits of the immune cell.

In some embodiments, there is provided an engineered immune cell comprising: a) a first nucleic acid encoding a chimeric receptor (CR), wherein the CR comprises: i) a CR antigen binding domain specifically recognizing a CR target antigen; ii) a CR transmembrane domain; and iii) an intracellular CR signaling domain; and b) a second nucleic acid encoding a chimeric co-receptor (CCOR), wherein the CCOR comprises: i) a CCOR target antigen binding domain specifically recognizing a CCOR target antigen; ii) a CCR transmembrane domain; and iii) an intracellular CCOR co-stimulatory domain, and c) a third nucleic acid encoding a co-receptor (COR); wherein the CR target antigen is CCR5 or CXCR4 and the CCOR target antigen is CD4, or wherein the CR target antigen is CD4 and the CCOR target antigen is CCR5 or CXCR4. In some embodiments, the COR is selected from the group consisting of CXCR5, α4β7, CCR9, or a combination thereof. In some embodiments, the immune cell comprises a nucleic acid encoding α4β7 and a nucleic acid encoding CCR9. In some embodiments, the immune cell comprises a nucleic acid encoding CXCR5, a nucleic acid encoding α4β7, and a nucleic acid encoding CCR9. In some embodiments, the CR, CCOR, and COR are expressed from the nucleic acid and localized to the immune cell surface. In some embodiments, the CR does not comprise a CR intracellular signaling domain. In some embodiments, the immune cell is a T cell. In some embodiments, the immune cell is modified to block or decrease the expression of CCR5 of the immune cell. In some embodiments, the immune cell is modified to block or decrease the expression of one or both of the endogenous TCR subunits of the immune cell.

In some embodiments, there are provided nucleic acids comprising a nucleic acid sequence encoding a CR, a nucleic acid sequence encoding a CCOR, and/or a nucleic acid sequence encoding a COR. In some embodiments, the CR, CCOR, and COR nucleic acid sequences are each contained in different vectors. In some embodiments, some or all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses). In some embodiments, one or more of the vectors is integrated into the host genome of the immune cell. In some embodiments, the CR, CCOR, and/or COR nucleic acid sequences are each under the control of different promoters. In some embodiments, the promoters have the same sequences. In some embodiments, the promoters have different sequences. In some embodiments, some or all of the nucleic acid sequences are under the control of a single promoter. In some embodiments, some or all of the promoters are inducible. For example, the CCOR and COR nucleic acids can be under the control of a promoter that is inducible upon activation of the immune cell. In some embodiments, some or all of the promoters are constitutive.

In some embodiments according to any of the above embodiments, the engineered immune cell is selected from the group consisting of T cells, B cells, NK cells, dendritic cells, eosinophils, macrophages, lymphoid cells, and mast cells. In some embodiments, the engineered immune cell is selected from a cytotoxic T cell, a helper T cell, and a natural killer T cell. In some embodiments, the engineered immune cell is a cytotoxic T cell. In some embodiments, the engineered immune cells are enriched for CD4 expression. In some embodiments, the engineered immune cells are enriched for CD8 expression.

In some embodiments, the engineered immune cells are derived from primary immune cells. In some embodiments, the engineered immune cells are derived from cells (e.g., iPS cells) that are artificially induced to possess immune activities. In some embodiments, the engineered immune cells are derived from CD4+ immune cells (or immune cells enriched for CD4 expression). In some embodiments, the engineered immune cells are derived from CD8+ immune cells (or immune cells enriched for CD8 expression).

In some embodiments when two or more of the nucleic acids encoding CR, CCOR, and COR are under the control of a single promoter, the nucleic acids can be connected via a linker selected from the group consisting of an internal ribosomal entry site (IRES) and a nucleic acid encoding a self-cleaving 2A peptide (such as P2A, T2A, E2A, or F2A).

### Chimeric Receptor (CR) Constructs

The CR described herein comprises a CR antigen binding domain that specifically recognizes a CR target antigen, a CR transmembrane domain, and an intracellular CR signaling domain.

In some embodiments, the CR antigen binding domain is fused to the CR transmembrane domain directly or indirectly. For example, the CR can be a single polypeptide that comprises, from N- to C-termini: the CR antigen binding domain, the CR transmembrane domain, and the CR intracellular signaling domain. The CR antigen binding domain, CR transmembrane domain, and CR intracellular domain can be fused directly to each other or indirectly via linker sequences.

In some embodiments, the CR antigen binding domain is non-covalently bound to a polypeptide comprising the CR transmembrane domain. This can be accomplished, for example, by using two members of a binding pair, one fused to the CR antigen binding domain (e.g., fused to the C-terminus of the CR antibody binding domain), the other fused to the CR transmembrane domain (e.g., fused to the N-terminus of the CR transmembrane domain). The two components are brought together through interaction of the two members of the binding pair. For example, the CR can comprise an extracellular domain comprising: i) a first polypeptide comprising the CR antigen binding domain and a first member of a binding pair; and ii) a second polypeptide comprising a second member of the binding pair, wherein the first member and the second member bind to each other non-covalently. The first member of the binding pair can be fused to the CR antigen binding domain directly or indirectly. Similarly, the second member of the binding pair can be fused to the CR transmembrane domain directly or indirectly. Suitable binding pairs include, but are not limited to, leucine zipper, biotin/streptavidin, MIC ligand/iNKG2D etc. See Cell 173, 1426-1438, Oncoimmunology. 2018; 7(1): e1368604, U.S. Pat. No. 10,259,858B2.

In some embodiments, the CR antigen binding domain comprises two or more antigen binding domains. For example, in some embodiments, the CR antigen binding domain comprises a CD4 binding moiety and a CCR5 binding moiety linked in tandem. In some embodiments, the CR antigen binding domain comprises a CD4 binding moiety and a bNAb moiety linked in tandem. In some embodiments, the CR antigen binding domain comprises a CCR5 binding moiety and a bNAb moiety linked in tandem. In some embodiments, the CD4 binding moiety, CCR5 binding moiety, and/or bNAb moiety is selected from the group consisting of scFv or sdAb.

The intracellular CR signaling domain in some embodiments comprises a functional primary immune cell signaling sequences, which include, but are not limited to, those found in a protein selected from the group consisting of CD3ζ, FcRγ, FcRβ, CD3γ, CD3δ, CD3ε, CD5, CD22, CD79a, CD79b, and CD66d. A “functional” primary immune cell signaling sequence is a sequence that is capable of transducing an immune cell activation signal when operably coupled to an appropriate receptor. “Non-functional” primary immune cell signaling sequences, which may comprises fragments or variants of primary immune cell signaling sequences, are unable to transduce an immune cell activation signal. The CCORs described herein lack a functional primary immune cell signaling sequence. In some embodiments, the CCORs lack any primary immune cell signaling sequence.

In some embodiments, the CR transmembrane domain comprises one or more transmembrane domains derived from, for example, CD28, CD3ε, CD3ζ, CD45, CD4, CD5, CD8, CD9, CD16, CD22, CD33, CD37, CD64, CD80, CD86, CD134, CD137, or CD154.

The CR antigen binding domain can be an antibody moiety or a ligand that specifically recognizing the CR target antigen. In some embodiments, the CR antigen binding domain specifically binds to a target antigen with a) an affinity that is at least about 10 (including for example at least about any of 10, 20, 30, 40, 50, 75, 100, 200, 300, 400, 500, 750, 1000 or more) times its binding affinity for other molecules; orb) a K_{d} no more than about 1/10 (such as no more than about any of 1/10, 1/20, 1/30, 1/40, 1/50, 1/75, 1/100, 1/200, 1/300, 1/400, 1/500, 1/750, 1/1000 or less) times its K_{d} for binding to other molecules. Binding affinity can be determined by methods known in the art, such as ELISA, fluorescence activated cell sorting (FACS) analysis, or radioimmunoprecipitation assay (RIA). K_{d} can be determined by methods known in the art, such as surface plasmon resonance (SPR) assay utilizing, for example, Biacore instruments, or kinetic exclusion assay (KinExA) utilizing, for example, Sapidyne instruments.

In some embodiments, the CR antigen binding domain is selected from the group consisting of Fab, a Fab′, a (Fab′)₂, an Fv, a single chain Fv (scFv), a single domain antibody (sdAb), and a peptide ligand specifically binding to the CR target antigen.

In some embodiments, the CR antigen binding domain is an antibody moiety. In some embodiments, the antibody moiety is monospecific. In some embodiments, the antibody moiety is multi-specific. In some embodiments, the antibody moiety is bispecific. In some embodiments, the antibody moiety is a tandem scFv, a diabody (Db), a single chain diabody (scDb), a dual-affinity retargeting (DART) antibody, a dual variable domain (DVD) antibody, a chemically cross-linked antibody, a heteromultimeric antibody, or a heteroconjugate antibody. In some embodiments, the antibody moiety is a scFv. In some embodiments, the antibody moiety is a single domain antibody (sdAb). In some embodiments, the antibody moiety is fully human, semi-synthetic with human antibody framework regions, or humanized.

The antibody moiety in some embodiments comprises specific CDR sequences derived from one or more antibody moieties (such as a monoclonal antibody) or certain variants of such sequences comprising one or more amino acid substitutions. In some embodiments, the amino acid substitutions in the variant sequences do not substantially reduce the ability of the antigen-binding domain to bind the target antigen. Alterations that substantially improve target antigen binding affinity or affect some other property, such as specificity and/or cross-reactivity with related variants of the target antigen, are also contemplated.

In some embodiments, the CR antigen binding domain binds the CR target antigen with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values).

In some embodiments, for example, when expressed alone or co-expressed with a COR without a CCOR, the CR may further comprise an intracellular CR co-stimulatory domain. The intracellular CR co-stimulatory domain can be a portion of the intracellular domain of a co-stimulatory molecule including, for example, CD28, 4-1BB (CD137), CD27, OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, and a ligand that specifically binds with CD83. In some embodiments, the intracellular CR co-stimulatory domain comprises a fragment of 4-1BB. In some embodiments, the intracellular CCOR co-stimulatory domain comprises a fragment of CD28 and a fragment of 4-1BB. In some embodiments, for example when co-expressed with a CCOR, the CR does not comprise a functional co-stimulatory domain.

Thus, for example, in some embodiments, there is provided a chimeric receptor (CR) comprising: i) a CR antigen binding domain specifically recognizing CD4; ii) a CR transmembrane domain, and iii) an intracellular CR signaling domain. In some embodiments, there is provided an engineered immune cell comprising: a chimeric receptor (CR) comprising: i) a CR antigen binding domain specifically recognizing CD4; ii) a CR transmembrane domain, and iii) an intracellular CR signaling domain. In some embodiments, there is provided an engineered immune cell comprising: a nucleic acid encoding a chimeric receptor (CR), wherein the CR comprises: i) a CR antigen binding domain specifically recognizing CD4; ii) a CR transmembrane domain, and iii) an intracellular CR signaling domain. In some embodiments, the engineered immune cell further comprises one or more COR (such as CXCR5) or a nucleic acid encoding one or more COR (such as CXCR5). In some embodiments, the engineered immune cell further comprises a broadly neutralizing antibody (bNAb) or a nucleic acid encoding a bNAb.

In some embodiments, there is provided a chimeric receptor (CR) comprising: i) a CR antigen binding domain specifically recognizing CCR5; ii) a CR transmembrane domain, and iii) an intracellular CR signaling domain. In some embodiments, there is provided an engineered immune cell comprising: a chimeric receptor (CR) comprising: i) a CR antigen binding domain specifically recognizing CCR5; ii) a CR transmembrane domain, and iii) an intracellular CR signaling domain. In some embodiments, there is provided an engineered immune cell comprising: a nucleic acid encoding a chimeric receptor (CR), wherein the CR comprises: i) a CR antigen binding domain specifically recognizing CCR5; ii) a CR transmembrane domain, and iii) an intracellular CR signaling domain. In some embodiments, the engineered immune cell further comprises one or more COR (such as CXCR5) or a nucleic acid encoding one or more COR (such as CXCR5). In some embodiments, the engineered immune cell further comprises a broadly neutralizing antibody (bNAb) or a nucleic acid encoding a bNAb.

In some embodiments, there is provided a chimeric receptor (CR) comprising: i) a CR antigen binding domain comprising a CD4 binding moiety (such as an anti-CD4 antibody moiety, for example a scFv or sdAb) and a CCR5 binding moiety (such as an anti-CCR5 antibody moiety, for example a scFv or sdAb); ii) a CR transmembrane domain, and iii) an intracellular CR signaling domain. In some embodiments, there is provided an engineered immune cell comprising: a chimeric receptor (CR) comprising: i) a CR antigen binding domain comprising a CD4 binding moiety (such as an anti-CD4 antibody moiety, for example a scFv or sdAb) and a CCR5 binding moiety (such as an anti-CCR5 antibody moiety, for example a scFv or sdAb); ii) a CR transmembrane domain, and iii) an intracellular CR signaling domain. In some embodiments, there is provided an engineered immune cell comprising: a nucleic acid encoding a chimeric receptor (CR), wherein the CR comprises: i) a CR antigen binding domain comprising a CD4 binding moiety (such as an anti-CD4 antibody moiety, for example a scFv or sdAb) and a CCR5 binding moiety (such as an anti-CCR5 antibody moiety, for example a scFv or sdAb); ii) a CR transmembrane domain, and iii) an intracellular CR signaling domain. In some embodiments, the CD4 binding moiety and the CCR5 binding moiety are linked in tandem. In some embodiments, the CD4 binding moiety is N-terminal to the CCR5 moiety. In some embodiments, the CD4 binding moiety is C-terminal to the CCR5 moiety. In some embodiments, the engineered immune cell further comprises one or more COR (such as CXCR5) or a nucleic acid encoding one or more COR (such as CXCR5). In some embodiments, the engineered immune cell further comprises a broadly neutralizing antibody (bNAb) or a nucleic acid encoding a bNAb.

In some embodiments, the CR described herein is a chimeric antigen receptor (“CAR”). Thus, for example, in some embodiments, there is provided an anti-CD4 CAR comprising: i) a CR antigen binding domain specifically recognizing CD4 (for example an anti-CD4 antibody moiety such as scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising an anti-CD4 CAR comprising: i) a CR antigen binding domain specifically recognizing CD4 (for example an anti-CD4 antibody moiety such as scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising: a nucleic acid encoding an anti-CD4 CAR comprising: i) a CR antigen binding domain specifically recognizing CD4 (for example an anti-CD4 antibody moiety such as scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, the CR antigen domain specifically recognizes domain 1. For example, the CR antigen domain can be an anti-CD4 antibody (e.g., scFv or sdAb) specifically recognizing domain 1 of CD4. In some embodiments, the engineered immune cell further comprises a broadly neutralizing antibody (bNAb) or a nucleic acid encoding a bNAb.

In some embodiments, there is provided an anti-CCR5 CAR comprising: i) a CR antigen binding domain specifically recognizing CCR5 (for example an anti-CCR5 antibody moiety such as scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising an anti-CCR5 CAR comprising: i) a CR antigen binding domain specifically recognizing CCR5 (for example an anti-CCR5 antibody moiety such as scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising: a nucleic acid encoding an anti-CCR5 CAR comprising: i) a CR antigen binding domain specifically recognizing CCR5 (for example an anti-CCR5 antibody moiety such as scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, the engineered immune cell further comprises a broadly neutralizing antibody (bNAb) or a nucleic acid encoding a bNAb.

In some embodiments, there is provided a tandem anti-CD4 anti-CCR5 CAR comprising: i) a CR antigen binding domain comprising a CD4 binding moiety (such as an anti-CD4 antibody moiety, for example an scFv or sdAb) and a CCR5 binding moiety (such as an anti-CCR5 antibody moiety, for example an scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising a tandem anti-CD4 anti-CCR5 CAR comprising: i) a CR antigen binding domain comprising a CD4 binding moiety (such as an anti-CD4 antibody moiety, for example an scFv or sdAb) and a CCR5 binding moiety (such as an anti-CCR5 antibody moiety, for example an scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising: a nucleic acid encoding a tandem anti-CD4 anti-CCR5 CAR comprising: i) a CR antigen binding domain comprising a CD4 binding moiety (such as an anti-CD4 antibody moiety, for example an scFv or sdAb) and a CCR5 binding moiety (such as an anti-CCR5 antibody moiety, for example an scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, the CD4 binding moiety and the CCR5 binding moiety are linked in tandem. In some embodiments, the CD4 binding moiety is N-terminal to the CCR5 binding-moiety. In some embodiments, the CD4 binding moiety is C-terminal to the CCR5 binding moiety. In some embodiments, the CR antigen domain specifically recognizes domain 1. For example, the CR antigen domain can comprise an anti-CD4 antibody (e.g., scFv or sdAb) specifically recognizing domain 1 of CD4. In some embodiments, the engineered immune cell further comprises a broadly neutralizing antibody (bNAb) or a nucleic acid encoding a bNAb.

In some embodiments, there is provided a tandem anti-CD4 bNAb CAR comprising: i) a CR antigen binding domain comprising a CD4 binding moiety (such as an anti-CD4 antibody moiety, for example a scFv or sdAb) and a bNAb moiety (such as a scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising an anti-CD4 bNAb CAR comprising: i) a CR antigen binding domain comprising a CD4 binding moiety (such as an anti-CD4 antibody moiety, for example a scFv or sdAb) and a bNAb moiety (such as a scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising: a nucleic acid encoding an anti-CD4 bNAb CAR comprising: i) a CR antigen binding domain comprising a CD4 binding moiety (such as an anti-CD4 antibody moiety, for example a scFv or sdAb) and a bNAb moiety (such as a scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, the CD4 binding moiety and the bNAb moiety are linked in tandem. In some embodiments, the CD4 binding moiety is N-terminal to the bNAb moiety. In some embodiments, the CD4 binding moiety is C-terminal to the bNAb moiety. In some embodiments, the CR antigen domain specifically recognizes domain 1. For example, the CR antigen domain can comprise an anti-CD4 antibody (e g , scFv or sdAb) specifically recognizing domain 1 of CD4. In some embodiments, the engineered immune cell further comprises a broadly neutralizing antibody (bNAb) or a nucleic acid encoding a bNAb, including VRC01, PGT121, 3BNC117, 10-1074, N6, VRC07, VRC07-523, eCD4-IG, 10E8, 10E8v4, PG9, PGDM 1400, PGT151, CAP256.25, 35O22, 8ANC195, and the like.

In some embodiments, there is provided a tandem anti-CCR5 bNAb CAR comprising: i) a CR antigen binding domain comprising a CCR5 binding moiety (such as an anti-CCR5 antibody moiety, for example a scFv or sdAb) and a bNAb moiety (such as a scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising an anti-CCR5 bNAb CAR comprising: i) a CR antigen binding domain comprising a CCR5 binding moiety (such as an anti-CCR5 antibody moiety, for example a scFv or sdAb) and a bNAb moiety (such as a scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, there is provided an engineered immune cell comprising: a nucleic acid encoding an anti-CCR5 bNAb CAR comprising: i) a CR antigen binding domain comprising a CCR5 binding moiety (such as an anti-CCR5 antibody moiety, for example a scFv or sdAb) and a bNAb moiety (such as a scFv or sdAb); ii) an optional hinge sequence (such as a hinge sequence derived from CD8); iii) a CR transmembrane domain (such as a CD8 transmembrane domain), iv) an intracellular co-stimulatory domain (such as a co-stimulatory domain derived from 4-1BB or CD28); and v) an intracellular CR signaling domain (such as an intracellular signaling domain derived from CD3ζ). In some embodiments, the CCR5 binding moiety and the bNAb moiety are linked in tandem. In some embodiments, the CCR5 binding moiety is N-terminal to the bNAb moiety. In some embodiments, the CCR5 binding moiety is C-terminal to the bNAb moiety. In some embodiments, the engineered immune cell further comprises a broadly neutralizing antibody (bNAb) or a nucleic acid encoding a bNAb, including VRC01, PGT121, 3BNC117, 10-1074, N6, VRC07, VRC07-523, eCD4-IG, 10E8, 10E8v4, PG9, PGDM 1400, PGT151, CAP256.25, 35O22, 8ANC195, and the like.

In some embodiments, the CR described herein is a chimeric TCR receptor (“cTCR”). cTCRs typically comprise a CR antigen binding domain fused (directly or indirectly) to the full length or a portion of a TCR subunit, such as TCRα, TCRβ, TCRγ, TCRδ, CD3γ, CD3ε, and CD3δ. The fusion polypeptide can be incorporated into a functional TCR complex along with other TCR subunits and confers antigen specificity to the TCR complex. In some embodiments, the CR antigen binding domain is fused to the full length or a portion of the CD3ε subunit (referred to as “eTCR”). The intracellular CR signaling domain of the cTCR can be derived from the intracellular signaling domain of a TCR subunit. The CR transmembrane domain derived from a TCR subunit. In some embodiments, the intracellular CR signaling domain and the CR transmembrane domain are derived from the same TCR subunit. In some embodiments, the intracellular CR signaling domain and the CR transmembrane domain are derived from CD3ε. In some embodiments, the CR antigen binding domain and the TCR subunit (or a portion thereof) can be fused via a linker (such as a GS linker). In some embodiments, the cTCR further comprises an extracellular domain of a TCR subunit or a portion thereof, which can be the same or different from the TCR unit from which the intracellular CR signaling domain and/or CR transmembrane domain are derived from.

Thus, for example, in some embodiments, there is provided an anti-CD4 cTCR comprising: i) a CR antigen binding domain specifically recognizing CD4 (for example an anti-CD4 antibody moiety such as scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising an anti-CD4 cTCR comprising: i) a CR antigen binding domain specifically recognizing CD4 (for example an anti-CD4 antibody moiety such as scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising: a nucleic acid encoding anti-CD4 cTCR comprising: i) a CR antigen binding domain specifically recognizing CD4 (for example an anti-CD4 antibody moiety such as scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, the CR antigen domain specifically recognizes domain 1. For example, the CR antigen domain can be an anti-CD4 antibody (e.g., scFv or sdAb) specifically recognizing domain 1 of CD4. In some embodiments, the TCR subunit is selected from the group consisting of TCRα, TCRβ, TCRγ, TCRδ, CD3γ, CD3ε. In some embodiments, the CR transmembrane domain, the intracellular CR signaling domain, and the optional extracellular domain of a TCR subunit or a portion thereof are derived from the same TCR subunit. In some embodiments, the CR transmembrane domain, the intracellular CR signaling domain, and the optional extracellular domain of a TCR subunit or a portion thereof are derived from CD3ε. In some embodiments, the cTCR comprises the CR antigen binding domain fused to the N-terminus of a full length CD3ε. In some embodiments, the engineered immune cell further comprises a broadly neutralizing antibody (bNAb) or a nucleic acid encoding a bNAb.

In some embodiments, there is provided an anti-CCR5 cTCR comprising: i) a CR antigen binding domain specifically recognizing CCR5 (for example an anti-CCR5 antibody moiety such as scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising an anti-CCR5 cTCR comprising: i) a CR antigen binding domain specifically recognizing CCR5 (for example an anti-CCR5 antibody moiety such as scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising: a nucleic acid encoding anti-CCR5 cTCR comprising: i) a CR antigen binding domain specifically recognizing CCR5 (for example an anti-CCR5 antibody moiety such as scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, the TCR subunit is selected from the group consisting of TCRα, TCRβ, TCRγ, TCRδ, CD3γ, CD3ε. In some embodiments, the CR transmembrane domain, the intracellular CR signaling domain, and the optional extracellular domain of a TCR subunit or a portion thereof are derived from the same TCR subunit. In some embodiments, the CR transmembrane domain, the intracellular CR signaling domain, and the optional extracellular domain of a TCR subunit or a portion thereof are derived from CD3ε. In some embodiments, the cTCR comprises the CR antigen binding domain fused to the N-terminus of a full length CD3ε. In some embodiments, the engineered immune cell further comprises a broadly neutralizing antibody (bNAb) or a nucleic acid encoding a bNAb.

In some embodiments, there is provided a tandem anti-CD4 anti-CCR5 cTCR comprising: i) a CR antigen binding domain comprising a CD4 binding moiety (such as an anti-CD4 antibody moiety, for example a scFv or sdAb) and a CCR5 binding moiety (such as an anti-CCR5 antibody moiety, for example a scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising a tandem anti-CD4 anti-CCR5 cTCR comprising: i) a CR antigen binding domain comprising a CD4 binding moiety (such as an anti-CD4 antibody moiety, for example a scFv or sdAb) and a CCR5 binding moiety (such as an anti-CCR5 antibody moiety, for example a scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising: a nucleic acid encoding a tandem anti-CD4 anti-CCR5 cTCR comprising: i) a CR antigen binding domain comprising a CD4 binding moiety (such as an anti-CD4 antibody moiety, for example a scFv or sdAb) and a CCR5 binding moiety (such as an anti-CCR5 antibody moiety, for example a scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit; ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, the CR antigen domain specifically recognizes domain 1. For example, the CR antigen domain can be an anti-CD4 antibody (e.g., scFv or sdAb) specifically recognizing domain 1 of CD4. In some embodiments, the TCR subunit is selected from the group consisting of TCRα, TCRβ, TCRγ, TCRδ, CD3γ, CD3ε. In some embodiments, the CR transmembrane domain, the intracellular CR signaling domain, and the optional extracellular domain of a TCR subunit or a portion thereof are derived from the same TCR subunit. In some embodiments, the CR transmembrane domain, the intracellular CR signaling domain, and the optional extracellular domain of a TCR subunit or a portion thereof are derived from CD3ε. In some embodiments, the cTCR comprises the CR antigen binding domain fused to the N-terminus of a full length CD3ε. In some embodiments, the CD4 binding moiety and the CCR5 binding moiety are linked in tandem. In some embodiments, the CD4 binding moiety is N-terminal to the CCR5 moiety. In some embodiments, the CD4 binding moiety is C-terminal to the CCR5 moiety. In some embodiments, the engineered immune cell further comprises a broadly neutralizing antibody (bNAb) or a nucleic acid encoding a bNAb.

In some embodiments, there is provided a tandem anti-CD4 bNAb cTCR comprising: i) a CR antigen binding domain comprising a CD4 binding moiety (such as an anti-CD4 antibody moiety, for example a scFv or sdAb) and a bNAb moiety (such as a scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising a tandem anti-CD4 bNAb cTCR comprising: i) a CR antigen binding domain comprising a CD4 binding moiety (such as an anti-CD4 antibody moiety, for example a scFv or sdAb) and a bNAb moiety (such as a scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising: a nucleic acid encoding a tandem anti-CD4 bNAb cTCR comprising: i) a CR antigen binding domain comprising a CD4 binding moiety (such as an anti-CD4 antibody moiety, for example a scFv or sdAb) and a bNAb moiety (such as a scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit; ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, the CR antigen domain specifically recognizes domain 1. For example, the CR antigen domain can comprise an anti-CD4 antibody (e.g., scFv or sdAb) specifically recognizing domain 1 of CD4. In some embodiments, the TCR subunit is selected from the group consisting of TCRα, TCRβ, TCRγ, TCRδ, CD3γ, CD3ε. In some embodiments, the CR transmembrane domain, the intracellular CR signaling domain, and the optional extracellular domain of a TCR subunit or a portion thereof are derived from the same TCR subunit. In some embodiments, the CR transmembrane domain, the intracellular CR signaling domain, and the optional extracellular domain of a TCR subunit or a portion thereof are derived from CD3ε. In some embodiments, the cTCR comprises the CR antigen binding domain fused to the N-terminus of a full length CD3ε. In some embodiments, the CD4 binding moiety and the bNAb moiety are linked in tandem. In some embodiments, the CD4 binding moiety is N-terminal to the bNAb moiety. In some embodiments, the CD4 binding moiety is C-terminal to the bNAb moiety. In some embodiments, the engineered immune cell further comprises a broadly neutralizing antibody (bNAb) or a nucleic acid encoding a bNAb, including VRC01, PGT121, 3BNC117, 10-1074, N6, VRC07, VRC07-523, eCD4-IG, 10E8, 10E8v4, PG9, PGDM 1400, PGT151, CAP256.25, 35O22, 8ANC195, and the like.

In some embodiments, there is provided a tandem anti-CCR5 bNAb cTCR comprising: i) a CR antigen binding domain comprising a CCR5 binding moiety (such as an anti-CCR5 antibody moiety, for example a scFv or sdAb) and a bNAb moiety (such as a scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising a tandem anti-CCR5 bNAb cTCR comprising: i) a CR antigen binding domain comprising a CCR5 binding moiety (such as an anti-CCR5 antibody moiety, for example a scFv or sdAb) and a bNAb moiety (such as a scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, there is provided an engineered immune cell comprising: a nucleic acid encoding a tandem anti-CCR5 bNAb cTCR comprising: i) a CR antigen binding domain comprising a CCR5 binding moiety (such as an anti-CCR5 antibody moiety, for example a scFv or sdAb) and a bNAb moiety (such as a scFv or sdAb); ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit; ii) an optional linker (such as a GS liner); iii) an optional extracellular domain of a TCR subunit or a portion thereof; iii) a CR transmembrane domain derived from a TCR subunit, and iv) an intracellular CR signaling domain derived from a TCR subunit. In some embodiments, the TCR subunit is selected from the group consisting of TCRα, TCRβ, TCRγ, TCRδ, CD3γ, CD3ε. In some embodiments, the CR transmembrane domain, the intracellular CR signaling domain, and the optional extracellular domain of a TCR subunit or a portion thereof are derived from the same TCR subunit. In some embodiments, the CR transmembrane domain, the intracellular CR signaling domain, and the optional extracellular domain of a TCR subunit or a portion thereof are derived from CD3ε. In some embodiments, the cTCR comprises the CR antigen binding domain fused to the N-terminus of a full length CD3ε. In some embodiments, the CCR5 binding moiety and the bNAb moiety are linked in tandem. In some embodiments, the CCR5 binding moiety is N-terminal to the bNAb moiety. In some embodiments, the CCR5 binding moiety is C-terminal to the bNAb moiety. In some embodiments, the engineered immune cell further comprises a broadly neutralizing antibody (bNAb) or a nucleic acid encoding a bNAb, including VRC01, PGT121, 3BNC117, 10-1074, N6, VRC07, VRC07-523, eCD4-IG, 10E8, 10E8v4, PG9, PGDM 1400, PGT151, CAP256.25, 35O22, 8ANC195, and the like.

### Chimeric Co-Stimulatory Receptor (CCOR)

The chimeric co-stimulatory receptor (CCOR) described herein specifically binds to a CCOR target antigen and allows for stimulating an immune cell on the surface of which it is functionally expressed upon target binding. The CCOR comprises a CCOR antigen binding domain that provides the target-binding specificity, a transmembrane domain, and a CCOR co-stimulatory domain that allows for stimulating the immune cell. The CCOR lacks a functional primary immune cell signaling sequence. In some embodiments, the CCOR lacks any primary immune cell signaling sequence. In some embodiments, the expression of the CCOR in the engineered immune cell is inducible. In some embodiments, the expression of the engineered immune cell is inducible upon signaling through the CR.

Examples of co-stimulatory immune cell signaling domains for use in the CCORs of the invention include the cytoplasmic sequences of co-receptors of the T cell receptor (TCR), which can act in concert with a CR to initiate signal transduction following CR engagement, as well as any derivative or variant of these sequences and any synthetic sequence that has the same functional capability.

The intracellular CCOR co-stimulatory domain can be a portion of the intracellular domain of a co-stimulatory molecule including, for example, CD28, 4-1BB (CD137), CD27, OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, and a ligand that specifically binds with CD83. In some embodiments, the intracellular CCOR co-stimulatory domain comprises a fragment of 4-1BB. In some embodiments, the intracellular CCOR co-stimulatory domain comprises a fragment of CD28 and a fragment of 4-1BB.

In some embodiments, the CCOR transmembrane domain comprises one or more transmembrane domains derived from, for example, CD28, CD3ε, CD3ζ, CD45, CD4, CD5, CD8, CD9, CD16, CD22, CD33, CD37, CD64, CD80, CD86, CD134, CD137, or CD154.

The CCOR antigen binding domain in some embodiments is selected from the group consisting of Fab, a Fab′, a (Fab′)₂, an Fv, a single chain Fv (scFv), a single domain antibody (sdAb), and a peptide ligand specifically binding to the CCOR target antigen. In some embodiments, the CCOR antigen binding domain specifically binds to a CCOR target antigen with a) an affinity that is at least about 10 (including for example at least about any of 10, 20, 30, 40, 50, 75, 100, 200, 300, 400, 500, 750, 1000 or more) times its binding affinity for other molecules; orb) a K_{d} no more than about 1/10 (such as no more than about any of 1/10, 1/20, 1/30, 1/40, 1/50, 1/75, 1/100, 1/200, 1/300, 1/400, 1/500, 1/750, 1/1000 or less) times its K_{d} for binding to other molecules. Binding affinity can be determined by methods known in the art, such as ELISA, fluorescence activated cell sorting (FACS) analysis, or radioimmunoprecipitation assay (RIA). K_{d} can be determined by methods known in the art, such as surface plasmon resonance (SPR) assay utilizing, for example, Biacore instruments, or kinetic exclusion assay (KinExA) utilizing, for example, Sapidyne instruments.

In some embodiments, the CCOR antigen binding domain is selected from the group consisting of Fab, a Fab′, a (Fab′)₂, an Fv, a single chain Fv (scFv), a single domain antibody (sdAb), and a peptide ligand specifically binding to the CR target antigen.

In some embodiments, the CCOR antigen binding domain is an antibody moiety. In some embodiments, the antibody moiety is monospecific. In some embodiments, the antibody moiety is multi-specific. In some embodiments, the antibody moiety is bispecific. In some embodiments, the antibody moiety is a tandem scFv, a diabody (Db), a single chain diabody (scDb), a dual-affinity retargeting (DART) antibody, a dual variable domain (DVD) antibody, a chemically cross-linked antibody, a heteromultimeric antibody, or a heteroconjugate antibody. In some embodiments, the antibody moiety is a scFv. In some embodiments, the antibody moiety is a single domain antibody (sdAb). In some embodiments, the antibody moiety is fully human, semi-synthetic with human antibody framework regions, or humanized.

The antibody moiety in some embodiments comprises specific CDR sequences derived from one or more antibody moieties (such as a monoclonal antibody) or certain variants of such sequences comprising one or more amino acid substitutions. In some embodiments, the amino acid substitutions in the variant sequences do not substantially reduce the ability of the antigen-binding domain to bind the target antigen. Alterations that substantially improve target antigen binding affinity or affect some other property, such as specificity and/or cross-reactivity with related variants of the target antigen, are also contemplated.

In some embodiments, the CCOR antigen binding domain binds the CCOR target antigen with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values).

### CR and CCOR Antigen-Binding Domains

The CR and CCOR described herein specifically recognize a target antigen selected from the group consisting of CD4, CCR5, and CXCR4. As discussed above, when the CR specifically recognizes CD4, the CCOR would specifically recognize CCR5 or CXCR4. Alternatively, when the CR specifically recognizes CCR5 or CXCR4, the CCOR would specifically recognize CD4. The target antigens and antigen binding domains are discussed in more detail in the section below, which are generally applicable to both the CR antigen binding domain (and CR target antigen) and the CCOR antigen binding domain (and CCOR target antigen).

In some embodiments, the target antigen is CCR5. CCR5 is a G protein-coupled receptor with seven transmembrane domains that belongs to the beta chemokine receptors family of integral membrane proteins. CCR5 is comprised of 352 amino acids and is approximately 41 kilodaltons (kDa).

CCR5 is predominantly expressed on cells of the immune system including T cells, macrophages, dendritic cells, and eosinophils, but it is also expressed in endothelium, epithelium, vascular smooth muscle and fibroblasts. Furthermore, it is expressed on microglia, neurons, and astrocytes found in the central nervous system. (See Barmania, F. and Pepper, M S. Applied & Translational Genomics (2013) 2 (2013):3-16.)

CCR5 is expressed on most HIV-I primary isolates and is critical for the establishment and maintenance of infection. HIV-1 isolates in early disease tend to use CCR5 as a co-receptor with CD4 for entry into CD4+ T cells. CCR5 has been shown to be upregulated on CD4+ T cells in HIV-infected individuals compared with uninfected controls (see Ostrowski, M A, et al. J. Immunol. (1998) 161(6):3195-3201). There is a measurable amount of inter-individual CCR5 surface expression variability.

In some embodiments, the target antigen is an extracellular domain variant of CCR5. In other embodiments, the target antigen is a transmembrane domain variant of CCR5. In yet other embodiments, the target antigen is an extracellular domain and transmembrane domain variant of CCR5 (For an analysis of several CCR5 variants and resultant HIV infectivity, see Zack Howard, O M, et al. J. Biol. Chem. (1999) 274(23):16228-16234.)

In some embodiments, the antigen binding domain is a ligand recognizing CCR5, a fragment thereof that is capable of recognizing CCR5. Ligands recognizing CCR5 include, but are not limited to, MIP-1α, MIP-1β, and RANTES. In some embodiments, the antigen binding domain is MIP-1α, also known as CCL3. MIP-1α is a cytokine that is involved in the recruitment and activation of polymorphonuclear leukocytes during acute inflammation. In some embodiments, the antigen binding domain is MIP-1β, also known as CCL4. MIP-1β is a chemoattractant for many immune cells including natural killer cells and monocytes. MIP1-β is produced by various cells of the immune system, including monocytes, T cells, B cells, and also by fibroblasts and endothelial and epithelial cells. In some embodiments, the antigen binding domain is a ligand is RANTES, also known as CCL5. RANTES recruits leukocytes to inflammatory sites, and it acts as a chemoattractant for various cells in the immune system, including T cells, basophils, and eosinophils. In yet other embodiments, the antigen binding domain is another ligand that binds to CCR5.

In some embodiments, the antigen binding domain is an antibody moiety recognizing CCR5, such as any of the antibody moieties described herein. Exemplary anti-CCR5 antibodies can be found at WO2006103100, which is specifically incorporated herein by reference.

In some embodiments, the target antigen is CXCR4. CXCR4, also known as C-X-C chemokine receptor type 4, is an alpha-chemokine receptor that binds the ligand SDF-1 and transmits intracellular signals through several different pathways resulting in an increase in calcium and/ or a decrease in cAMP levels. It is predominantly expressed on immune cells, including CD4+ T cells, but is also expressed on cells in the central nervous system. CXCR4 is a G protein-coupled receptor with seven transmembrane helices, and is comprised of 352 amino acids.

CXCR4 is one of several chemokine receptors that HIV isolates can use to infect CD4⁺ T cells. T cell tropic isolates of HIV can infect CD4+ T cells that are expressing CXCR4 on their surface. CXCR4 is a coreceptor for HIV entry into T cells and certain murine anti-CXCR4 antibodies have been demonstrated to be able to inhibit entry of HIV isolates into T cells (see Hou, T. et al. (1998) J. Immunol. 160:180-188; Carnec, X. et al. (2005) J. Virol. 79:1930-1938). CXCR4 can be used as a receptor by viruses for entry into the cell, and antibodies to CXCR4 have been used to inhibit cell entry of such viruses that use CXCR4 as a receptor. See WO2008060367 and WO2011098762.

CXCR4 is downregulated on CD4+ and CD8+ T cells and CD14+ monocytes in HIV-infected individuals compared to uninfected controls (see Ostrowski, M A, et al. J. Immunol. (1998) 161(6):3195-3201). HIV-1 isolates use CXCR4 as a coreceptor with CD4 for cell entry as the disease progresses; CXCR4 is not used as a coreceptor early in infection as is the case with CCR5.

In some embodiments, the antigen binding domain is a CXCR4 ligand, such as stromal cell-derived factor 1 (SDF-1, or CXCL12), or a fragment thereof that recognizes CXCR4. SDF-1 is strongly chemotactic for lymphocytes, and it is expressed in many tissues, including, but not limited to, the thymus, spleen, and bone marrow. In some embodiments, the antigen binding domain one of the seven isoforms of SDF-1. In some embodiments, the antigen binding domain is MIF, ubiquitin, or fragment thereof

In some embodiments, the antigen binding domain is an antibody moiety recognizing CXCR4, such as any of the antibody moieties described herein. Exemplary anti-CXCR4 antibodies can be found at WO2008060367 and WO2011098762, which are specifically incorporated herein by reference.

In some embodiments, the target antigen is CD4, also known as Cluster of Differentiation 4. CD4 is a glycoprotein found on the surface of immune cells, particularly CD4+, or helper, T cells. CD4 is a member of the immunoglobulin superfamily. CD4 is comprised of four extracellular immunoglobulin domains (D₁ to D₄). D₁ and D₃ show similarity to immunoglobulin variable domains, while D₂ and D₄ show similarity to immunoglobulin constant domains.

CD4 is an important cell-surface molecule required for HIV-1 entry and infection. HIV-1 entry is triggered by interaction of the viral envelope (Env) glycoprotein gp120 with domain 1 (D1) of the T-cell receptor CD4. As HIV infection progresses, greater numbers of CD4+ T cells are targeted and destroyed by the virus, resulting in an increasingly compromised immune system; CD4+ T cell count is therefore used as a proxy for the progression and stage of HIV/AIDS in an individual. Furthermore, HIV gene products Env, Vpu, and Nef, are involved in the downregulation of CD4 during HIV infection (see Tanaka, M., et al. Virology (2003) 311(2):316-325).

In some embodiments, the antigen binding domain specifically binds CD4 D1 or CD4 D2/D3 with a) an affinity that is at least about 10 (including for example at least about any of 10, 20, 30, 40, 50, 75, 100, 200, 300, 400, 500, 750, 1000 or more) times its binding affinity for other molecules; orb) a K_{d} no more than about 1/10 (such as no more than about any of 1/10, 1/20, 1/30, 1/40, 1/50, 1/75, 1/100, 1/200, 1/300, 1/400, 1/500, 1/750, 1/1000 or less) times its K_{d} for binding to other molecules. Binding affinity can be determined by methods known in the art, such as ELISA, fluorescence activated cell sorting (FACS) analysis, or radioimmunoprecipitation assay (RIA). K_{d} can be determined by methods known in the art, such as surface plasmon resonance (SPR) assay utilizing, for example, Biacore instruments, or kinetic exclusion assay (KinExA) utilizing, for example, Sapidyne instruments.

In some embodiments, the antigen binding domain is selected from the group consisting of Fab, a Fab′, a (Fab′)₂, an Fv, a single chain Fv (scFv), a single domain antibody (sdAb), and a peptide ligand specifically binding to CD4.

In some embodiments, the antigen binding domain is an antibody moiety. In some embodiments, the antibody moiety is monospecific. In some embodiments, the antibody moiety is multi-specific. In some embodiments, the antibody moiety is bispecific. In some embodiments, the antibody moiety is a tandem scFv, a diabody (Db), a single chain diabody (scDb), a dual-affinity retargeting (DART) antibody, a dual variable domain (DVD) antibody, a chemically cross-linked antibody, a heteromultimeric antibody, or a heteroconjugate antibody. In some embodiments, the antibody moiety is a scFv. In some embodiments, the antibody moiety is a single domain antibody (sdAb). In some embodiments, the antibody moiety is fully human, semi-synthetic with human antibody framework regions, or humanized.

The antibody moiety in some embodiments comprises specific CDR sequences derived from one or more antibody moieties (such as any of the specific antibodies disclosed herein) or certain variants of such sequences comprising one or more amino acid substitutions. In some embodiments, the amino acid substitutions in the variant sequences do not substantially reduce the ability of the antigen-binding domain to bind the target antigen. Alterations that substantially improve target antigen binding affinity or affect some other property, such as specificity and/or cross-reactivity with related variants of the target antigen, are also contemplated.

In some embodiments, the antigen binding domain binds to CD4 D1 or D2/3 with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values).

In some embodiments, the antigen binding domain binds to an epitope in D1 of CD4. In some embodiments, the antigen binding domain binds to an epitope that falls within any one or more of the following regions: amino acids 26-125, 26-46, 46-66, 66-86, 86-106, 106-125 of CD4, wherein the amino acid numbering is in accordance with SEQ ID NO. 1. In some embodiments, the antigen binding domain binds to an epitope that falls within any one or more of the following regions: amino acids 26-125, 26-46, 46-66, 66-86, 86-106, 106-125 of SEQ ID NO. 1. In some embodiments, the antigen binding domain binds to D1 of CD4 between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the CD4 is human CD4. In some embodiments, the antigen binding domain is derived from zanolimumab, for example as disclosed in WO1997013852. In some embodiments, the antigen binding domain competes for binding against zanolimumab. In some embodiments, the antigen binding domain binds to the same or overlapping epitope as that of zanolimumab.

In some embodiments, antigen binding domain binds to an epitope in D2 or D3 of CD4. In some embodiments, the antigen binding domain binds to an epitope that falls within any one or more of the following regions: amino acids 126-317, 126-203, 204-317, 126-146, 146-166, 166-186, 186-206, 206-226, 226-246, 246-266, 266-286, 286-306, and 306-317 of CD4, wherein the amino acid numbering is in accordance with SEQ ID NO. 1. In some embodiments, the antigen binding domain binds to an epitope that falls within any one or more of the following regions: amino acids 126-317, 126-203, 204-317, 126-146, 146-166, 166-186, 186-206, 206-226, 226-246, 246-266, 266-286, 286-306, and 306-317 of SEQ ID NO. 1. In some embodiments, the antigen binding domain binds to D2 or D3 of CD4 with a kd of between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the CD4 is human CD4. In some embodiments, the antigen binding domain is derived from ibalizumab or tregalizumab, for example as disclosed in US20130195881 and WO2004083247. In some embodiments, the antigen binding domain competes for binding against ibalizumab or tregalizumab. In some embodiments, the antigen binding domain binds to the same or overlapping epitope as that of ibalizumab or tregalizumab.

In some embodiments, the antigen binding domain is a ligand for CD4, or a fragment thereof capable of binding CD4. In some embodiments, the ligand for CD4 is IL-16, a pleiotropic cytokine that modulates T cell activation and inhibits HIV replication. In other embodiments, the ligand for CD4 is the class II major histocompatibility complex (MHC Class II). MHC Class II molecules are typically found on antigen presenting cells of the immune system, including B cells, dendritic cells, antigen presenting cells, mononuclear phagocytes, and thymic epithelial cells. In some embodiments, the MHC Class II ligand presents pathogenic peptides for presentation to CD4 for subsequent presentation to the immune system.

In some embodiments, the antigen binding domain is the envelope glycoprotein gp120 or a fragment thereof. Native gp120, encoded by the HIV env gene, is a 120 kDa glycoprotein found on the HIV viral envelope that plays an essential role in the attachment of HIV to target cells. gp120 binds to target cell CD4 and members of the chemokine receptor family, including CCR5, in order for there to be efficient HIV infection of a target cell. It has been shown that complexes of gp120 and CD4 interact specifically with CCR5 and inhibit the binding of natural CCR5 ligands like MIP-1α and MIP-1β (Wu, L., et al. (1996) Nature 384: 179-183).

In some embodiments when the antigen binding domain is gp120, the engineered immune cell does not comprise a CCOR. In some embodiments, the CR in the engineered immune cell comprises a CR co-stimulatory signaling domain.

In some embodiments, the antigen binding domain is a modified gp120 that does not bind one or more of CD4, CCR5, and CXCR4. For example, in some embodiments, the antigen binding domain is a modified gp120 that binds to CD4 but not CCR5 or CXCR4. In some embodiments, the antigen binding domain is a modified gp120 that binds to CCR5 but not CD4 or CXCR4. In some embodiments, the antigen binding domain is a modified gp120 that binds to CXCR4 but not CCR5 or CD4.

### Co-Receptor (“COR”)

In some embodiments, the engineered immune cells further comprise one or more co-receptors (“COR”).

In some embodiments, the COR facilitates the migration of the immune cell to follicles. In some embodiments, the COR facilitates the migration of the immune cell to the gut. In some embodiments, the COR facilitates the migration of the immune cells to the skin.

In some embodiments, the COR is CXCR5. In some embodiments, the COR is CCR9. In some embodiments, the COR is α4β7 (also referred to as integrin α4β7). In some embodiments, the engineered immune cell comprises two or more receptors selected from the group consisting of CXCR5, α4β7, and CCR9. In some embodiments, the engineered immune cell comprises both α4β7 and CCR9. In some embodiments, the engineered immune cell comprises CXCR5, α4β7, and CCR9.

CCR9, also known as C-C chemokine receptor type 9 (CCR9), is a member of the beta chemokine receptor family and mediates chemotaxis in response to its binding ligand, CCL25. CCR9 is predicted to be a seven transmembrane domain protein similar in structure to a G protein-coupled receptor. CCR9 is expressed on T cells in the thymus and small intestine, and it plays a role in regulating the development and migration of T lymphocytes (Uehara, S., et al. (2002) J. Immunol. 168(6):2811-2819). CCR9/CCL25 has been shown to direct immune cells to the small intestine (Pabst, O., et al. (2004). J. Exp. Med. 199(3):411). Co-expressing a CCR9 in the immune cells can thus direct the engineered immune cells to the gut. In some embodiments, a splicing variant of CCR9 is used.

α4β7, or lymphocyte Peyer patch adhesion molecule (LPAM), is an integrin that is expressed on lymphocytes and that is responsible for T-cell homing into gut-associated lymphoid tissue (Petrovic, A. et al. (2004) Blood 103(4):1542-1547). α4β7 is a heterodimer comprised of CD49d (the protein product of ITGA4, the gene encoding the α4 integrin subunit) and ITGB7 (the protein product of ITGB4, the gene encoding the β7 integrin subunit). In some embodiments, a splicing variant of α4 is incorporated into the α4β7 heterodimer. In some embodiments, a splicing variant of β7 is incorporated into the α4β7 heterodimer. In other embodiments, splicing variants of α4 and splicing variants of β7 are incorporated into the heterodimer. Co-expression of α4β7, alone or in combination of CCR9, can direct the engineered immune cells to the gut.

Although α4β7 and CCR9 both function in homing to the gut, they are not necessarily co-regulated. The vitamin A metabolite retinoic acid plays a role in the induction of expression of both CCR9 and α4β7. α4β7 expression, however, can be induced through other means, while CCR9 expression requires retinoic acid. Furthermore, colon-tropic T-cells express only α4β7 and not CCR9, showing that the two receptors are not always coexpressed or coregulated. (See Takeuchi, H., et al. J. Immunol. (2010) 185(9):5289-5299.)

In some embodiments, CCR9 and α4β7 function as CORs for targeting to the gut.

In some embodiments, the immune cell expresses CXCR5, also known as C-X-C chemokine receptor type 5. CXCR5 is a G protein-coupled receptor containing seven transmembrane domains that belongs to the CXC chemokine receptor family. CXCR5 and its ligand, the chemokine CXCL13, play a central role in trafficking lymphocytes to follicles within secondary lymphoid tissues, including lymph nodes and the spleen. (Bürkle, A. et al. (2007) Blood 110:3316-3325.) In particular, CXCR5 enables T cells to migrate to lymph node B cell zones in response to CXCL13 (Schaerli, P. et al. (2000) J. Exp. Med. 192(11):1553-1562.) When expressed in the immune cell, CXCR5 can function as a COR for targeting the engineered immune cells to follicles. In some embodiments, a splicing variant of CXCR5 is used.

In general, a non-naturally occurring variant of any of the CORs discussed above can be comprised/expressed in the engineered immune cells. These variants may, for example, contain one or more mutations, but nonetheless maintain some or more functions of the corresponding native receptors. For example, in some embodiments, the COR is a variant of a naturally occurring CCR9, α4β, or CXCR5, wherein the variant has an amino acid sequence that is at least about any of 90%, 95%, 96%, 97%, 98%, or 99% identical to a native CCR9, α4β, or CXCR5. In some embodiments, the COR is a variant of a naturally occurring CCR9, β4β, or CXCR5, wherein the variant comprises no more than about any of 1, 2, 3, 4, 5, 6, 7, 8, 9, or 10 amino acid substitutions as compared to that of a native CCR9, α4β, or CXCR5.

In some embodiments, the COR is a chemokine receptor. In some embodiments, the COR is an integrin. In some embodiments, the COR is selected from the group consisting of CCR1, CCR2, CCR3, CCR4, CCR5, CCR6, CCR7, CCR8, CCR9, CCR10, CXCR1, CXCR2, CXCR3, CXCR4, CXCR5, CXCR6, CX₃CR1, XCR1, ACKR1, ACKR2, ACKR3, ACKR4, CCRL2.

In some embodiments, the COR is not normally expressed in the immune cell from which the engineered immune cell is derived from. In some embodiments, the COR is expressed at low levels in the immune cell from which the engineered immune cell is derived from.

### Anti-HIV Antibodies

The engineered immune cells described herein in some embodiments further express (and secret) an anti-HIV antibody, such as a broadly neutralizing antibody (“bNAb”). In some embodiments when the CR is a tandem CR, the CR antigen binding domain may comprise an anti-HIV antibody moiety (such as bNAb moiety). The bNAb binds to the HIV envelop protein and blocks the virus binding to the host cell receptors. bNAb moiety described herein refers to an antibody or a fragment thereof that retains the broadly neutralizing antibody activity and/or binding specificity. In some embodiments, the bNAb moiety is a scFv. In some embodiments, the bNAb moiety is a sdAb.

bNAb were first discovered in elite controllers, who were infected with HIV, but could naturally control the virus infection without taking antiretroviral medicines. bNAbs are neutralizing antibodies which neutralize multiple HIV viral strains. bNAb target conserved epitopes of the virus, even if the virus undergoes mutations. The engineered immune cells described herein in some embodiments can secret a broadly neutralizing antibody to block HIV infection of new host cells.

In some embodiments, the bNAb specifically recognizes a viral epitope on MPER of gp41, V1V2 glycan, outerdomain of glycan, V3 glycan, or CD4 binding site. bNAb blocks the interaction of the virus envelop glycoprotein with CD4. Mascola and Haynes, Immunol. Rev. 2013 July; 254(1):225-44. Suitable bNAbs include, but are not limited to, VRC01, PGT-121, 3BNC117, 10-1074, N6, VRC07, VRC07-523, eCD4-IG, 10E8, 10E8v4, PG9, PGDM 1400, PGT151, CAP256.25, 35O22, 8ANC195. Science Translational Medicine 23 Dec. 2015: Vol. 7, Issue 319, pp. 319ra206; PLoS Pathog. 2013; 9(5):e1003342; Nature. 2015 Jun. 25; 522(7557):487-91; Nat Med. 2017 February; 23(2):185-191; Nature Immunology volume 19, pages 1179-1188 (2018). Other suitable broadly neutralizing antibodies can be found at, for example, Cohen et al., Current Opin. HIV AIDS, 2018 July; 13(4):366-373; Mascola and Haynes, Immunol. Rev. 2013 July; 254(1):225-44.

### Nucleic Acids

Also provided herein are nucleic acids (or a set of nucleic acids) encoding the CR, CCOR and/or COR described herein, as well as vectors comprising the nucleic acid(s).

The expression of the CR, CCOR and/or COR can be achieved by inserting the nucleic acid into an appropriate expression vector, such that the nucleic acid is operably linked to 5′ and/or 3′ regulatory elements, including for example a promoter (e.g., a lymphocyte-specific promoter) and a 3′ untranslated region (UTR). The vectors can be suitable for replication and integration in host cells. Typical cloning and expression vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to, a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Exemplary methods to confirm the presence of the heterologous nucleic acid in the mammalian cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

Exemplary methods to confirm the presence of the heterologous nucleic acid in the mammalian cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

In some embodiments, each of the one or more nucleic acid sequences is contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

For example, in some embodiments, the nucleic acid comprises a first nucleic acid sequence encoding the CR polypeptide chain, optionally a second nucleic acid encoding the CCOR polypeptide chain, optionally a third nucleic acid encoding a COR polypeptide chain, and optionally a fourth nucleic acid encoding a bNAb polypeptide. In some embodiments, the first nucleic acid sequence is contained in a first vector, the second nucleic acid sequence is contained in a second vector, the third nucleic acid sequence is contained in a third vector, and/or the fourth nucleic acid sequence is contained in a fourth vector. In some embodiments, the first and second nucleic acid sequences are contained in a first vector, and the third nucleic acid sequence and/or fourth nucleic acid sequence is contained in a second vector. In some embodiments, the first and third nucleic acid sequences are contained in a first vector, and the second nucleic acid sequence and/or fourth nucleic acid sequence is contained in a second vector. In some embodiments, the second and third nucleic acid sequences are contained in a first vector, and the first nucleic acid sequence and/or fourth nucleic acid sequence is contained in a second vector. In some embodiments, the first, second, third, and optionally fourth nucleic acid sequences are contained in the same vector. In some embodiments, the first, second, third, and optionally fourth nucleic acids can be connected to each other via a linker selected from the group consisting of an internal ribosomal entry site (IRES) and a nucleic acid encoding a self-cleaving 2A peptide (such as P2A, T2A, E2A, or F2A).

In some embodiments, the first nucleic acid sequence is under the control of a first promoter, the second nucleic acid sequence is under the control of a second promoter, the third nucleic acid sequence is under the control of a third promoter, and/or the fourth nucleic acid sequence is under the control of a fourth promoter. In some embodiments, some or all of the first, second, third, and/or fourth promoters have the same sequence. In some embodiments, some or all of the first, second, third, and optionally fourth promoters have different sequences. In some embodiments, some or all of the first, second, third, and optionally fourth nucleic acid sequences are expressed as a single transcript under the control of a single promoter in a multicistronic vector. In some embodiments, one or more of the promoters are inducible. In some embodiments, the third and/or fourth nucleic acid sequence is operably linked to an inducible promoter.

In some embodiments, some or all of the first, second, third, and optionally fourth nucleic acid sequences have similar (such as substantially or about the same) expression levels in an immune cell (such as a T cell). In some embodiments, some of the first, second, third, and optionally fourth nucleic acid sequences have expression levels in an immune cell (such as a T cell) that differ by at least about two (such as at least about any of 2, 3, 4, 5, or more) times. Expression can be determined at the mRNA or protein level. The level of mRNA expression can be determined by measuring the amount of mRNA transcribed from the nucleic acid using various well-known methods, including Northern blotting, quantitative RT-PCR, microarray analysis and the like. The level of protein expression can be measured by known methods including immunocytochemical staining, enzyme-linked immunosorbent assay (ELISA), western blot analysis, luminescent assays, mass spectrometry, high performance liquid chromatography, high-pressure liquid chromatography-tandem mass spectrometry, and the like.

Methods of introducing and expressing genes into a cell (such as immune cell) are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human, cells. Other viral vectors can be derived from lentivirus, poxviruses, herpes simplex virus 1, adenoviruses and adeno-associated viruses, and the like.

Chemical means for introducing a polynucleotide into a host cell (such as immune cell) include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Antibody Moieties

Various constructs described herein (such as the CR antigen binding domain or CCOR antigen binding domain) comprises an antibody moiety. In some embodiments, the antibody moiety comprises V_{H} and V_{L} domains, or variants thereof, from the monoclonal antibody. In some embodiments, the antibody moiety further comprises C_{H}1 and C_{L} domains, or variants thereof, from the monoclonal antibody. Monoclonal antibodies can be prepared, e.g., using hybridoma methods, such as those described by Kohler and Milstein, Nature, 256:495 (1975) and Sergeeva et al., Blood, 117(16):4262-4272.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest, or a complex comprising at least two molecules, such as a complex comprising a peptide and an MHC protein. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al. Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107: 220 (1980).

After the desired hybridoma cells are identified, the clones can be sub-cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub-clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, the antibody moiety comprises sequences from a clone selected from an antibody moiety library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The antibody moiety can be prepared using phage display to screen libraries for antibodies specific to the target antigen (such as a CD4, CCR5, or CXCR4 polypeptides). The library can be a human scFv phage display library having a diversity of at least one×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully-synthetic phage display library. In some embodiments, the library is a non-human phage display library.

Phage clones that bind to the target antigen with high affinity can be selected by iterative binding of phage to the target antigen, which is bound to a solid support (such as, for example, beads for solution panning or mammalian cells for cell panning), followed by removal of non-bound phage and by elution of specifically bound phage. In an example of solution panning, the target antigen can be biotinylated for immobilization to a solid support. The biotinylated target antigen is mixed with the phage library and a solid support, such as streptavidin-conjugated Dynabeads M-280, and then target antigen-phage-bead complexes are isolated. The bound phage clones are then eluted and used to infect an appropriate host cell, such as E. coli XL1-Blue, for expression and purification. In an example of cell panning, cells expressing CD4, CCR5, or CXCR4 are mixed with the phage library, after which the cells are collected and the bound clones are eluted and used to infect an appropriate host cell for expression and purification. The panning can be performed for multiple (such as about any of 2, 3, 4, 5, 6 or more) rounds with either solution panning, cell panning, or a combination of both, to enrich for phage clones binding specifically to the target antigen. Enriched phage clones can be tested for specific binding to the target antigen by any methods known in the art, including for example ELISA and FACS.

### Human and Humanized Antibody Moieties

The antibody moieties described herein can be human or humanized. Humanized forms of non-human (e.g., murine) antibody moieties are chimeric immunoglobulins, immunoglobulin chains, or fragments thereof (such as Fv, Fab, Fab′, F(ab′)₂, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody moiety nor in the imported CDR or framework sequences. In general, the humanized antibody moiety can comprise substantially all of at least one, and typically two, variable domains, in which all or substantially all of the CDR regions correspond to those of a non-human immunoglobulin, and all or substantially all of the FR regions are those of a human immunoglobulin consensus sequence. See, e.g., Jones et al., Nature, 321: 522-525 (1986); Riechmann et al., Nature, 332: 323-329 (1988); Presta, Curr. Op. Struct. Biol., 2:593-596 (1992).

Generally, a humanized antibody moiety has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody moiety. Accordingly, such “humanized” antibody moieties are antibody moieties (U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Additional Variants

In some embodiments, amino acid sequence variants of the antigen-binding domains provided herein are contemplated. For example, it may be desirable to improve the binding affinity and/or other biological properties of the antigen-binding domain. Amino acid sequence variants of an antigen-binding domain may be prepared by introducing appropriate modifications into the nucleotide sequence encoding the antigen-binding domain, or by peptide synthesis. Such modifications include, for example, deletions from, and/or insertions into and/or substitutions of residues within the amino acid sequences of the antigen-binding domain. Any combination of deletion, insertion, and substitution can be made to arrive at the final construct, provided that the final construct possesses the desired characteristics, e.g., antigen-binding.

In some embodiments, antigen-binding domain variants having one or more amino acid substitutions are provided. Sites of interest for substitutional mutagenesis include the HVRs and FRs of antibody moieties. Amino acid substitutions may be introduced into an antigen-binding domain of interest and the products screened for a desired activity, e.g., retained/improved antigen binding or decreased immunogenicity.

Conservative substitutions are shown in Table 4 below. Variant CORS discussed herein can also contain such conservative substitutions.

TABLE 4
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:

a. hydrophobic: Norleucine, Met, Ala, Val, Leu, Ile;

b. neutral hydrophilic: Cys, Ser, Thr, Asn, Gln;

c. acidic: Asp, Glu;

d. basic: His, Lys, Arg;

e. residues that influence chain orientation: Gly, Pro;

f. aromatic: Trp, Tyr, Phe.

Non-conservative substitutions will entail exchanging a member of one of these classes for another class.

An exemplary substitutional variant is an affinity matured antibody moiety, which may be conveniently generated, e.g., using phage display-based affinity maturation techniques. Briefly, one or more CDR residues are mutated and the variant antibody moieties displayed on phage and screened for a particular biological activity (e.g., binding affinity). Alterations (e.g., substitutions) may be made in HVRs, e.g., to improve antibody moiety affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).)

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody moiety variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody moiety to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antigen-binding domain that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antigen-binding domain with antigen is affected. Further substitutions may be introduced at the amino acid locations demonstrating functional sensitivity to the initial substitutions. Alternatively, or additionally, a crystal structure of an antigen-antigen-binding domain complex can be determined to identify contact points between the antigen-binding domain and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antigen-binding domain with an N-terminal methionyl residue. Other insertional variants of the antigen-binding domain include the fusion to the N- or C-terminus of the antigen-binding domain to an enzyme (e.g., for ADEPT) or a polypeptide which increases the serum half-life of the antigen-binding domain.

### Expression of Nucleic Acids

The heterologous nucleic acid described herein may be transiently or stably incorporated in the immune cells. In some embodiments, the heterologous nucleic acid is transiently expressed in the engineered immune cell. For example, the heterologous nucleic acid may be present in the nucleus of the engineered immune cell in an extrachromosomal array comprising the heterologous gene expression cassette. Heterologous nucleic acids may be introduced into the engineered mammalian using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection. In some embodiments, the heterologous nucleic acid is a DNA. In some embodiments, the heterologous nucleic acid is a RNA. In some embodiments, the heterologous nucleic acid is linear. In some embodiments, the heterologous nucleic acid is circular.

In some embodiments, the heterologous nucleic acid is present in the genome of the engineered immune cell. For example, the heterologous nucleic acid may be integrated into the genome of the immune cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN®), CRISPR/Cas9, and zinc-finger nucleases. In some embodiments, the heterologous nucleic acid is integrated in a specifically designed locus of the genome of the engineered immune cell. In some embodiments, the heterologous nucleic acid is integrated in an integration hotspot of the genome of the engineered immune cell. In some embodiments, the heterologous nucleic acid is integrated in a random locus of the genome of the engineered immune cell. In the cases that multiple copies of the heterologous nucleic acids are present in a single engineered immune cell, the heterologous nucleic acid may be integrated in a plurality of loci of the genome of the engineered immune cell.

The heterologous nucleic acids described herein (e.g., nucleic acids encoding the CR, CCOR, and COR) can be operably linked to a promoter. In some embodiments, the promoter is an endogenous promoter. For example, the nucleic acid (e.g., nucleic acid encoding the CR, CCOR, or COR) may be knocked-in to the genome of the engineered immune cell downstream of an endogenous promoter using any methods known in the art, such as CRISPR/Cas9 method. In some embodiments, the endogenous promoter is a promoter for an abundant protein, such as beta-actin. In some embodiments, the endogenous promoter is an inducible promoter, for example, inducible by an endogenous activation signal of the engineered immune cell. In some embodiments, wherein the engineered immune cell is a T cell, the promoter is a T cell activation-dependent promoter (such as an IL-2 promoter, an NFAT promoter, or an NFκB promoter).

In some embodiments, the promoter is a heterologous promoter.

In some embodiments, the heterologous nucleic acid (e.g., nucleic acid encoding the CR, CCOR, or COR) is operably linked to a constitutive promoter. In some embodiments, the heterologous nucleic acid (e.g., nucleic acid encoding the CR, CCOR, or COR) is operably linked to an inducible promoter. In some embodiments, a constitutive promoter is operably linked to the nucleic acid encoding a CR, and an inducible promoter is operably linked to a nucleic acid encoding a CCOR or COR. In some embodiments, a first inducible promoter is operably linked to a nucleic acid encoding a CR, and an second inducible promoter is operably linked to a nucleic acid encoding a CCOR, or vice versa. In some embodiments, a first inducible promoter is operably linked to a nucleic acid encoding a CR, and an second inducible promoter is operably linked to a nucleic acid encoding a COR, or vice versa. In some embodiments, a first inducible promoter is operably linked to a nucleic acid encoding a CCOR, and an second inducible promoter is operably linked to a nucleic acid encoding a COR, or vice versa. In some embodiments, the first inducible promoter is inducible by a first inducing condition, and the second inducible promoter is inducible by a second inducing condition. In some embodiments, the first inducing condition is the same as the second inducing condition. In some embodiments, the first inducible promoter and the second inducible promoter are induced simultaneously. In some embodiments, the first inducible promoter and the second inducible promoter are induced sequentially, for example, the first inducible promoter is induced prior to the second inducible promoter, or the first inducible promoter is induced after the second inducible promoter.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the promoter in the heterologous nucleic acid is a hEF1α promoter.

The inducible promoter can be induced by one or more conditions, such as a physical condition, microenvironment of the engineered immune cell, or the physiological state of the engineered immune cell, an inducer (i.e., an inducing agent), or a combination thereof. In some embodiments, the inducing condition does not induce the expression of endogenous genes in the engineered immune cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered immune cell.

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most mature system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a tumor antigen. In some embodiments, the polypeptide is expressed by the engineered immune cell. In some embodiments, the polypeptide is encoded by a nucleic acid in the heterologous nucleic acid. Many polypeptide inducers are also known in the art, and they may be suitable for use in the present invention. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present invention. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB* and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

In some embodiments, the promoter is a light-inducible promoter, and the inducing condition is light. Light inducible promoters for regulating gene expression in mammalian cells are also well-known in the art (see, for example, Science 332, 1565-1568 (2011); Nat. Methods 9, 266-269 (2012); Nature 500: 472-476 (2013); Nature Neuroscience 18:1202-1212 (2015)). Such gene regulation systems can be roughly put into two categories based on their regulations of (1) DNA binding or (2) recruitment of a transcriptional activation domain to a DNA bound protein. For instance, synthetic mammalian blue light controlled transcription system based on melanopsin which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10(3):196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the light-inducible promoters described herein may be used to drive expression of the therapeutic protein in the present invention.

In some embodiments, the promoter is a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and AP1. The immediate early genes comprise radiation responsive sequences in their promoter regions. Consensus sequences CC(A/T)₆GG have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, Jan. 20, 2015. Any of the immediate early gene promoters or any promoter comprising a serum response element or may be useful as a radiation inducible promoter to drive the expression of the therapeutic protein of the present invention.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Huang et al reported that after introduction of hsp70B-EGFP, hsp70B-TNFalpha and hsp70B-IL12 coding sequences, tumor cells expressed extremely high transgene expression upon heat treatment, while in the absence of heat treatment, the expression of transgenes were not detected. And tumor growth was delayed significantly in the IL12 transgene plus heat treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present invention.

In some embodiments, the promoter is inducible by a redox state. Exemplary promoters that are inducible by redox state include inducible promoter and hypoxia inducible promoters. For instance, Post DE et al developed hypoxia-inducible factor (HIF) responsive promoter which specifically and strongly induce transgene expression in HIF-active tumor cells (Gene Ther. 8: 1801-1807 (2001); Cancer Res. 67: 6872-6881 (2007)).

In some embodiments, the promoter is inducible by the physiological state, such as an endogenous activation signal, of the engineered immune cell. In some embodiments, wherein the engineered immune cell is a T cell, the promoter is a T cell activation-dependent promoter, which is inducible by the endogenous activation signal of the engineered T cell. In some embodiments, the engineered T cell is activated by an inducer, such as PMA, ionomycin, or phytohaemagglutinin. In some embodiments, the engineered T cell is activated by recognition of a tumor antigen on the tumor cells via an endogenous T cell receptor, or an engineered receptor (such as recombinant TCR, or CAR). In some embodiments, the engineered T cell is activated by blockade of an immune checkpoint, such as by the immunomodulator expressed by the engineered T cell or by a second engineered immune cell. In some embodiments, the T cell activation-dependent promoter is an IL-2 promoter. In some embodiments, the T cell activation-dependent promoter is an NFAT promoter. In some embodiments, the T cell activation-dependent promoter is a NFκB promoter.

Without being bound by any theory or hypothesis, IL-2 expression initiated by the gene transcription from IL-2 promoter is a major activity of T cell activation. Un-specific stimulation of human T cells by Phorbol 12-myristate 13-acetate (PMA), or ionomycin, or phytohaemagglutinin results in IL-2 secretion from stimulated T cells. IL-2 promoter was explored for activation-induced transgene expression in genetically engineered T-cells (Virology Journal 3:97 (2006)). We found that IL-2 promoter is efficient to initiate reporter gene expression in the presence of PMA/PHA-P activation in human T cell lines. T cell receptor stimulation initiates a cascade of intracellular reactions causing an increasing of cytosolic calcium concentrations and resulting in nuclear translation of both NFAT and NFκB. Members of Nuclear Factor of Activated T cells (NFAT) are Ca²⁺ dependent transcription factors mediating immune response in T lymphocytes. NFAT have been shown to be crucial for inducible interleukine-2 (IL-2) expression in activated T cells (Mol Cell Biol. 15(11):6299-310 (1995); Nature Reviews Immunology 5:472-484 (2005)). We found that NFAT promoter is efficient to initiate reporter gene expression in the presence of PMA/PHA-P activation in human T cell lines. Other pathways including nuclear factor kappa B (NFκB) can also be employed to control transgene expression via T cell activation.

### Immune Cells

Exemplary immune cells useful for the present invention include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lymphokine-activated killer (LAK) cells), B cells, Natural Killer (NK) cells, NKT cells, γδT cells, monocytes, macrophages, neutrophils, granulocytes, and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In the cases that the pharmaceutical composition comprises a plurality of engineered mammalian immune cells, the engineered mammalian immune cells can be a specific subpopulation of an immune cell type, a combination of subpopulations of an immune cell type, or a combination of two or more immune cell types. In some embodiments, the immune cell is present in a homogenous cell population. In some embodiments, the immune cell is present in a heterogeneous cell population that is enhanced in the immune cell. In some embodiments, the engineered immune cell is a lymphocyte. In some embodiments, the engineered immune cell is not a lymphocyte. In some embodiments, the engineered immune cell is suitable for adoptive immunotherapy. In some embodiments, the engineered immune cell is a PBMC. In some embodiments, the engineered immune cell is an immune cell derived from the PBMC. In some embodiments, the engineered immune cell is a T cell. In some embodiments, the engineered immune cell is a CD4⁺ T cell. In some embodiments, the engineered immune cell is a CD8⁺ T cell. In some embodiments, the engineered immune cell is a B cell. In some embodiments, the engineered immune cell is an NK cell.

### Preparation of Engineered Immune Cells

The immune cells expressing various constructs described herein (such as the CR, CCOR, and/or COR) can be generated by introducing one or more nucleic acids (including for example a lentiviral vector), such as nucleic acids encoding the CR, CCOR, and/or COR into the immune cell. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered immune cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art.

In some embodiments, the transduced or transfected mammalian cell is propagated ex vivo after introduction of the heterologous nucleic acid. In some embodiments, the transduced or transfected mammalian cell is cultured to propagate for at least about any of 1 day, 2 days, 3 days, 4 days, 5 days, 6 days, 7 days, 10 days, 12 days, or 14 days. In some embodiments, the transduced or transfected mammalian cell is cultured for no more than about any of 1 day, 2 days, 3 days, 4 days, 5 days, 6 days, 7 days, 10 days, 12 days, or 14 days. In some embodiments, the transduced or transfected mammalian cell is further evaluated or screened to select the engineered immune cell.

The introduction of the one or more nucleic acids into the immune cell can be accomplished using techniques known in the art. In some embodiments, the engineered immune cells (such as engineered T cells) are able to replicate in vivo, resulting in long-term persistence that can lead to sustained control of a disease associated with expression of the target antigen (such as viral infection).

In some embodiments, the invention relates to administering an engineered immune cell described herein for the treatment of a patient having or at risk of developing an infectious disease such as HIV. In some embodiments, autologous lymphocyte infusion is used in the treatment. Autologous PBMCs are collected from a patient in need of treatment and T cells are activated and expanded using the methods described herein and known in the art and then infused back into the patient.

In some embodiments, there is provided an engineered immune cell described herein for use in treating HIV. The cells can undergo robust in vivo expansion and can establish target antigen-specific memory cells that persist at high levels for an extended amount of time in blood and bone marrow. In some embodiments, the engineered immune cells infused into a patient can eliminate virally-infected cells. In some embodiments, the engineered immune cells infused into a patient can eliminate virally-infected cells.

Prior to expansion and genetic modification of the immune cells, a source of immune cells is obtained from a subject. Immune cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of immune cell lines available in the art may be used. In some embodiments of the present invention, immune cells can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed and the cells directly resuspended in culture media.

In some embodiments, immune cells (such as T cells) are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. Longer incubation times may be used to isolate T cells in any situation where there are few T cells as compared to other cell types. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD11b, CD 16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

Whether prior to or after genetic modification of the immune cells to express a nucleic acid (such as nucleic acid desirable CR, CCOR and/or COR), the immune cells can be activated and expanded.

In some embodiments, the immune cells (such as T cells) described herein are expanded by contact with a surface having attached thereto an agent that stimulates a CD3/TCR complex associated signal and a ligand that stimulates a co-stimulatory molecule on the surface of the T cells. In particular, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

### Genetic Modifications

In some embodiments, the engineered immune cell is a immune cells (such as T cell) modified to block or decrease the expression of CCR5. Modifications of cells to disrupt gene expression include any such techniques known in the art, including for example RNA interference (e.g., siRNA, shRNA, miRNA), gene editing (e.g., CRISPR- or TALEN-based gene knockout), and the like.

In some embodiments, engineered immune cells (such as T cells) with reduced expression of CCR5 are generated using the CRISPR/Cas system. For a review of the CRISPR/Cas system of gene editing, see for example Jian W & Marraffini L A, Annu. Rev. Microbiol. 69, 2015; Hsu P D et al., Cell, 157(6):1262-1278, 2014; and O'Connell M R et al., Nature 516: 263-266, 2014. In some embodiments, Engineered immune cells (such as engineered T cells) with reduced expression of one or both of the endogenous TCR chains of the T cell are generated using TALEN-based genome editing.

In some embodiments, the CCR5 gene (or TCR gene) is inactivated using CRISPR/Cas9 gene editing. CRISPR/Cas9 involves two main features: a short guide RNA (gRNA) and a CRISPR-associated endonuclease or Cas protein. The Cas protein is able to bind to the gRNA, which contains an engineered spacer that allows for directed targeting to, and subsequent knockout of, a gene of interest. Once targeted, the Cas protein cleaves the DNA target sequence, resulting in the knockout of the gene.

In some embodiments, the CCR5 gene (or TCR gene) is inactivated using transcription activator-like effector nuclease (TALEN®)-based genome editing. TALEN®-based genome editing involves the use of restriction enzymes that can be engineered for targeting to particular regions of DNA. A transcription activator-like effector (TALE) DNA-binding domain is fused to a DNA cleavage domain. The TALE is responsible for targeting the nuclease to the sequence of interest, and the cleavage domain (nuclease) is responsible for cleaving the DNA, resulting in the removal of that segment of DNA and subsequent knockout of the gene.

In some embodiments, the CCR5 gene (or TCR gene) is inactivated using zinc finger nuclease (ZFN) genome editing methods. Zinc finger nucleases are artificial restriction enzymes that are comprised of a zinc finger DNA-binding domain and a DNA-cleavage domain. ZFN DNA-binding domains can be engineered for targeting to particular regions of DNA. The DNA-cleavage domain is responsible for cleaving the DNA sequence of interest, resulting in the removal of that segment of DNA and subsequent knockout of the gene.

In some embodiments, the expression of the CCR5 gene (or TCR gene) is reduced by using small interference RNA (siRNA). siRNA molecules are 20-25 nucleotide long oligonucleotide duplexes that are complementary to messenger RNA (mRNA) transcripts from genes of interest. siRNAs target these mRNAs for destruction. Through targeting, siRNAs prevent mRNA transcripts from being translated, thereby preventing the protein from being produced by the cell.

In some embodiments, the expression of the CCR5 gene (or TCR gene) is reduced by using anti-sense oligonucleotides. Antisense oligonucleotides targeting mRNA are generally known in the art and used routinely for downregulating gene expressions. See Watts, J. and Corey, D (2012) J. Pathol. 226(2):365-379.)

### Enrichment of the Engineered Immune Cells

In some embodiments, there is provided a method of enriching a heterogeneous cell population for an engineered immune cell according to any of the engineered immune cells described herein.

A specific subpopulation of engineered immune cells (such as engineered T cells) that specifically bind to a target antigen and target ligand can be enriched for by positive selection techniques. For example, in some embodiments, engineered immune cells (such as engineered T cells) are enriched for by incubation with target antigen-conjugated beads and/or target ligand-conjugated beads for a time period sufficient for positive selection of the desired engineered immune cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of engineered immune cells present at low levels in the heterogeneous cell population, use of longer incubation times, such as 24 hours, can increase cell yield. Longer incubation times may be used to isolate engineered immune cells in any situation where there are few engineered immune cells as compared to other cell types. The skilled artisan would recognize that multiple rounds of selection can also be used in the context of this invention.

For isolation of a desired population of engineered immune cells by positive selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of engineered immune cells that may weakly express the CR, CCOR, and/or COR.

In some of any such embodiments described herein, enrichment results in minimal or substantially no exhaustion of the engineered immune cells. For example, in some embodiments, enrichment results in fewer than about 50% (such as fewer than about any of 45, 40, 35, 30, 25, 20, 15, 10, or 5%) of the engineered immune cells becoming exhausted. Immune cell exhaustion can be determined by any means known in the art, including any means described herein.

In some of any such embodiments described herein, enrichment results in minimal or substantially no terminal differentiation of the engineered immune cells. For example, in some embodiments, enrichment results in fewer than about 50% (such as fewer than about any of 45, 40, 35, 30, 25, 20, 15, 10, or 5%) of the engineered immune cells becoming terminally differentiated. Immune cell differentiation can be determined by any means known in the art, including any means described herein.

In some of any such embodiments described herein, enrichment results in minimal or substantially no internalization of CR, CCOR, and/or COR on the engineered immune cells. For example, in some embodiments, enrichment results in less than about 50% (such as less than about any of 45, 40, 35, 30, 25, 20, 15, 10, or 5%) of CR, CCOR, and/or COR on the engineered immune cells becoming internalized. Internalization of CR, CCOR, or COR on engineered immune cells can be determined by any means known in the art, including any means described herein.

In some of any such embodiments described herein, enrichment results in increased proliferation of the engineered immune cells. For example, in some embodiments, enrichment results in an increase of at least about 10% (such as at least about any of 20, 30, 40, 50, 60, 70, 80, 90, 100, 200, 300, 400, 500, 1000% or more) in the number of engineered immune cells following enrichment.

Thus, in some embodiments, there is provided a method of enriching a heterogeneous cell population for engineered immune cells expressing a CR that specifically binds to a CR target antigen and/or a CCOR that specifically binds to a CCOR target ligand comprising: a) contacting the heterogeneous cell population with a first molecule comprising the target antigen or one or more epitopes contained therein and/or a second molecule comprising the target ligand or one or more epitopes contained therein to form complexes comprising the engineered immune cell bound to the first molecule and/or complexes comprising the engineered immune cell bound to the second molecule; and b) separating the complexes from the heterogeneous cell population, thereby generating a cell population enriched for the engineered immune cells. In some embodiments, the first and/or second molecules are immobilized, individually, to a solid support. In some embodiments, the solid support is particulate (such as beads). In some embodiments, the solid support is a surface (such as the bottom of a well). In some embodiments, the first and/or second molecules are labeled, individually, with a tag. In some embodiments, the tag is a fluorescent molecule, an affinity tag, or a magnetic tag. In some embodiments, the method further comprises eluting the engineered immune cells from the first and/or second molecules and recovering the eluate.

In some embodiments, the immune cells or engineered immune cells are enriched for for CD4+ and/ or CD8+ cells, for example through the use of negative enrichment, whereby cell mixtures are purified using two-step purification methods involving both physical (column) and magnetic (MACS magnetic beads) purification steps (Gunzer, M. et al. (2001) J. Immunol. Methods 258(1-2):55-63). In other embodiments, populations of cells can be enriched for CD4+ and/ or CD8+ cells through the use of T cell enrichment columns specifically designed for the enrichment of CD4+ or CD8+ cells. In yet other embodiments, cell populations can be enriched for CD4+ cells through the use of commercially available kits. In some embodiments, the commercially available kit is the EasySep™ Human CD4+ T Cell Enrichment Kit (Stemcell Technologies™). In other embodiments, the commercially available kit is the MagniSort Mouse CD4+ T cell Enrichment Kit (Thermo Fisher Scientific). In yet other embodiments, the commercially available enrichment kit is one known to a person skilled in the art.

### Pharmaceutical Compositions

Also provided herein are engineered immune cell compositions (such as pharmaceutical compositions, also referred to herein as formulations) comprising an engineered immune cell (such as a T cell) described herein.

The composition may comprise a homogenous cell population comprising engineered immune cells of the same cell type and expressing the same CR and/or CCOR, or a heterogeneous cell population comprising a plurality of engineered immune cell populations comprising engineered immune cells of different cell types, expressing different CRs, different CCORs, and/or different CORs. The composition may further comprise cells that are not engineered immune cells.

Thus, in some embodiments, there is provided an engineered immune cell composition comprising a homogeneous cell population of engineered immune cells (such as engineered T cells) of the same cell type and expressing the same CR, CCOR, and/or COR. In some embodiments, the engineered immune cell is a T cell. In some embodiments, the engineered immune cell is selected from the group consisting of a cytotoxic T cell, a helper T cell, a natural killer T cell, and a suppressor T cell. In some embodiments, the engineered immune cell composition is a pharmaceutical composition.

In some embodiments, there is provided an engineered immune cell composition comprising a heterogeneous cell population comprising a plurality of engineered immune cell populations comprising engineered immune cells of different cell types, expressing different CRs, different CCORs, and/or different CORs.

In some embodiments, the pharmaceutical composition is suitable for administration to an individual, such as a human individual. In some embodiments, the pharmaceutical composition is suitable for injection. In some embodiments, the pharmaceutical composition is suitable for infusion. In some embodiments, the pharmaceutical composition is substantially free of cell culture medium. In some embodiments, the pharmaceutical composition is substantially free of endotoxins or allergenic proteins. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is free of mycoplasma, microbial agents, and/or communicable disease agents.

The pharmaceutical composition of the present applicant may comprise any number of the engineered immune cells. In some embodiments, the pharmaceutical composition comprises a single copy of the engineered immune cell. In some embodiments, the pharmaceutical composition comprises at least about any of 1, 10, 100, 1000, 10⁴, 10⁵, 10⁶, 10⁷, 10⁸ or more copies of the engineered immune cells. In some embodiments, the pharmaceutical composition comprises a single type of engineered immune cell. In some embodiments, the pharmaceutical composition comprises at least two types of engineered immune cells, wherein the different types of engineered immune cells differ by their cell sources, cell types, expressed therapeutic proteins, immunomodulators, and/or promoters, etc.

At various points during preparation of a composition, it can be necessary or beneficial to cryopreserve a cell. The terms “frozen/freezing” and “cryopreserved/cryopreserving” can be used interchangeably. Freezing includes freeze drying.

In particular embodiments, cells can be harvested from a culture medium, and washed and concentrated into a carrier in a therapeutically-effective amount. Exemplary carriers include saline, buffered saline, physiological saline, water, Hanks' solution, Ringer's solution, Nonnosol-R (Abbott Labs), Plasma-Lyte A(R) (Baxter Laboratories, Inc., Morton Grove, Ill.), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In compositions and formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

Also provided herein are nucleic acid compositions (such as pharmaceutical compositions, also referred to herein as formulations) comprising any of the nucleic acids encoding a CR, CCOR and/or COR described herein. In some embodiments, the nucleic acid composition is a pharmaceutical composition. In some embodiments, the nucleic acid composition further comprises any of an isotonizing agent, an excipient, a diluent, a thickener, a stabilizer, a buffer, and/or a preservative; and/or an aqueous vehicle, such as purified water, an aqueous sugar solution, a buffer solution, physiological saline, an aqueous polymer solution, or RNase free water. The amounts of such additives and aqueous vehicles to be added can be suitably selected according to the form of use of the nucleic acid composition.

The compositions and formulations disclosed herein can be prepared for administration by, for example, injection, infusion, perfusion, or lavage. The compositions and formulations can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

The formulations to be used for in vivo administration must be sterile. This is readily accomplished by, e.g., filtration through sterile filtration membranes.

### Excipient

The pharmaceutical compositions of the present invention are useful for therapeutic purposes. Thus, different from other compositions comprising engineered immune cells, such as production cells that express immunomodulators or other therapeutic proteins, the pharmaceutical compositions of the present invention comprises a pharmaceutically acceptable excipient suitable for administration to an individual.

Suitable pharmaceutically acceptable excipient may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, the pharmaceutically acceptable excipient comprises autologous serum. In some embodiments, the pharmaceutically acceptable excipient comprises human serum. In some embodiments, the pharmaceutically acceptable excipient is non-toxic, biocompatible, non-immunogenic, biodegradable, and can avoid recognition by the host's defense mechanism. The excipient may also contain adjuvants such as preserving stabilizing, wetting, emulsifying agents and the like. In some embodiments, the pharmaceutically acceptable excipient enhances the stability of the engineered immune cell or the immunomodulator or other therapeutic proteins secreted thereof. In some embodiments, the pharmaceutically acceptable excipient reduces aggregation of the immunomodulator or other therapeutic proteins secreted by the engineered immune cell. The final form may be sterile and may also be able to pass readily through an injection device such as a hollow needle. The proper viscosity may be achieved and maintained by the proper choice of excipients.

In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition can also be made to be isotonic with blood by the addition of a suitable tonicity modifier, such as glycerol.

In some embodiments, the pharmaceutical composition is suitable for administration to a human. In some embodiments, the pharmaceutical composition is suitable for administration to a human by parenteral administration. Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a condition requiring only the addition of the sterile liquid excipient methods of treatment, methods of administration, and dosage regimens described herein (i.e., water) for injection, immediately prior to use. In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the pharmaceutical composition is formulated for intravenous administration. In some embodiments, the pharmaceutical composition is formulated for subcutaneous administration. In some embodiments, the pharmaceutical composition is formulated for local administration to a tumor site. In some embodiments, the pharmaceutical composition is formulated for intratumoral injection.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the engineered mammalian immune cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the engineered immune cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the engineered immune cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### Methods of Treatment Using Engineered Immune Cells

The present application further provides methods of administering the engineered immune cells to treat diseases, including, but not limited to, infectious diseases, EBV positive T cell lymphoproliferative disorder, T-cell prolymphocytic leukemia, EBV-positive T cell lymphoproliferative disorders, adult T-cell leukemia/lymphoma, mycosis fungoides/sezary syndrome, primary cutaneous CD30-positive T-cell lymphoproliferative disorders, peripheral T-cell lymphoma (not otherwise specified), angioimmunoblastic T-cell lymphoma, and anaplastic large cell lymphoma, and autoimmune disease.

In some embodiments, autologous lymphocyte infusion is used in the treatment. Autologous PBMCs are collected from a patient in need of treatment and T cells are activated and expanded using the methods described herein and known in the art and then infused back into the patient.

The cells can undergo robust in vivo expansion and can establish CD4-specific memory cells that persist at high levels for an extended amount of time in blood and bone marrow. In some embodiments, the engineered immune cells infused into a patient can deplete cancer or virally-infected cells. In some embodiments, the engineered immune cells infused into a patient can eliminate cancer or virally-infected cells. Viral infection treatments can be evaluated, for example, by viral load, duration of survival, quality of life, protein expression and/or activity.

The engineered immune cells of the invention in some embodiments can be administered to individuals (e.g., mammals such as humans) to treat a cancer, for example CD4+ T cell lymphoma or T-cell leukemia. The present application thus in some embodiments provides a method for treating a cancer in an individual comprising administering to the individual an effective amount of a composition (such as a pharmaceutical composition) comprising engineered immune cells according to any one of the embodiments described herein. In some embodiments, cancer is T cell lymphoma.

In some embodiments, the methods of treating a cancer described herein further comprises administering to the individual a second anti-cancer agent. Suitable anti-cancer agents include, but are not limited to, CD70 targeting drugs, TRBC1, CD30 targeting drugs, CD37 targeting drugs, CCR4 targeting drugs, CHOP(cyclophosphamide, doxorubicin, vincristine and prednisone), CHOEP(cyclophosphamide, doxorubicin, vincristine, etoposide and prednisone), EPOCH (etoposide, vincristine, doxorubicin, cyclophosphamide and prednisone), Hyper-CVAD (cyclophosphamide, vincristine, doxorubicin, and dexamethasone), HDAC inhibitors, CD52 antibodyBelinostat, Bendamustine, BL-8040, Bortezomib, CPI-613, Mogamulizumab, Nelarabine, Nivolumab, Romidepsin and Ruxolitinib. In some embodiments, the second agent is an immune checkpoint inhibitor (e.g., an anti-CTLA4 antibody, an anti-PD1 antibody, or an anti-PD-L1 antibody). In some embodiments, the second anti-cancer agent is administered simultaneously with the engineered immune cells. In some embodiments, the second anti-cancer agent is administered sequentially with (e.g., prior to or after) the administration of the engineered immune cells. In some embodiments, the engineered immune cell compositions of the invention are administered in combination with a second, third, or fourth agent (including, e.g., an antineoplastic agent, a growth inhibitory agent, a cytotoxic agent, or a chemotherapeutic agent) to treat diseases or disorders involving target antigen expression.

The engineered immune cells of the invention can also be administered to individuals (e.g., mammals such as humans) to treat an infectious disease, for example HIV. The present application thus in some embodiments provides a method for treating an infectious disease in an individual comprising administering to the individual an effective amount of a composition (such as a pharmaceutical composition) comprising engineered immune cells according to any one of the embodiments described herein. In some embodiments, the viral infection is caused by a virus selected from, for example, Human T cell leukemia virus (HTLV) and HIV (Human immunodeficiency virus).

In some embodiments, methods of treating HIV are provided, which comprise administering any of the engineered immune cells described herein. There are two subtypes of HIV: HIV-1 and HIV-2. HIV-1 is the cause of the global pandemic and is a virus with both high virulence and high infectivity. HIV-2, however, is prevalent only in West Africa and is neither as virulent nor as infectious as HIV-1. The differences in virulence and infectivity between HIV-1 and HIV-2 infections may be rooted in the stronger immune response mounted against viral proteins in HIV-2 infections leading to more efficient control in affected individuals (Leligdowicz, A. et al. (2007) J. Clin. Invest. 117(10):3067-3074). This may also be a controlling reason for the global spread of HIV-1 and the limited geographic prevalence of HIV-2.

Although HIV-2 infections are better controlled than HIV-1 infections, HIV-2-affected individuals still benefit from treatment. In some embodiments, the engineered immune cells are used for treating HIV-1 infections. In other embodiments, the engineered immune cells are used for treating HIV-2 infections. In some embodiments, the engineered immune cells are used for treating HIV-1 and HIV-2 infections.

In some embodiments, the methods of treating an infectious disease described herein further comprises administering to the individual a second anti-infectious agent. Suitable anti-infectious agents include, but are not limited to, anti-retroviral drugs, broad neutralization antibodies, toll-like receptor agonists, latency reactivation agents, CCR5 antagonist, immune stimulators (e.g., TLR ligands), vaccines, nucleoside reverse transcriptase inhibitors, nucleotide reverse transcriptase inhibitors, non-nucleoside reverse transcriptase inhibitors, HIV protease inhibitors, and fusion inhibitors. In some embodiments, the second anti-infectious agent is administered simultaneously with the engineered immune cells. In some embodiments, the second anti-infectious agent is administered sequentially with (e.g., prior to or after) the administration of the engineered immune cells.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or environmentally or genetically prone to one or more of the diseases or disorders described herein (such as cancer or viral infection). In some embodiments, the individual has one or more risk factors associated with one or more diseases or disorders described herein.

In some embodiments, the pharmaceutical composition is administered at a dosage of at least about any of 10⁴, 10⁵, 10⁶, 10⁷, 10⁸, or 10⁹ cells/kg of body weight. In some embodiments, the pharmaceutical composition is administered at a dosage of any of about 10⁴ to about 10⁵, about 10⁵ to about 10⁶, about 10⁶ to about 10⁷, about 10⁷ to about10⁸, about 10⁸ to about 10⁹, about 10⁴ to about 10⁹, about 10⁴ to about 10⁶, about 10⁶ to about 10⁸, or about 10⁵ to about 10⁷ cells/kg of body weight.

In some embodiments, wherein more than one type of engineered immune cells are administered, the different types of engineered immune cells may be administered to the individual simultaneously, such as in a single composition, or sequentially in any suitable order.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly.

### Articles of Manufacture and Kits

In some embodiments of the invention, there is provided an article of manufacture containing materials useful for the treatment of an infectious disease such as viral infection (for example infection by HIV). The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an immune cell presenting on its surface a CR and a CCOR of the invention. The label or package insert indicates that the composition is used for treating the particular condition. The label or package insert will further comprise instructions for administering the engineered immune cell composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In other embodiments, the package insert indicates that the composition is used for treating a target antigen-positive viral infection (for example infection by HIV).

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a target antigen-positive disease or disorder described herein, optionally in combination with the articles of manufacture. Kits of the invention include one or more containers comprising an engineered immune cell composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

### Exemplary Embodiments

Embodiment 1. An engineered immune cell comprising: a) a chimeric receptor (CR) comprising: i) a CR antigen binding domain specifically recognizing a CR target antigen; ii) a CR transmembrane domain, and iii) an intracellular CR signaling domain; and b) a chimeric co-receptor (CCOR) comprising: i) a CCOR antigen binding domain specifically recognizing a CCOR target antigen; ii) a CCOR transmembrane domain; and iii) an intracellular CCOR co-stimulatory domain, wherein the CR target antigen is CCR5 or CXCR4 and the CCOR target antigen is CD4, or wherein the CR target antigen is CD4 and the CCOR target antigen is CCR5 or CXCR4.

Embodiment 2. An engineered immune cell comprising: a chimeric receptor (CR) comprising: i) a CR antigen binding domain specifically recognizing a CR target antigen; ii) a CR transmembrane domain, and iii) an intracellular CR signaling domain, wherein the CR target antigen is selected from the group consisting of CCR5, CXCR4 and CD4.

Embodiment 3. An engineered immune cell comprising: a chimeric receptor (CR) comprising: i) a CR antigen binding domain specifically recognizing a CR target antigen; ii) a CR transmembrane domain, and iii) an intracellular CR signaling domain, wherein the CR target antigen is selected from the group consisting of CCR5, CXCR4 and CD4, wherein the CCR5, CXCR4, or CD4 is in tandem with a broadly neutralizing antibody.

Embodiment 4. The engineered immune cell of embodiment 3, the broadly neutralizing antibody is VRC01, PGT121, 3BNC117 or 10-1074.

Embodiment 5. The engineered immune cell of any one of embodiments 1-4, further comprising one or more co-receptors (“COR”).

Embodiment 6. An engineered immune cell comprising: a) a first nucleic acid encoding a chimeric receptor (CR), wherein the CR comprises: i) a CR antigen binding domain specifically recognizing a CR target antigen; ii) a CR transmembrane domain, and iii) an intracellular CR signaling domain; and b) second nucleic acid encoding a chimeric co-receptor (CCOR), wherein the CCOR comprises: i) a CCOR antigen binding domain specifically recognizing a CCOR target antigen; ii) a CCOR transmembrane domain; and iii) an intracellular CCOR co-stimulatory signaling domain; wherein the CR target antigen is CCR5 or CXCR4 and the CCOR target antigen is CD4, or wherein the CR target antigen is CD4 and the CCOR target antigen is CCR5 or CXCR4.

Embodiment 7. An engineered immune cell comprising: a nucleic acid encoding a chimeric receptor (CR), wherein the CR comprises: i) a CR antigen binding domain specifically recognizing a CR target antigen; ii) a CR transmembrane domain, and iii) an intracellular CR signaling domain, wherein the CR target antigen is selected from the group consisting of CCR5, CXCR4 and CD4.

Embodiment 8. An engineered immune cell comprising: a nucleic acid encoding a chimeric receptor (CR), wherein the CR comprises: i) a CR antigen binding domain specifically recognizing a CR target antigen; ii) a CR transmembrane domain, and iii) an intracellular CR signaling domain, wherein the CR target antigen is selected from the group consisting of CCR5, CXCR4 and CD4, and wherein the CCR5, CXCR4, or CD4 is in tandem with a broadly neutralizing antibody.

Embodiment 9. The engineered immune cell of embodiment 8, the broadly neutralizing antibody is VRC01, PGT121, 3BNC117, 10-1074.

Embodiment 10. The engineered immune cell of embodiment 6-9, further comprising one or more nucleic acid(s) encoding one or more co-receptors (“COR”).

Embodiment 11. The engineered immune cell of any one of embodiments 1-10, wherein the CR is a chimeric antigen receptor (“CAR”).

Embodiment 12. The engineered immune cell of embodiment 11, wherein the CR transmembrane domain is derived from a molecule selected from the group consisting of CD8α, CD4, CD28, 4-1BB, CD80, CD86, CD152 and PD1.

Embodiment 13. The engineered immune cell of embodiment 12, wherein the CR transmembrane is derived from CD8α.

Embodiment 14. The engineered immune cell of embodiment 11, wherein the intracellular CR signaling domain is derived from CD3ζ, FcRγ, FcRβ, CD3γ, CD3δ, CD3ε, CD5, CD22, CD79a, CD79b, or CD66d.

Embodiment 15. The anti-CD4 immune cell receptor of embodiment 14, wherein the intracellular CR signaling domain is derived from CD3ζ.

Embodiment 16. The engineered immune cell of any one of embodiments 11-15, wherein the CR further comprises an intracellular CR co-stimulatory domain.

Embodiment 17. The engineered immune cell of embodiment 16, wherein the intracellular CR co-stimulatory signaling domain is derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof

Embodiment 18. The engineered immune cell of embodiment 17, wherein the intracellular CR co-stimulatory signaling domain comprises a cytoplasmic domain of 4-1-BB.

Embodiment 19. The engineered immune cell of any one of embodiments 11-18, further comprising a CR hinge domain located between the C-terminus of the CR antigen binding domain and the N-terminus of the CR transmembrane domain.

Embodiment 20. The engineered immune cell of embodiment 19, wherein the CR hinge domain is derived from CD8α.

Embodiment 21. The engineered immune cell of any one of embodiments 1-10, wherein the CR does not comprise an intracellular co-stimulatory domain.

Embodiment 22. The engineered immune cell of any one of embodiments 1-10, wherein the CR is a chimeric T cell receptor (“cTCR”).

Embodiment 23. The engineered immune cell of embodiment 22, wherein the CR transmembrane domain is derived from the transmembrane domain of a TCR subunit selected from the group consisting of TCRα, TCRβ, TCRγ, TCRδ, CD3γ, CD3ε, and CD3δ.

Embodiment 24. The engineered immune cell of embodiment 23, wherein the CR transmembrane domain is derived from the transmembrane domain of CD3ε.

Embodiment 25. The engineered immune cell of any one of embodiments 22-24, wherein the intracellular CR signaling domain is derived from the intracellular signaling domain of a TCR subunit selected from the group consisting of TCRα, TCRβ, TCRγ, TCRδ, CD3γ, CD3ε, and CD3δ.

Embodiment 26. The engineered immune cell of embodiment 25, wherein the intracellular CR signaling domain is derived from the intracellular signaling domain of CD3ε.

Embodiment 27. The engineered immune cell of any one of embodiments 22-26, wherein the CR transmembrane domain and intracellular CR signaling domain are derived from the same or different TCR subunit(s).

Embodiment 28. The engineered immune cell of any one of embodiments 22-27, wherein the CR further comprises a portion of an extracellular domain of a TCR subunit.

Embodiment 29. The engineered immune cell of any one of embodiments 22-28, wherein the CR comprises the CR antigen binding domain fused to the N-terminus of CD3ε.

Embodiment 30. The engineered immune cell of any one of embodiments 6-29, wherein the nucleic acid encoding the CR is under an inducible promoter.

Embodiment 31. The engineered immune cell of any one of embodiments 6-29, wherein the nucleic acid encoding the CR is constitutively expressed.

Embodiment 32. The engineered immune cell of any one of embodiments 6-31, wherein the nucleic acid encoding the CCOR and/or COR is under an inducible promoter.

Embodiment 33. The engineered immune cell of any one of embodiments 6-31, wherein the nucleic acid encoding the CCOR and/or COR is constitutively expressed.

Embodiment 34. The engineered immune cell of 32, wherein the nucleic acid encoding the CCOR and/or COR is inducible upon activation of the immune cell.

Embodiment 35. The engineered immune cell of any one of embodiments 6-34, wherein the first nucleic acid and the second nucleic acid are on the same vector.

Embodiment 36. The engineered immune cell of embodiment35, wherein the first nucleic acid and the second nucleic acid are under the control of the same promoter.

Embodiment 37. The engineered immune cell of any one of embodiments 6-3 1, wherein the first nucleic acid and the second nucleic acid are on different vectors.

Embodiment 38. The engineered immune cell of any one of embodiments 10-37, wherein one or more COR-encoding nucleic acids is on the same vector as the first nucleic acid.

Embodiment 39. The engineered immune cell of any one of embodiments 10-38, wherein one or more COR encoding nucleic acids is on the same vector as the second nucleic acid.

Embodiment 40. The engineered immune cell of embodiment 38 or 39, wherein the one or more COR encoding nucleic acid and the first nucleic acid or the second nucleic acid are under the control of the same promoter.

Embodiment 41. The engineered immune cell of any one of embodiments 1-40, wherein the CR target antigen is CD4.

Embodiment 42. The engineered immune cell of any one of embodiments 1, 5, 6, and 10-40, wherein the CCOR target antigen is CD4.

Embodiment 43. The engineered immune cell of any one of embodiments 1, 5, 6, and 10-40 and 42, wherein the CR target antigen is CCR5 or CXCR4 and the CCOR target antigen is CD4.

Embodiment 44. The engineered immune cell of any one of embodiments 1, 5, 6, and 10-41, wherein the CR target antigen is CD4 and the CCOR target antigen is CCR5 or CXCR4.

Embodiment 45. The engineered immune cell of embodiment 41-44, wherein the CR antigen binding domain or the CCOR antigen binding domain specifically recognizes domain 1 of CD4 (CD4 D1).

Embodiment 46. The engineered immune cell of any one of embodiments 5 and 10-45, wherein the one or more COR is selected from the group consisting of CXCR5, α4β7, and CCR9.

Embodiment 47. The engineered immune cell of embodiment 46, wherein the one or more COR is CXCR5.

Embodiment 48. The engineered immune cell of embodiment 46 or 47, wherein the one or more COR is α4β7.

Embodiment 49. The engineered immune cell of any one of embodiments 46-48, wherein the one or more COR is CCR9.

Embodiment 50. The engineered immune cell of any one of embodiments 46-49, wherein the one or more COR comprises both α4β7 and CCR9.

Embodiment 51. The engineered immune cell of any one of embodiments 1-50, wherein the engineered immune cell is modified to reduce or eliminate expression of CCR5 within the cell.

Embodiment 52. The engineered immune cell of any one of embodiments 1-51, wherein the engineered immune cell is modified to express an anti-HIV antibody.

Embodiment 53. The engineered immune cell of embodiment 52, wherein the anti-HIV antibody is a broadly neutralizing antibody.

Embodiment 54. The engineered immune cell of embodiment 53, wherein the broadly neutralizing antibody is VRC01, PGT121, 3BNC117 10-1074.

Embodiment 55. The engineered immune cell of any one of embodiments 1-54, wherein the CR antigen binding domain is selected from the group consisting of Fab, a Fab′, a (Fab′)₂, an Fv, a single chain Fv (scFv), a single domain antibody (sdAb), and a peptide ligand specifically binding to the CR target antigen.

Embodiment 56. The engineered immune cell of embodiment 55, wherein the CR antigen binding domain is scFv or sdAb.

Embodiment 57. The engineered immune cell of any one of embodiments 1, 5, 6, and 10-56, wherein the CCOR antigen binding domain is selected from the group consisting of Fab, a Fab′, a (Fab′)₂, an Fv, a single chain Fv (scFv), a single domain antibody (sdAb), and a peptide ligand specifically binding to the CCOR target antigen.

Embodiment 58. The engineered immune cell of embodiment 57, wherein the CCOR antigen binding domain is scFv or sdAb.

Embodiment 59. The engineered immune cell of any one of embodiments 1-58, wherein the CCOR co-stimulatory domain is selected from the group consisting of a co-stimulatory domain of one or more of CD28, 4-1BB (CD137), CD27, OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, and a ligand that specifically binds with CD83.

Embodiment 60. The engineered immune cell of any one of embodiments 1-59, wherein the engineered immune cell is selected from the group consisting of a cytotoxic T cell, a helper T cell, a natural killer cell, a γδT cell and a natural killer T cell.

Embodiment 61. The engineered immune cell of embodiment 60, wherein the engineered immune cell is a cytotoxic T cell.

Embodiment 62. A pharmaceutical composition comprising the engineered immune cell of any one of embodiments 1-61 and a pharmaceutically acceptable carrier.

Embodiment 63. The pharmaceutical composition of embodiment 62, wherein the pharmaceutical composition comprises at least two different types of engineered immune cells according to any one of embodiments 1-61.

Embodiment 64. A method of treating an infectious disease in an individual, comprising administering to the individual an effective amount of a pharmaceutical composition of embodiment 62 or 63.

Embodiment 65. The method of embodiment 64, wherein the infectious disease is an infection by a virus selected from the group consisting of HIV and HTLV.

Embodiment 66. The method of embodiment 65, wherein the infectious disease is HIV.

Embodiment 67. The method of embodiment 64-66, further comprising administering to the individual a second anti-infectious agent.

Embodiment 68. The method of embodiment 67, wherein the infectious agent is selected from the group consisting of anti-retroviral drugs, broad neutralization antibodies, toll-like receptor agonists, latency reactivation agents, CCR5 antagonist, immune stimulator, and a vaccine.

Embodiment 69. A method of treating a cancer in an individual, comprising administering to the individual an effective amount of a pharmaceutical composition of embodiment 62 or 63.

Embodiment 70. The method of embodiment 69, wherein the cancer is T cell lymphoma.

Embodiment 71. The method of embodiment 69 or 70, further comprising administering to the individual a second anti-cancer agent.

Embodiment 72. The method of embodiment 71, wherein the second anti-cancer agent is selected from the group consisting of CD70 targeting drugs, TRBC1, CD30 targeting drugs, CD37 targeting drugs and CCR4 targeting drugs.

Embodiment 73. The method of any one of embodiments 64-72, wherein the individual is a human.

Embodiment 74. A method of making an engineered immune cell of any one of embodiments 1-61, comprising: a) providing a population of immune cells; b) introducing into the population of immune cells a first nucleic acid encoding the CR.

Embodiment 75. The method of embodiment 74, further comprising: c) introducing into the population of immune cells a second nucleic acid encoding the CCOR.

Embodiment 76. The method of embodiment 75, wherein the first nucleic acid and the second nucleic acid are introduced into the cells simultaneously.

Embodiment 77. The method of embodiment 75, wherein the first nucleic acid and the second nucleic are introduced into the cells sequentially.

Embodiment 78. The method of any one of embodiments 74-77, further comprising introducing into the population of immune cells one or more nucleic acids encoding one or more CORs.

Embodiment 79. The method of any one of embodiments 74-78, wherein the first nucleic acid, the second nucleic acid, and/or the COR encoding nucleic acids are introduced into the cell via a viral vector.

Embodiment 80. The method of any one of embodiments 74-79, further comprising introducing into the population of immune cells a nucleic acid encoding a broadly neutralizing antibody (bNAb) or a HIV fusion inhibition peptide.

Embodiment 81. The method of any one of embodiments 74-80, further comprising inactivating the CCR5 gene in the cell.

Embodiment 82. The method of embodiment 81, wherein the CCRS gene is inactivated by using the method selected from the group consisting of: CRISPR/Cas9, TALEN, ZFN, siRNA, and antisense RNA.

Embodiment 83. The method of any one of embodiments 74-82, further comprising obtaining the population of immune cells from the peripheral blood of an individual.

Embodiment 84. The method of embodiment 83, wherein the population of immune cells are further enriched for CD4+ cells.

Embodiment 85. The method of embodiment 83 or 84, wherein the population of immune cells are further enriched for CD8+ cells.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1: Expression of CR and CCOR in Primary T Cells and Other Mammalian Cells

The lentiviral vectors carrying nucleic acids encoding a CR, CCOR, and optionally COR driven by a constitutive promoter hEF1α, a doxycycline inducible promoter (such as TetOn), an NFAT-dependent inducible promoter, or a heat inducible promoter (such as human heat shock protein 70 promoter, HSP70p) are designed and prepared. Primary human peripheral blood mononuclear cells (PBMC) are prepared by density gradient centrifugation of peripheral blood from healthy donors. Human primary T cells are purified from PBMCs using magnetic bead isolation. Human T cells are transduced with the lentiviral vectors and are expanded ex vivo for a couple of days. The expression of the receptors can be detected using methods known in the art. The bioactivity of the transduced T cells can be assessed through an in vitro target cell killing and other in vitro assays and in vivo animal models.

### Example 2. Materials and Methods

Cells. 293T cells were maintained in DMEM+10% FBS. Cryo-preserved human peripheral blood mononuclear cells (PBMC) were purchased from Hemacare. T cells were isolated from PBMC by T cell isolation kit (Miltenyi) and activated by T Cell Activation/Expansion Kit (Miltenyi). T cells were maintained in AIM-V media+5% Fetal Bovine Serum (FBS)+300 IU/ml IL-2.

Plasmids and lentivirus production. Chimeric antigen receptor (CAR) gene or eTCR gene were controlled by an EF1α promoter in pLVX vectors. The genes were synthesized and cloned into vectors by Genscript. 293T cells were transfected with the CAR or eTCR transfer plasmid, a 2^{nd} generation lentiviral packaging plasmid and a VSV-G envelop coding plasmid. Plasmids were transfected into 293T cells by 10% polyethylenimine (PEI) reagent. Virus supernatant was harvested at 48 hours and 72 hours post transfection. The supernatant was filtered through 0.45 μm sterile filter to remove cell debris. Viruses were concentrated by PEG method, aliquoted, and stored in −80° C.

CAR-T construction. Pan T cells were enriched from PBMC by pan T cell isolation kit (Miltenyi Biotech) and activated for 48 hours by T Cell Activation/Expansion Kit (Miltenyi Biotech). Activated T cells were incubated with the lentivirus in the presence of 8 μg/ml Polybrene and were spinoculated at room temperature at 1000 g for 1 hour. Cells were expanded in AIM-V media+5% Fetal Bovine Serum (FBS)+300 IU/ml recombinant human IL-2. The transduction efficiency was determined by flow cytometry as the CAR+ or eTCR+ percentage 4 days post transduction.

Cytotoxicity assay. Target cells were labeled with 2.5 μM CFSE in PBS for 5 min at room temperature before the reaction was stopped by the addition of 1/10 volume FBS. Cells were washed twice and were resuspended in culture media. Effector cells and target cells were co-cultured at desired Effector:Target ratios (E:T ratio) for 24 hours. Killing of CFSE labeled target cells were examined by flow cytometry.

SHIV infection assays. CD4 T cells were purified and activated in vitro for 4 days before they were infected with SHIVSF162P3 viruses. The cells were used as target cells at day 12 post infection. UNT cells, anti-CD4 CAR-T cells, anti-CCR5 CAR-T cells and tandem anti-CD4/anti-CCR5 cells were used as effector cells. The effector and target cells were co-cultured at 0.5:1 and 2:1 ratio for 72 hours. The virus positive cell rate was detected by p27 intracellular staining.

Flow cytometry. Anti-human CD3, CD4, CD8, CCR5 and p27 monoclonal antibodies were purchased from Biolegend and BD Bioscience. Goat anti-Human IgG, F(ab′)₂ polyclonal antibody was purchased from Jackson ImmunoResearch. Cells were resuspended in DPBS+2% FBS+1 mM EDTA for flow staining. Data were collected in BD FACSCelesta flow cytometer and were analyzed by Flowjo software (TreeStar).

HTRF assay. Target cells were co-cultured with effector cells for 24 hours. Cell culture media was harvested and IFNγ concentration was detected by HTRF assay (Cisbio). The assay was performed following the manufacturer's protocol. Briefly, 16 μl of samples were mixed with 4 μl of pre-mixed anti-IFNγ antibody in the assay plate. The plate was incubated at dark at room temperature overnight. The signal was detected by PheraStar microplate reader.

QPCR assay. Target T cells were infected with HIV pseudoviruses which also expressed enhanced green fluorescent protein (EGFP). Target cells were co-cultured with effector cells at 1:1 ratio for 24 hours. Cells were harvested and the genomic DNA was collected for QPCR. The integrated DNA copies were detected by detecting EGFP copies in the cell genomic DNA.

CAR-T treatment on lymphoma mouse model. NCG mice were implanted with HH cutaneous T cell lymphoma cells by subcutaneous injection. The mice were separated to 3 groups for treatment when the tumor size reached 120 mm³. Control UNT cells and CAR-T cells were resuspended in HBSS buffer. Cells were inoculated to the mice by tail-vein injection. One group of mice received 400 μl HBSS as control. One group of mice received 25 million UNT cells, and the last group of mice received 5 million CAR+ anti-CD4 CAR-T cells. Mice were observed, tumor size and body weight were recorded till the end of the experiment. The mice were sacrificed when the tumor size reached 2000 mm³.

CAR-T in vivo test in humanized mouse model. Neonatal NCG mice were transplanted with human hematopoietic stem cells to reconstitute the mice with human immune cells and were named as HIS mice. HIS mice were treated with anti-CD4, anti-CCR5 or tandem anti-CD4/anti-CCR5 CAR-T cells. And the presence of CD4+/CCR5+ cells were tested during the experiment.

### Example 3. Evaluation of CAR T Cells

This example describes evaluation of various CAR constructs exemplified in the present application.

### Construction of Chimeric Antigen Receptor (CAR) Expression Vector.

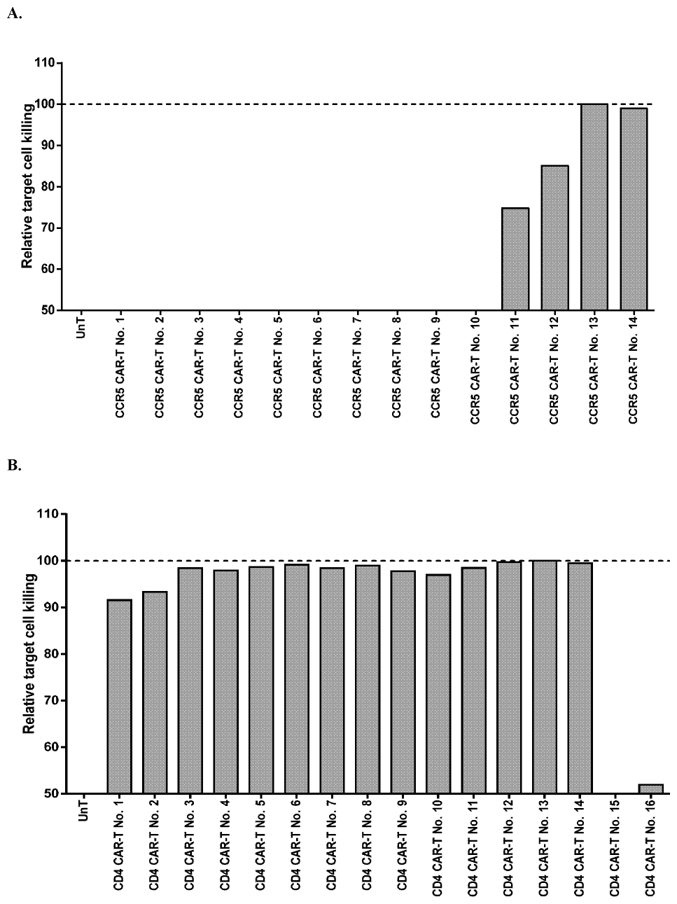

FIGS. 6A and 6B show schematic representations of CAR constructs containing anti-CD4 or anti-CCR5 or anti-CXCR4 scFv or sdAb (FIG. 6A) or anti-CD4 and anti-CCR5 scFv or sdAb linked in tandem (FIG. 6B). The CARs are composed of an antigen recognition domain, a hinge, a transmembrane domain, an intracellular co-stimulatory domain 4-1BB and a CD3ζ intracellular domain. The antigen recognition domain could be an scFv, a sdAb, two tandem scFv connected by a linker, or two tandem sdAb connected by a linker. The scFv or sdAb could recognize any of human CD4 or human CCR5 or human CXCR4. The linker can be (GGGGS)n, where n could be any number from 2 to 5. The hinge displayed here is part of the extracellular domain of human CD8 (SEQ ID NO. 2). The transmembrane domain is from human CD8 transmembrane domain (SEQ ID NO. 3). The intracellular domain is from human 4-1BB intracellular region (SEQ ID NO. 4) and CD3 signaling transduction domain (SEQ ID NO. 5). The CAR constructs used in the present example have the following sequences:

Anti-CD4 scFv-CD8 hinge-CD8 TM-4-1BB-CD3ζ (anti-CD4 CAR): SEQ ID NO. 11, No. 49-64.

Anti-CCR5 scFv-CD8 hinge-CD8 TM-4-1BB-CD3ζ (anti-CCR5 CAR): SEQ ID NO. 12, NO. 35-48.

Anti-CXCR4 scFv-CD8 hinge-CD8 TM-4-1BB-CD3ζ (anti-CXCR4 CAR): SEQ ID NO. 65-72.

Anti-CD4 scFv-anti-CCR5 scFv-CD8 hinge-CD8 TM-4-1BB-CD3ζ (tandem anti-CD4-anti-CCR5 CAR): SEQ ID NO. 13

Anti-CD4 scFv-CD8 hinge-CD8 TM-4-1BB-CD3ζ-P2A-CXCR5 (anti-CD4 CAR with CXCR5): SEQ ID NO. 14

Anti-CCR5 scFv-CD8 hinge-CD8 TM-4-1BB-CD3ζ-P2A-CXCR5 (anti-CCR5 CAR with CXCR5): SEQ ID NO. 15

Anti-CD4 scFv-anti-CCR5 scFv-CD8 hinge-CD8 TM-4-1BB-CD3ζ-P2A-CXCR5 (tandem anti-CD4-anti-CCR5 CAR with CXCR5): SEQ ID NO. 16

Anti-CD4 scFv-CD8 hinge-CD8 TM-4-1BB-CD3ζ-P2A-CXCR5-P2A-VRC01 (anti-CD4 CAR with CXCR5 and VRC01): SEQ ID NO. 17

Anti-CCR5 scFv-CD8 hinge-CD8 TM-4-1BB-CD3ζ-P2A-CXCR5-P2A-VRC01 (anti-CCR5 CAR with CXCR5 and VRC01): SEQ ID NO. 18

Anti-CD4 scFv-anti-CCR5 scFv-CD8 hinge-CD8 TM-4-1BB-CD3ζ-P2A-CXCR5-P2A-VRC01 (tandem anti-CD4-anti-CCR5 CAR with CXCR5 and VRC01): SEQ ID NO. 19

### CAR Cell Construction and Phenotyping.

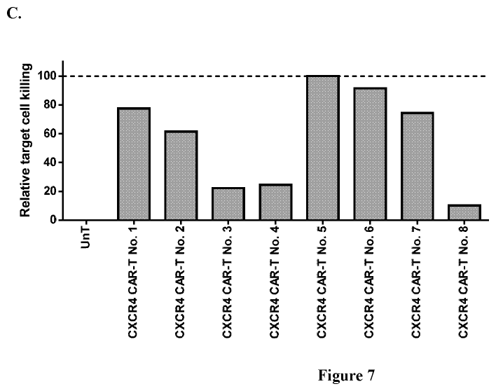

CAR-T cells were generated from T cells isolated from human peripheral mononuclear cells (PBMCs). T cells from PBMCs were enriched and activated in vitro before they were transduced with lentiviruses encoding the CAR genes. CD8+ T cells have target cell-killing function upon activation by secreting cytotoxic factors, perforin and granzyme B. The cytotoxic function of CD8 T cells is critical for the adaptive immune system to clear infected cells and to surveillance the intrinsic aberrant cell transformation. The target cell killing function is also the most important function of the artificially engineered CAR-T cell. To examine the cytotoxic function of engineered CAR-T cells, CAR-T cells were co-cultured with pan T cells at desired ratios. CAR-T cells thereof were named as effector cells (E), while pan T cells were named as target cells (T). Target cells were labeled with CFSE to be distinguished from the effector cells. Target and effector cells were co-cultured for 24 hours before they were harvested for flow cytometry. CFSE-labeled target cell percentage was recorded to reflect the cytotoxic effect. Un-transduced T cells (UNT) cells were used as negative control for the CAR-T cells. At one week post the CAR-T transduction, their key function, i.e. target cell killing ability, were tested during the initial screening. FIG. 7 shows the quantitative screening results of anti-CCR5 CAR-T, anti-CD4 CAR-T and anti-CXCR4 CAR-T cells. Among the 14 anti-CCR5 CAR-Ts, No. 13 had the best target killing effect. Among the 16 anti-CD4 CAR-Ts, No. 13 was the most efficient one. Among the 8 anti-CXCR4 CAR-Ts, No. 5 has the best target killing effect. The anti-CD4 CAR-T No.13 was renamed as CD4 CAR-T (SEQ ID NO. 11) and the scFv sequence was used in all the following anti-CD4 designs. The anti-CCR5 CAR-T No. 13 was renamed as anti-CCR5 CAR-T (SEQ ID NO. 12) and its scFv sequence was used in the complex designs.

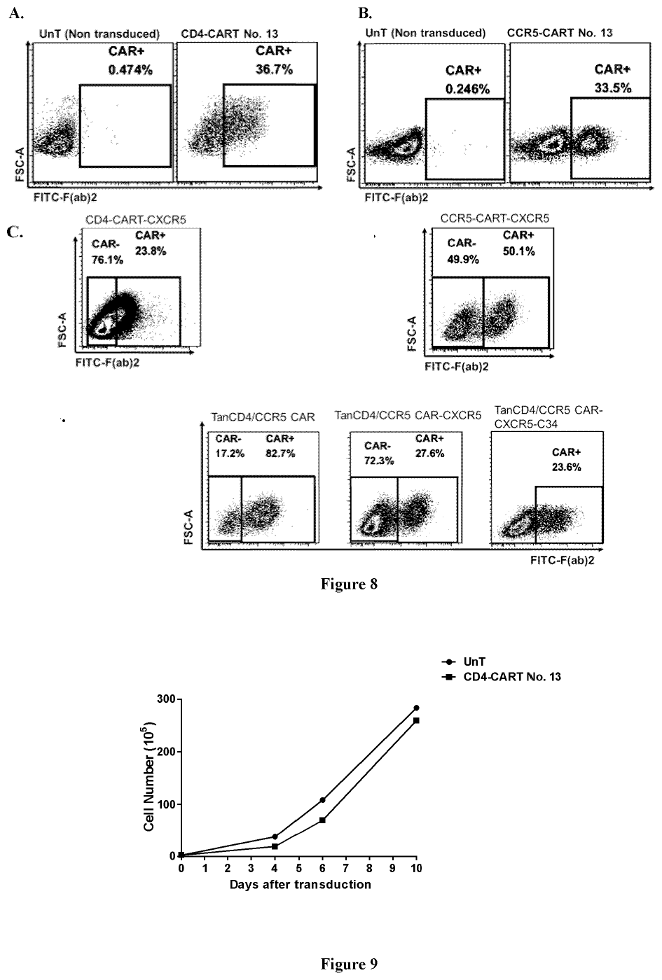

Though the CAR structures were similar to each other, not all chimeric antigen receptors could be transduced equally to the T cells. Goat anti-human IgG, F(ab′)₂ antibodies were used to detect the percentage of CAR+ T cells by flow cytometry. FIG. 8A-8E shows the CAR+% cells 4 days post the transduction.

CAR-T cells were cultured in AIM-V media+5% FBS+300 IU/ml IL-2. Cells were transferred to larger wells if confluent. Fresh complete media were supplied to support the cell expansion. Cell numbers and viability were recorded at day 0, day 4, day 6 and day 10 post transduction. The expansion of anti-CD4 CAR-T NO.13 is shown in FIG. 9.

CAR-T Cytotoxicity Assay

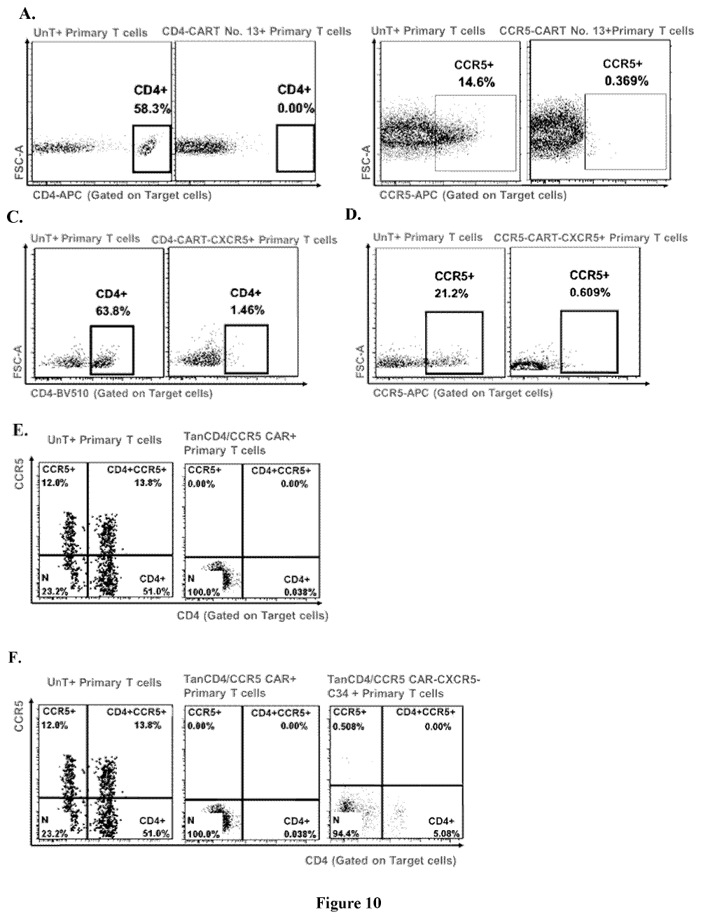

FIGS. 10A, 10B and 10E depict the flow cytometry result of the cytotoxic effect of CAR-T cells. As shown in FIG. 10A, there were 58% of CD4+ T cells in the target cells co-cultured with control UNT cells, but the CD4+ population decreased to 0% when the target cells were co-cultured with anti-CD4 CAR-T No.13 cells, suggesting a strong killing effect of the anti-CD4 CAR-T No.13 toward their targets. In FIG. 10B, the CCR5+ population was ˜15% when the target cells were co-cultured with UNT, but the population was reduced to less than 1% when the target cells were co-cultured with anti-CCR5 CAR-T No.13 cells, suggesting the anti-CCR5 CAR-T No.13 cells were very effective. FIG. 10E shows the effect of tandem anti-CD4/anti-CCR5-CART. The tandem CART cells not only eliminated the CD4 single positive cells and CCR5 single positive cells, but also efficiently eliminated the CD4/CCR5 double positive population.

### CAR-T Cytokine Profiling

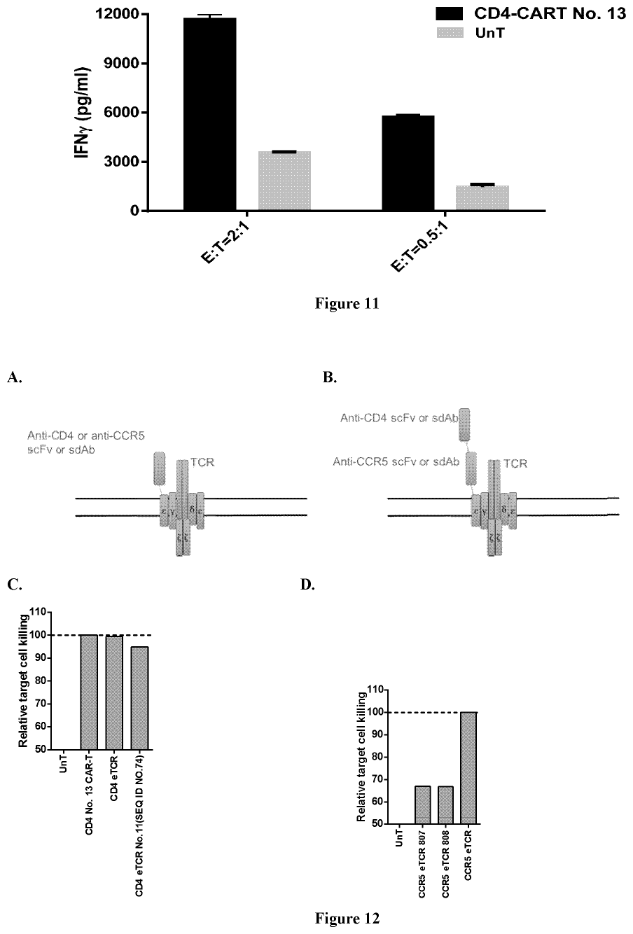

Besides perforin and granzyme B, CD8+ T cells also secret cytokines IFNγ and TNFα upon activation. These pro-inflammatory cytokines are important CD8+ T cell function indicators, but they may also play roles in the side effect of adoptive T cell therapy, cytokine release syndrome. The production of IFNγ was detected by HTRF assays in vitro. Day 6 supernatant post transduction were collected and the cytokine level were measured. FIG. 11 shows the production of theses cytokines by anti-CD4 CAR-T No.13 cells.

### Example 4. Evaluation of eTCR T Cells

This example describes evaluation of various chimeric TCR constructs exemplified in the present application.

### Construction of Anti-CD4-eTCR and Anti-CCR5-eTCR Expression Vector

T cell receptor α and β chain forms a complex with CD3 ε/δ/Γ/ζ chains. TCR recognizes antigens presented by MHC, leading to the phosphorylation of CD3 ζ chain and subsequent signal transduction to downstream pathways inside the cells to activate T cells. Natural T cell activation relies on the TCR-MHC interaction, thus is MHC dependent. This chimeric TCRs described herein modified the TCR complex to activate the T cells in an MHC-independent manner. The modification is on the CD3ε, the full name of which is T-cell surface glycoprotein CD3 epsilon chain (SEQ ID NO. 6). CD3ε sequence is available on public databases, such as Uniprot and NCBI Genebank. The sequence listed in SEQ ID NO. 6 is from Uniprot ID P07766. This chimeric TCR is name as eTCR hereby.

The eTCR gene is expressed by a lentivirus vector and is controlled by an EF1α promoter. CD3ε signal peptide sequence (SEQ ID NO. 7) is followed by the antigen recognition sequence. Linker (G4S)₃ sequence was added between the antigen recognition sequence and CD3ε sequence (SEQ ID NO. 8). The DNA sequences were optimized and de novo synthesized in Genscript and cloned into the pLVX lentivirus vector by Gateway Cloning. The eTCR constructs used in the present example has the following sequences:

Anti-CD4-CD3ε (anti-CD4 eTCR): SEQ ID NO. 20, 73

Anti-CCR5-CD3ε (anti-CCR5 eTCR): SEQ ID NO. 21, 74-76

Anti-CD4-CD3ε-P2A-CXCR5 (anti-CD4 eTCR with CXCR5): SEQ ID NO. 22

Anti-CCR5-CD3ε-P2A-CXCR5 (anti-CCR5 eTCR with CXCR5): SEQ ID NO. 23

Anti-CD4-anti-CCR5-CD3ε-P2A-CXCR5 (tandem antiCD4-anti-CCR5 eTCR with CXCR5): SEQ ID NO. 24

Anti-CD4-anti-CCR5-CD3ε-P2A-CXCR5-P2A-VRC01 (tandem antiCD4-antiCCR5 eTCR with CXCR5 and VRC01): SEQ ID NO. 25

FIG. 12A and FIG. 12B show schematic representations of exemplary eTCRs containing anti-CD4 or anti-CCR5 scFv or sdAb (FIG. 12A), or anti-CD4 and anti-CCR5 scFv or sdAb linked in tandem (FIG. 12B). An scFv, an sdAb, two tandem scFv or two tandem sdAbs are linked to the CD3ε chain by (G4S)₃ linkers.

The target cell killing effect was assessed as the key determinant for whether to further develop the eTCR design. To examine the target cell killing effect of engineered eTCR-T cells, eTCR-T cells were co-cultured with pan T cells at desired ratios. eTCR-T cells thereof were named as effector cells (E), while pan T cells were named as target cells (T). Target cells were labeled with CFSE to be distinguished from effector cells. Target and effector cells were co-cultured for 24 hours before they were harvested for flow cytometry. CFSE-labeled target cell percentage were recorded to reflect the cytotoxic effect. Un-transduced T cells (UNT) cells were used as negative control for the eTCR-T cells. FIG. 12C quantitatively compared the target cell killing effect of CD4 CAR-T No. 13, CD4 eTCR, CD4 eTCR No. 11. The CD4 eTCR No. 11 has an antigen binding region from Ibalizumab antibody sequence. The CD4 eTCR No. 11's target cell killing capability is less optimal than CD4 eTCR.

FIG. 12D showed the result of three screened anti-CCR5 eTCR constructs. Among the three anti-CCR5 constructs, CCR5 eTCR had the best target cell killing effect and was further studied.

eTCR-T Cell Phenotyping

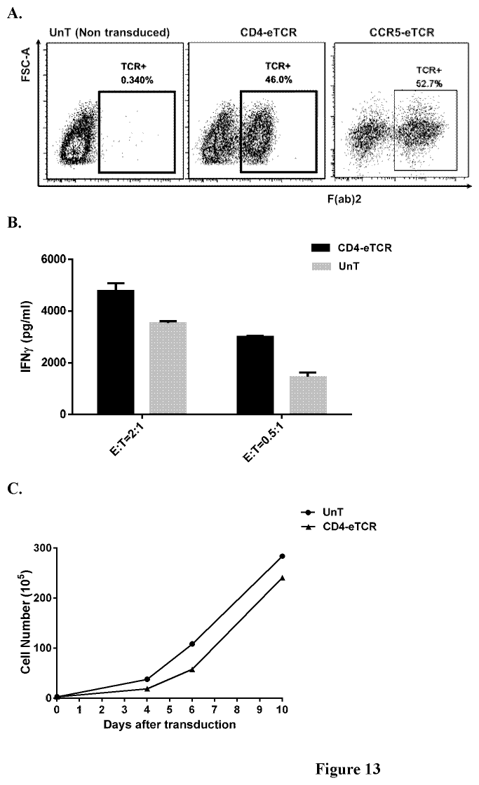

Like CAR-T cells, eTCR cells were generated by transducing activated T cells with eTCR coding lentiviruses. Not all T cells can be transduced at the same efficiency. Goat anti-human IgG, F(ab′)₂ antibodies were used to detect the percentage of eTCR+ T cells by flow cytometry. FIG. 13A shows the eTCR+% cells 4 days post the transduction.

eTCR-T Cell Expansion

eTCR-T cells were cultured in AIM-V media+5% FBS+300 IU/ml IL-2. Cells were expanded to larger wells if confluent and were supplied with fresh complete media to make sure the cells are always in ideal culture condition. Cell numbers and viability were recorded at day 0, day 4, day 6 and day 10 post transduction. The expansion of eTCR-T cells is showed in FIG. 13C.

eTCR-T Cytotoxicity

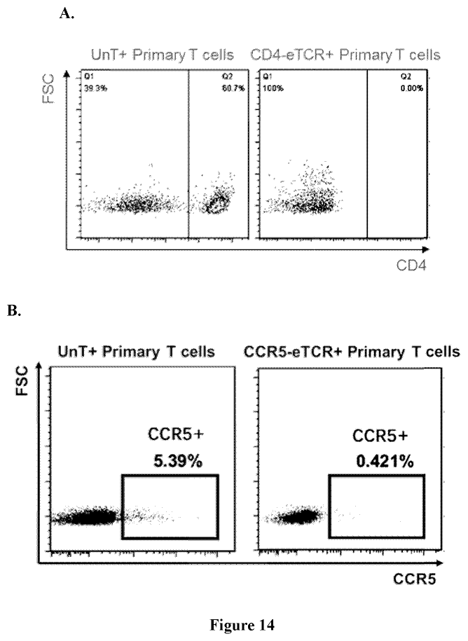

FIG. 14 shows the representative flow cytometry results of the eTCR-T mediated target cell killing. FIG. 14A depicts the flow cytometry result of the cytotoxic effect of anti-CD4 eTCR T cells that anti-CD4 eTCR-T cells could completely deplete the CD4+ population in target cells. Anti-CCR5 eTCR-T cells also killed most of the CCR5+ population as shown in FIG. 14B.

eTCR-T Cytokine Profiling

The production of IFNγ were detected by HTRF assays in vitro. eTCR-T cells were co-cultured with target pan T cells at 2:1 and 0.5:1 ratio for 24 hours and the supernatant was collected. FIG. 13B shows the production of IFNγ cytokine by the anti-CD4 eTCR-T cells. UNT cells were used as control. The IFNγ cytokine secreted by eTCR-T cells were only slightly higher than the UNT control cells.

### Example 5. CXCR5-Expressing T Cells

Lymph node is an important secondary lymphoid organ. B cells circulate in the peripheral blood and enter lymph node B cell follicles for their maturation, where they undergo isotype switching and affinity maturation process with the help of germinal center dendritic cells and follicle T helper cells. B cells, dendritic cells and follicle T cells express CXCR5, which is a receptor for CXCL13. CXCL13 is at high level in germinal center where it is produced by germinal center stromal cells and dendritic cells. CD8+ T cells is usually very rare in B cell follicles. Immunohistology staining showed high level of HIV virus hiding inside follicles, suggesting germinal center is a major HIV reservoir. It was reported that there were increased CD8+ T cells in germinal centers on elite HIV controllers. These CD8+ T cells co-express CXCR5 and express high level of cytotoxic effector factors and contribute to the virus control. Certain embodiments of the present application comprises co-expressing CXCR5 on CAR-T cells or eTCR-T cells.

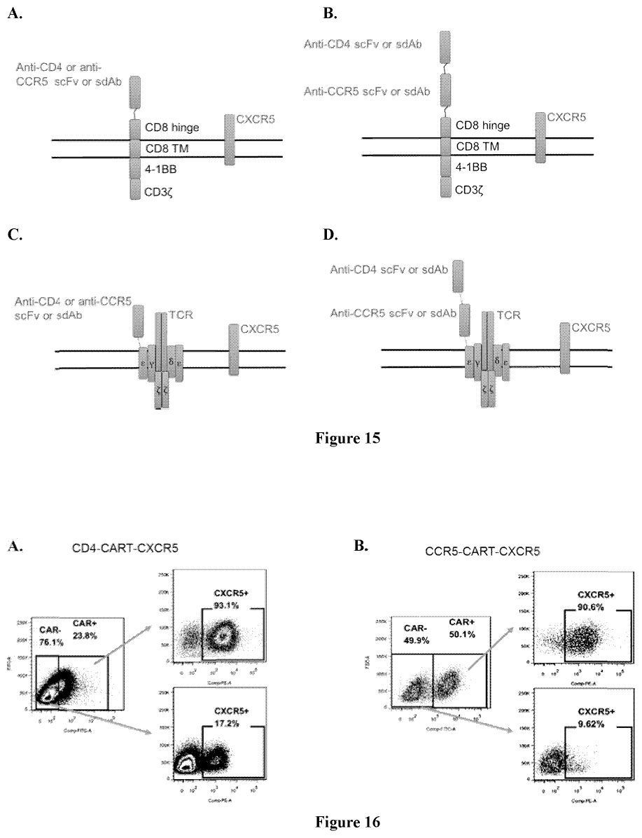

FIG. 15 illustrates the engineered T cells with CAR or eTCR and also express CXCR5. CXCR5 was linked to CAR or eTCR gene by a P2A linker. The antigen recognition region could be either anti-CD4, anti-CCR5 or tandem anti-CD4/anti-CCR5. It could be either scFv or sdAb. Anti-CXCR5 antibody was used to detect the CXCR5 expression.

The CAR and eTCR constructs used in this experiments are the same as above. CXCR5 has the following sequence: SEQ ID NO. 9. FIG. 16A depicts the expression of CXCR5 on the transduced anti-CD4 CAR-T cells, and FIG. 16B depicts the expression of CXCR5 on the anti-CCR5 CAR-T cells. As shown in the figures, over 90% of CAR+ cells also express high level of CXCR5.

CAR-T cells expressing CXCR5 could eliminate their target cells as efficient as those CAR-T cells without CXCR5 as shown in FIG. 10C and FIG. 10D. The CAR-T co-expressing CXCR5 cells were used as effector cells and were cultured with CFSE labeled pan T cells for 24 hours. In FIG. 10C, there were 63.8% CD4+ T cells when target cells were cultured with UNT control cells, and the population dropped to 1.46% when the target cells were co-cultured with anti-CD4 CAR-T cells co-expressing CXCR5. The CCR5+% was 21.2% in the UNT sample, while in the anti-CCR5 CAR-CXCR5 sample, the percentage was reduced to 0.609%, as shown in FIG. 10D.

FIG. 10F describes the cytotoxic effect of anti-CD4/anti-CCR5 tandem CAR-CXCR5-C34 T cells. The anti-CD4/anti-CCR5 tandem CAR-CXCR5-C34 T cells efficiently eliminated the CD4/CCR5 double positive populations and spared some of the CD4 or CCR5 single positive population, which will increase the safety in some disease circumstance.

### Example 6. Broadly Neutralizing Antibody-Expressing T Cells

Among HIV infected population, a small percentage of people can naturally control the virus infection without taking anti-retrovirus medicines. Studies found that they can generate robust antibody response against the virus infection. These antibodies were broadly neutralizing antibodies, as they recognize relatively conserved regions in HIV glycoprotein GP160 and can neutralize various subtypes of HIV. Broadly neutralizing antibodies have been tested in SHIV infected rhesus monkeys and can control the viral load for a median 21 weeks. Certain embodiments of the present application comprises co-expressing a broadly neutralizing antibody in CAR-T cells or eTCR-T cells. The CAR-T cells and eTCR-T cells can secret the broadly neutralizing antibody to block HIV infection of new host cells.

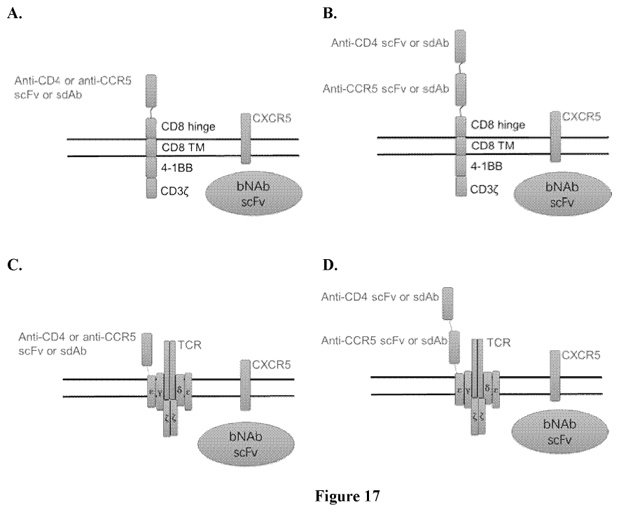

FIG. 17 depicts the engineered T cells expressing anti-CD4 or anti-CCR5 or tandem anti-CD4/anti-CCR5 CAR or eTCR, chemokine receptor CXCR5 and also a broadly neutralizing antibody (bNAb). The coding sequence was cloned into pLVX lenti-viral vector. The chimeric gene transcription was controlled by EF1α promoter. A P2A sequence was added after the CAR or eTCR sequence. Human CXCR5 sequence was added behind the P2A sequence. A bNAb with human IL2 signal peptide (SEQ ID NO. 34) was linked to CXCR5 by another P2A.

The CAR and eTCR constructs as well as CXCR5 used in this experiment are the same as above. The VRC01 has the following sequence: SEQ ID NO. 10. T cells were transduced with CAR-T lentiviruses encoding a His tag labeled VRC01 (VRC01-6His). The culture supernatant was harvested at day 8 post transduction. The VRC01 concentration in the supernatant was detected by ELISA using the anti-His tag antibody. The detected concentration was ˜40 ng/ml in the supernatant.

### Example 7. Functional Applications of CAR T Cells

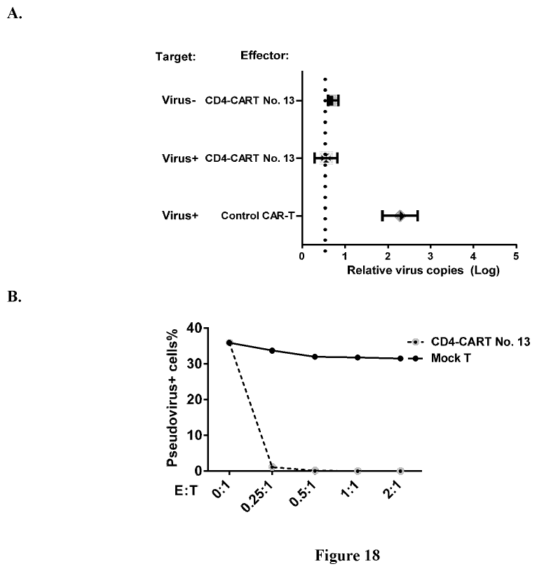

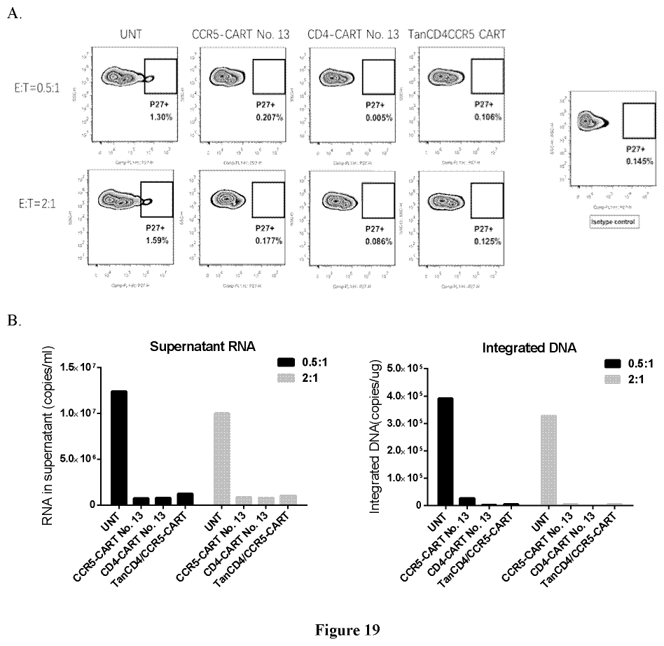

Anti-CD4 CAR-T cells kill CD4+ T cells, which are the major host cells for HIV infection. To illustrate whether these cells can eliminate viruses, CD4+ T cells were infected with HIV pseudoviruses in vitro. CAR-T cells were co-cultured with the infected cells. As shown in FIG. 18A, CAR-T cells greatly reduced the viral load in comparison to a control CAR-T which targets a B cell marker CD19 (SEQ ID NO. 77). Cells without virus infection was used as detection control. In FIG. 18B, T cells were infected with EGFP encoding HIV pseudoviruses that any cell successfully infected with the pseudoviruses became EGFP+. These cells were used as target cells and were co-cultured with anti-CD4 CAR-T No.13 cells or UNT cells. Different E:T ratios were used. The cells were co-cultured for 24 hours and the cells were harvested for detecting the EGFP percentage by flow cytometry. The EGFP+ rate decreased as the E:T ratio increased. The percentage of the pseudovirus+ cells dropped to near 0 when the E:T ratio reached 1:1 and above. In FIG. 19A, CAR-T constructs were incubated with SHIV infected CD4+ target cells. The virus protein p27 was detected by intracellular staining. Two different effector to target ratios were used, 0.5:1 and 2:1. The cells that were successfully infected with SHIV as indicated by p27 positivity was between 1.3%˜1.6%. In all the samples treated with CAR-T cells, the p27 positivity dropped to equal or less than 0.207%, which is near or lower than the flow staining isotype control. FIG. 19B shows the decreases of virus RNA and integrated DNA. In the cell culture supernatant, the virus titer dropped dramatically in the samples treated with CAR-T cells compared to the samples treated with UNT cells. The integrated DNA level dropped to almost undetectable level that there were some background noises detected due to the PCR process. All these data showed that our CAR-T were very efficient in clearing the SHIV infection in vitro.

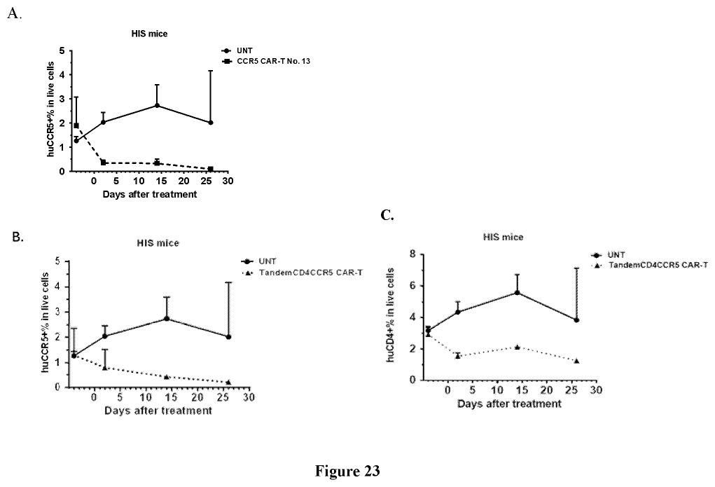

To test the in vivo efficacy of CAR constructs described herein, mice with human immune systems were used. The mice hereby will be referred as HIS mice. HIS mice were generated from the NOD CRISPR Prkdc IL2Rγ mice (NCG mice), which are severely immunocompromised and are deficient in T and B cell formation. Neonatal NCG mice were transplanted with human hematopoietic stem cells and were renamed as HIS mice. The human hematopoietic cells develop into T and B cells in the mice and fill the immunological niche in the mice. These HIS mice served as in vivo testing model for the CAR-T cells. Two CAR-T cells were tested in the in vivo model, CCR5 CAR-T (anti-CCR5 CAR) (FIG. 23A) and tandem CD4CCR5 CAR-T (anti-CD4-anti-CCR5 CAR) (FIGS. 23B and 23C). UNT cells were injected into a separate group of recipient mice as control. As indicated in FIG. 23A, in the UNT cell treated mice, the CCR5+ cells increased after the mice were inoculated with the UNT cells, while in the anti-CCR5 CAR-T treated mice, the CCR5+% continuously decreased during the observation period. At the end of the observation period, the CCR5+% in the live cell population was close to 0. Another group of HIS mice were treated with tandem CD4CCR5 CAR-T as shown in FIG. 23B and FIG. 23C. The CCR5+ population decreased dramatically in the mice peripheral blood after the CAR-T cell treatment. The CCR5+ population was almost completely depleted from the peripheral live cell population. The CD4+ population also decreased to half of the original population in the tandemCD4CCR5 CAR-T group. The percentage of CD4+ population in the live cells was ˜3% before the CAR-T treatment, and decreased to 1% at the end of the observation period. These data indicated that anti-CCR5 CAR-T was very efficient in vivo in eradicating the CCR5+ cells from peripheral blood. The CD4+ population was reduced to half and was still declining, suggesting that besides a great efficiency on eliminating CCR5+ cells, tandemCD4CCR5 CAR-T was also effective in reducing the CD4+ population.

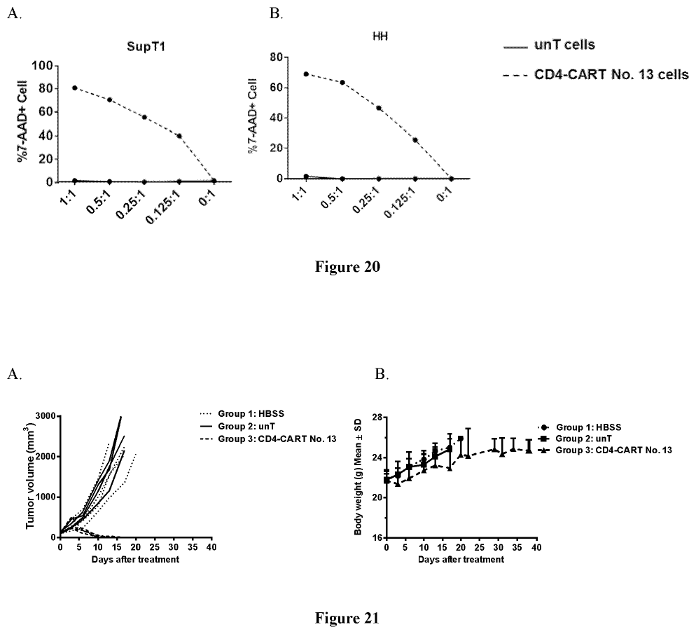

Another application of the anti-CD4 CAR-T cells could be for CD4+ T cell lymphoma/leukemia therapy. As shown in FIG. 20, different dosages of CAR-T cells were co-cultured with Sup-T1 and HH T lymphoblast cells, which were CD4+. CAR-T cells showed great cytotoxicity against these tumor cells while UNT control cells had no effect on the tumor cell viability. HH cells were implanted into NCG mice subcutaneously to generate a cell-derived xenograft (CDX) mouse model. In vivo inoculation of CAR-T cells into HH CDX mice showed that CAR-T cells can significantly reduce the mice tumor burden as shown in FIG. 21. The mice received CAR-T therapy were tumor-free at day 12 post CAR-T inoculation. As shown in FIG. 21A, the tumor grew continuously in control Hank's Balanced Salt Solution (HBSS) buffer or UNT cell treated mice before they met sacrificing criteria. The tumor in the CAR-T treated mice shrank quickly after the treatment and the mice survived the whole observation period. In FIG. 21B, the CAR-T treated mice weighs slightly lower than the HBSS or UNT treated mice, probably due to less tumor burden. The HBSS and UNT treated mice were sacrificed due to disease progress at around day 20, so there was no body weight record after that.

### Example 8. Split CAR Systems

Construction of Split CAR-T Cells

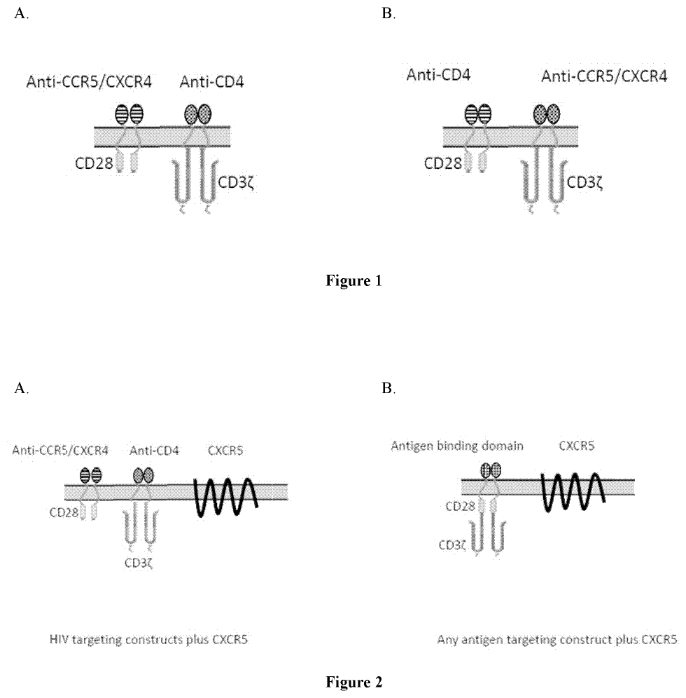

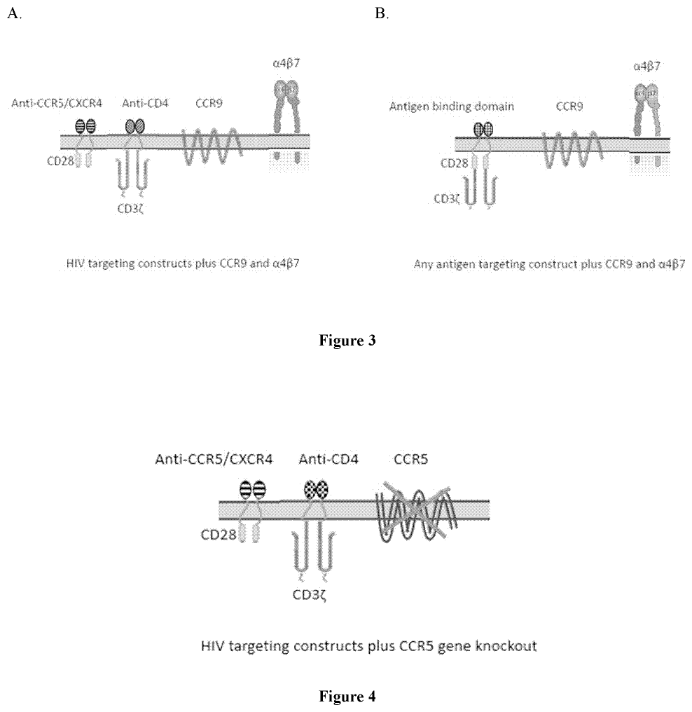

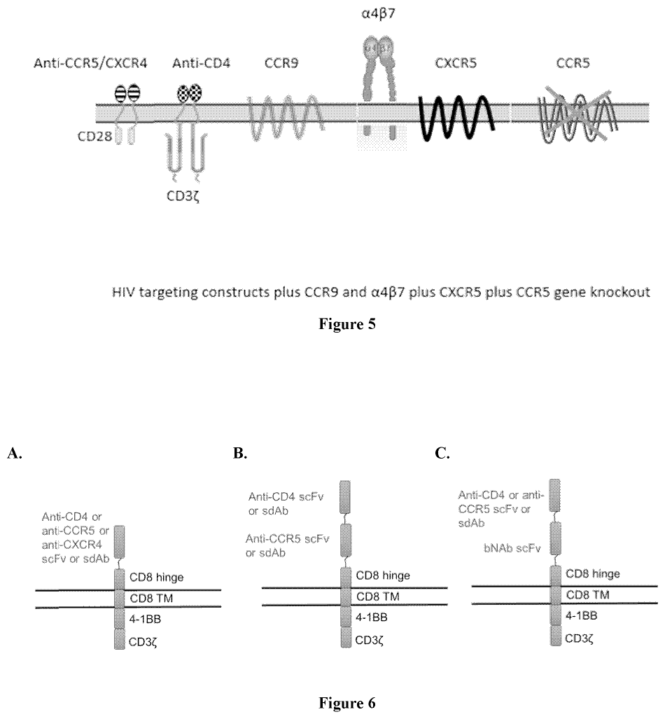

The split CARs system described herein contains two components. One protein is composed of an extracellular antigen-recognition domain, a hinge, a CD8 transmembrane domain and a CD28 intracellular costimulatory domain. The other protein of the split signal CARs is composed of a second antigen-recognition domain, a hinge, a CD8 transmembrane domain and an intracellular CD3zeta signal transduction domain. The extracellular antigen recognition domain could be an anti-CD4 scFv or sdAb for one component, and anti-CCR5 or anti-CXCR4 scFv or sdAb for the other component (FIG. 1). In the present example, the coding sequences for the two proteins were linked with a P2A sequence in-between. The whole coding sequence was under the control of EF1α promoter in a pLVX lenti-viral plasmid. The T cells successfully transduced with the CAR constructs will express both proteins synergistically. The T cells could also express homing receptors. One of the homing receptors could be CXCR5 (FIG. 2), which interacts with CXCL13 chemokine and plays important role for cell homing to B cell follicles in secondary lymphoid organs. The homing receptor could also be an upregulated α4β7, which helps cells home to the intestines (FIG. 3).

In Vitro Effect of Split Signal CAR

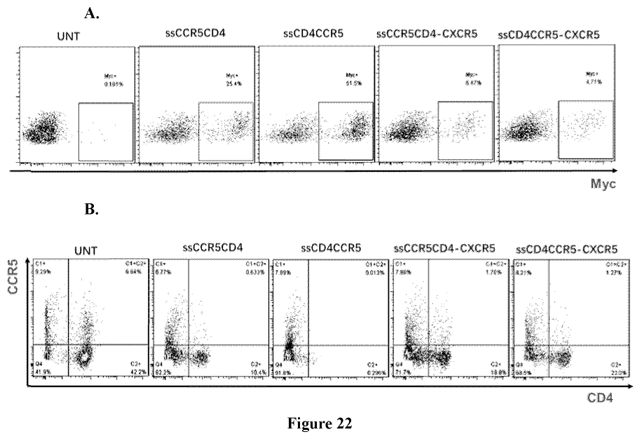

FIG. 22 shows the in vitro effect of exemplary split signal CAR-Ts. Four designs were used in this experiment. In ssCD4CCR5 CAR-T, anti-CD4 is connected with CD3ζ signaling domain and anti-CCR5 is connected with the co-stimulatory domain while in ssCCR5CD4, anti-CCR5 part contains CD3zeta signaling domain and anti-CD4 is connected to the co-stimulatory domain.

The constructs has the following sequences:

ssCD4CCR5 CAR-T: SEQ ID NO. 26

anti-CD4-CD8 hinge-CD8 TM-CD3ζ: SEQ ID NO. 27

anti-CCR5-CD8 hinge-CD8 TM-4-1-BB: SEQ ID NO. 28

ssCCR5CD4 CAR-T: SEQ ID NO. 29

anti-CCR5-CD8 hinge-CD8 TM-CD3ζ: SEQ ID NO. 30

anti-CD4-CD8 hinge-CD8 TM-4-1-BB: SEQ ID NO. 31

ssCD4CCR5-CXCR5: SEQ ID NO. 78

ssCCR5CD4-CXCR5: SEQ ID NO. 79

The two moieties were linked by a P2A in between, and the first moiety had a Myc tag. The Myc expression was detected by flow cytometry as shown in FIG. 22A to stand for the expression of split signal CAR. To test their functions, CFSE labeled pan T cells were used as target cells and co-incubated with the CAR-T cells at E:T=0.5:1 The flow cytometry result is shown in FIG. 23B. In the control UNT sample, CD4+ population was 42.2%, CCR5+ population was 9.29%, and CD4+CCR5+ population was 6.64%. SsCD4CCR5 CAR-T and ssCCR5CD4 CAR-T killed most of the CD4+CCR5+ double positive population that the remained CD4+CCR5+ population was less than 1%. In ssCCR5CD4 CAR-T samples, there was 10.4% of CD4 single positive and 6.77% CCR5 single positive cells left. In ssCD4CCR5 CAR-T samples the remaining CCR5+ cells were 7.89%. With the addition of CXCR5 to the ssCCR5CD4 CAR and ssCD4CCR5 CAR, most of the CCR5+ cells were kept, and about half of the CD4+ single positive cells remained alive after co-culturing with the CAR-T cells. These data suggest that the split CAR works the best when both CD4 and CCR5 existed on the same cell. SsCCR5CD4, ssCCR5CD4-CXCR5 and ssCD4CCR5-CXCR5 CAR-T cells were not efficient in killing CD4 single positive or CCR5 single positive cells that they could spare many of those single positive cells. CCR5-tropic HIV infection requires both CD4 and CCR5 expression on the same cell. Split CAR could eliminate the HIV target cells but spare some of the CD4 or CCR5 single positive cells, which are less susceptible to HIV infection.

Sequences of exemplary constructs according to embodiments of the invention:

| Seq ID | | |
|---|---|---|
| NO. | Name | Sequence |
| 1 | Human CD4 | MNRGVPFRHLLLVLQLALLPAATQGKKVVLGKKGDTVELTCTASQKKSIQ |
| | | FHWKNSNQIKILGNQGSFLTKGPSKLNDRADSRRSLWDQGNFPLIIKNLKIE |
| | | DSDTYICEVEDQKEEVQLLVFGLTANSDTHLLQGQSLTLTLESPPGSSPSVQ |
| | | CRSPRGKNIQGGKTLSVSQLELQDSGTWTCTVLQNQKKVEFKIDIVVLAFQ |
| | | KASSIVYKKEGEQVEFSFPLAFTVEKLTGSGELWWQAERASSSKSWITFDL |
| | | KNKEVSVKRVTQDPKLQMGKKLPLHLTLPQALPQYAGSGNLTLALEAKTG |
| | | KLHQEVNLVVMRATQLQKNLTCEVWGPTSPKLMLSLKLENKEAKVSKRE |
| | | KAVWVLNPEAGMWQCLLSDSGQVLLESNIKVLPTWSTPVQPMALIVLGGV |
| | | AGLLLFIGLGIFFCVRCRHRRRQAERMSQIKRLLSEKKTCQCPHRFQKTCSPI |
| 2 | CD8 hinge | TTTPAPRPPTPAPTIASQPLSLRPEACRPAAGGAVHTRGLDFACD |
| 3 | CD8 | IYIWAPLAGTCGVLLLSLVITLYC |
| | transmembrane | |
| | domain | |
| 4 | human 4-1BB | KRGRKKLLYIFKQPFMRPVQTTQEEDGCSCRFPEEEEGGCEL |
| | intracellular region | |
| 5 | CD3ζ signaling | RVKFSRSADAPAYKQGQNQLYNELNLGRREEYDVLDKRRGRDPEMGGKP |
| | transduction | RRKNPQEGLYNELQKDKMAEAYSEIGMKGERRRGKGHDGLYQGLSTATK |
| | domain | DTYDALHMQALPPR |
| 6 | T-cell surface | MQSGTHWRVLGLCLLSVGVWGQDGNEEMGGITQTPYKVSISGTTVILTCP |
| | glycoprotein CD3ε | QYPGSEILWQHNDKNIGGDEDDKNIGSDEDHLSLKEFSELEQSGYYVCYPR |
| | chain | GSKPEDANFYLYLRARVCENCMEMDVMSVATIVIVDICITGGLLLLVYYW |
| | | SKNRKAKAKPVTRGAGAGGRQRGQNKERPPPVPNPDYEPIRKGQRDLYSG |
| | | LNQRRI |
| 7 | CD3ε signal | MQSGTHWRVLGLCLLSVGVWGQ |
| | peptide | |
| 8 | CD3ε sequence | DGNEEMGGITQTPYKVSISGTTVILTCPQYPGSEILWQHNDKNIGGDEDDK |
| | without signal | NIGSDEDHLSLKEFSELEQSGYYVCYPRGSKPEDANFYLYLRARVCENCME |
| | peptide | MDVMSVATIVIVDICITGGLLLLVYYWSKNRKAKAKPVTRGAGAGGRQRG |
| | | QNKERPPPVPNPDYEPIRKGQRDLYSGLNQRRI |
| 9 | human CXCR5 | MNYPLTLEMDLENLEDLFWELDRLDNYNDTSLVENHLCPATEGPLMASFK |
| | | AVFVPVAYSLIFLLGVIGNVLVLVILERHRQTRSSTETFLFHLAVADLLLVFI |
| | | LPFAVAEGSVGWVLGTFLCKTVIALHKVNFYCSSLLLACIAVDRYLAIVHA |
| | | VHAYRHRRLLSIHITCGTIWLVGFLLALPEILFAKVSQGHHNNSLPRCTFSQ |
| | | ENQAETHAWFTSRFLYHVAGFLLPMLVMGWCYVGVVHRLRQAQRRPQR |
| | | QKAVRVAILVTSIFFLCWSPYHIVIFLDTLARLKAVDNTCKLNGSLPVAITM |
| | | CEFLGLAHCCLNPMLYTFAGVKFRSDLSRLLTKLGCTGPASLCQLFPSWRR |
| | | SSLSESENATSLTTF |
| 10 | VRC01 scFv | QVQLVQSGGQMKKPGESMRISCRASGYEFIDCTLNWIRLAPGKRPEWMG |
| | | WLKPRGGAVNYARPLQGRVTMTRDVYSDTAFLELRSLTVDDTAVYFCTR |
| | | GKNCDYNWDFEHWGRGTPVIVSSGGGGSGGGGSGGGGSEIVLTQSPGTLS |
| | | LSPGETAIISCRTSQYGSLAWYQQRPGQAPRLVIYSGSTRAAGIPDRFSGSR |
| | | WGPDYNLTISNLESGDFGVYYCQQYEFFGQGTKVQVDIK |
| 11 | anti-CD4 CAR | MALPVTALLLPLALLLHAARPQVQLQQWGAGLLKPSETLSLTCAVYGGSF |
| | | SGYYWSWIRQPPGKGLEWIGEINHSGSTNYNPSLKSRVTISVDTSKNQFSLK |
| | | LSSVTAADTAVYYCARVINWFDPWGQGTLVTGGGGSGGGGSGGGGSDIQ |
| | | MTQSPSSVSASVGDRVTITCRASQDISSWLAWYQHKPGKAPKLLIYAASSL |
| | | QSGVPSRFSGSGSGTDFTLTISSLQPEDFATYYCQQANSFPYTFGQGTKLEIK |
| | | TTTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEEE |
| | | EGGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPR |
| 12 | anti-CCR5 CAR | MALPVTALLLPLALLLHAARPQVQLVESGGGVVQPGRSLRLSCAASGFTLS |
| | | GYGMHWVRQAPGKGLEWVSLISYDGSNKYYADSVKGRFTISRDDSKNTL |
| | | YLRMNSLRAEDTAVYYCARGRNDFWSGYYTAGMDVWGQGTTVTVSSGG |
| | | GGSGGGGSGGGGSDIQMTQSPSSLSASVGDRVTITCQASQGIRKYLNWYQ |
| | | QKPGKVPKLLIYDASNLETGVPSRFSGSGSGTDFTFAISSLQPEDTATYYCQ |
| | | QYDDFPFTFGQGTRLEIKRTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYSE |
| | | IGMKGERRRGKGHDGLYQGLSTATKDTYDALHMQALPPR |
| 13 | tandem antiCD4- | MALPVTALLLPLALLLHAARPQVQLQQWGAGLLKPSETLSLTCAVYGGSF |
| | antiCCR5 CAR | SGYYWSWIRQPPGKGLEWIGEINHSGSTNYNPSLKSRVTISVDTSKNQFSLK |
| | | LSSVTAADTAVYYCARVINWFDPWGQGTLVTGGGGSGGGGSGGGGSDIQ |
| | | MTQSPSSVSASVGDRVTITCRASQDISSWLAWYQHKPGKAPKLLIYAASSL |
| | | QSGVPSRFSGSGSGTDFTLTISSLQPEDFATYYCQQANSFPYTFGQGTKLEIK |
| | | GGGGSGGGGSGGGGSQVQLVESGGGVVQPGRSLRLSCAASGFTLSGYGM |
| | | HWVRQAPGKGLEWVSLISYDGSNKYYADSVKGRFTISRDDSKNTLYLRMN |
| | | SLRAEDTAVYYCARGRNDFWSGYYTAGMDVWGQGTTVTVSSGGGGSGG |
| | | GGSGGGGSDIQMTQSPSSLSASVGDRVTITCQASQGIRKYLNWYQQKPGK |
| | | VPKLLIYDASNLETGVPSRFSGSGSGTDFTFAISSLQPEDTATYYCQQYDDF |
| | | PFTFGQGTRLEIKRTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGE |
| | | RRRGKGHDGLYQGLSTATKDTYDALHMQALPPR |
| 14 | anti-CD4 CAR | MALPVTALLLPLALLLHAARPQVQLQQWGAGLLKPSETLSLTCAVYGGSF |
| | with CXCR5 | SGYYWSWIRQPPGKGLEWIGEINHSGSTNYNPSLKSRVTISVDTSKNQFSLK |
| | | LSSVTAADTAVYYCARVINWFDPWGQGTLVTGGGGSGGGGSGGGGSDIQ |
| | | MTQSPSSVSASVGDRVTITCRASQDISSWLAWYQHKPGKAPKLLIYAASSL |
| | | QSGVPSRFSGSGSGTDFTLTISSLQPEDFATYYCQQANSFPYTFGQGTKLEIK |
| | | TTTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEEE |
| | | EGGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPRGSGATNFSLLKQAGDVEENPGPMNYPLTLE |
| | | MDLENLEDLFWELDRLDNYNDTSLVENHLCPATEGPLMASFKAVFVPVAY |
| | | SLIFLLGVIGNVLVLVILERHRQTRSSTETFLFHLAVADLLLVFILPFAVAEGS |
| | | VGWVLGTFLCKTVIALHKVNFYCSSLLLACIAVDRYLAIVHAVHAYRHRR |
| | | LLSIHITCGTIWLVGFLLALPEILFAKVSQGHHNNSLPRCTFSQENQAETHA |
| | | WFTSRFLYHVAGFLLPMLVMGWCYVGVVHRLRQAQRRPQRQKAVRVAIL |
| | | VTSIFFLCWSPYHIVIFLDTLARLKAVDNTCKLNGSLPVAITMCEFLGLAHC |
| | | CLNPMLYTFAGVKFRSDLSRLLTKLGCTGPASLCQLFPSWRRSSLSESENAT |
| | | SLTTF |
| 15 | anti-CCR5 CAR | MALPVTALLLPLALLLHAARPQVQLVESGGGVVQPGRSLRLSCAASGFTLS |
| | with CXCR5 | GYGMHWVRQAPGKGLEWVSLISYDGSNKYYADSVKGRFTISRDDSKNTL |
| | | YLRMNSLRAEDTAVYYCARGRNDFWSGYYTAGMDVWGQGTTVTVSSGG |
| | | GGSGGGGSGGGGSDIQMTQSPSSLSASVGDRVTITCQASQGIRKYLNWYQ |
| | | QKPGKVPKLLIYDASNLETGVPSRFSGSGSGTDFTFAISSLQPEDTATYYCQ |
| | | QYDDFPFTFGQGTRLEIKRTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYSE |
| | | IGMKGERRRGKGHDGLYQGLSTATKDTYDALHMQALPPRGSGATNFSLLK |
| | | QAGDVEENPGPMNYPLTLEMDLENLEDLFWELDRLDNYNDTSLVENHLCP |
| | | ATEGPLMASFKAVFVPVAYSLIFLLGVIGNVLVLVILERHRQTRSSTETFLFH |
| | | LAVADLLLVFILPFAVAEGSVGWVLGTFLCKTVIALHKVNFYCSSLLLACIA |
| | | VDRYLAIVHAVHAYRHRRLLSIHITCGTIWLVGFLLALPEILFAKVSQGHHN |
| | | NSLPRCTFSQENQAETHAWFTSRFLYHVAGFLLPMLVMGWCYVGVVHRL |
| | | RQAQRRPQRQKAVRVAILVTSIFFLCWSPYHIVIFLDTLARLKAVDNTCKLN |
| | | GSLPVAITMCEFLGLAHCCLNPMLYTFAGVKFRSDLSRLLTKLGCTGPASL |
| | | CQLFPSWRRSSLSESENATSLTTF |
| 16 | tandem antiCD4- | MALPVTALLLPLALLLHAARPQVQLQQWGAGLLKPSETLSLTCAVYGGSF |
| | antiCCR5 CAR | SGYYWSWIRQPPGKGLEWIGEINHSGSTNYNPSLKSRVTISVDTSKNQFSLK |
| | with CXCR5 | LSSVTAADTAVYYCARVINWFDPWGQGTLVTGGGGSGGGGSGGGGSDIQ |
| | | MTQSPSSVSASVGDRVTITCRASQDISSWLAWYQHKPGKAPKLLIYAASSL |
| | | QSGVPSRFSGSGSGTDFTLTISSLQPEDFATYYCQQANSFPYTFGQGTKLEIK |
| | | GGGGSGGGGSGGGGSQVQLVESGGGVVQPGRSLRLSCAASGFTLSGYGM |
| | | HWVRQAPGKGLEWVSLISYDGSNKYYADSVKGRFTISRDDSKNTLYLRMN |
| | | SLRAEDTAVYYCARGRNDFWSGYYTAGMDVWGQGTTVTVSSGGGGSGG |
| | | GGSGGGGSDIQMTQSPSSLSASVGDRVTITCQASQGIRKYLNWYQQKPGK |
| | | VPKLLIYDASNLETGVPSRFSGSGSGTDFTFAISSLQPEDTATYYCQQYDDF |
| | | PFTFGQGTRLEIKRTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGE |
| | | RRRGKGHDGLYQGLSTATKDTYDALHMQALPPRGSGATNFSLLKQAGDV |
| | | EENPGPMNYPLTLEMDLENLEDLFWELDRLDNYNDTSLVENHLCPATEGP |
| | | LMASFKAVFVPVAYSLIFLLGVIGNVLVLVILERHRQTRSSTETFLFHLAVA |
| | | DLLLVFILPFAVAEGSVGWVLGTFLCKTVIALHKVNFYCSSLLLACIAVDRY |
| | | LAIVHAVHAYRHRRLLSIHITCGTIWLVGFLLALPEILFAKVSQGHHNNSLP |
| | | RCTFSQENQAETHAWFTSRFLYHVAGFLLPMLVMGWCYVGVVHRLRQAQ |
| | | RRPQRQKAVRVAILVTSIFFLCWSPYHIVIFLDTLARLKAVDNTCKLNGSLP |
| | | VAITMCEFLGLAHCCLNPMLYTFAGVKFRSDLSRLLTKLGCTGPASLCQLF |
| | | PGWRRSSLSESENATSLTTF |
| 17 | anti-CD4 | CARMALPVTALLLPLALLLHAARPQVQLQQWGAGLLKPSETLSLTCAVYGGSF |
| | with CXCR5 and | SGYYWSWIRQPPGKGLEWIGEINHSGSTNYNPSLKSRVTISVDTSKNQFSLK |
| | VRC01 | LSSVTAADTAVYYCARVINWFDPWGQGTLVTGGGGSGGGGSGGGGSDIQ |
| | | MTQSPSSVSASVGDRVTITCRASQDISSWLAWYQHKPGKAPKLLIYAASSL |
| | | QSGVPSRFSGSGSGTDFTLTISSLQPEDFATYYCQQANSFPYTFGQGTKLEIK |
| | | TTTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEEE |
| | | EGGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPRGSGATNFSLLKQAGDVEENPGPMNYPLTLE |
| | | MDLENLEDLFWELDRLDNYNDTSLVENHLCPATEGPLMASFKAVFVPVAY |
| | | SLIFLLGVIGNVLVLVILERHRQTRSSTETFLFHLAVADLLLVFILPFAVAEGS |
| | | VGWVLGTFLCKTVIALHKVNFYCSSLLLACIAVDRYLAIVHAVHAYRHRR |
| | | LLSIHITCGTIWLVGFLLALPEILFAKVSQGHHNNSLPRCTFSQENQAETHA |
| | | WFTSRFLYHVAGFLLPMLVMGWCYVGVVHRLRQAQRRPQRQKAVRVAIL |
| | | VTSIFFLCWSPYHIVIFLDTLARLKAVDNTCKLNGSLPVAITMCEFLGLAHC |
| | | CLNPMLYTFAGVKFRSDLSRLLTKLGCTGPASLCQLFPSWRRSSLSESENAT |
| | | SLTTFGSGATNFSLLKQAGDVEENPGPMYRNIQLLSCIALSLALVTNSQVQL |
| | | VQSGGQMKKPGESMRISCRASGYEFIDCTLNWIRLAPGKRPEWMGWLKPR |
| | | GGAVNYARPLQGRVTMTRDVYSDTAFLELRSLTVDDTAVYFCTRGKNCD |
| | | YNWDFEHWGRGTPVIVSSGGGGSGGGGSGGGGSEIVLTQSPGTLSLSPGET |
| | | AIISCRTSQYGSLAWYQQRPGQAPRLVIYSGSTRAAGIPDRFSGSRWGPDYN |
| | | LTISNLESGDFGVYYCQQYEFFGQGTKVQVDIKHHHHHH |
| 18 | anti-CCR5 CAR | MALPVTALLLPLALLLHAARPQVQLVESGGGVVQPGRSLRLSCAASGFTLS |
| | with CXCR5 and | GYGMHWVRQAPGKGLEWVSLISYDGSNKYYADSVKGRFTISRDDSKNTL |
| | VRC01 | YLRMNSLRAEDTAVYYCARGRNDFWSGYYTAGMDVWGQGTTVTVSSGG |
| | | GGSGGGGSGGGGSDIQMTQSPSSLSASVGDRVTITCQASQGIRKYLNWYQ |
| | | QKPGKVPKLLIYDASNLETGVPSRFSGSGSGTDFTFAISSLQPEDTATYYCQ |
| | | QYDDFPFTFGQGTRLEIKRTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYSE |
| | | IGMKGERRRGKGHDGLYQGLSTATKDTYDALHMQALPPRGSGATNFSLLK |
| | | QAGDVEENPGPMNYPLTLEMDLENLEDLFWELDRLDNYNDTSLVENHLCP |
| | | ATEGPLMASFKAVFVPVAYSLIFLLGVIGNVLVLVILERHRQTRSSTETFLFH |
| | | LAVADLLLVFILPFAVAEGSVGWVLGTFLCKTVIALHKVNFYCSSLLLACIA |
| | | VDRYLAIVHAVHAYRHRRLLSIHITCGTIWLVGFLLALPEILFAKVSQGHHN |
| | | NSLPRCTFSQENQAETHAWFTSRFLYHVAGFLLPMLVMGWCYVGVVHRL |
| | | RQAQRRPQRQKAVRVAILVTSIFFLCWSPYHIVIFLDTLARLKAVDNTCKLN |
| | | GSLPVAITMCEFLGLAHCCLNPMLYTFAGVKFRSDLSRLLTKLGCTGPASL |
| | | CQLFPSWRRSSLSESENATSLTTFGSGATNFSLLKQAGDVEENPGPMYRMQ |
| | | LLSCIALSLALVTNSQVQLVQSGGQMKKPGESMRISCRASGYEFIDCTLNWI |
| | | RLAPGKRPEWMGWLKPRGGAVNYARPLQGRVTMTRDVYSDTAFLELRSL |
| | | TVDDTAVYFCTRGKNCDYNWDFEHWGRGTPVIVSSGGGGSGGGGSGGGG |
| | | SEIVLTQSPGTLSLSPGETAIISCRTSQYGSLAWYQQRPGQAPRLVIYSGSTR |
| | | AAGIPDRFSGSRWGPDYNLTISNLESGDFGVYYCQQYEFFGQGTKVQVDIK |
| | | HHHHHH |
| 19 | tandem antiCD4- | MALPVTALLLPLALLLHAARPQVQLQQWGAGLLKPSETLSLTCAVYGGSF |
| | antiCCR5 CAR | SGYYWSWIRQPPGKGLEWIGEINHSGSTNYNPSLKSRVTISVDTSKNQFSLK |
| | with CXCR5 and | LSSVTAADTAVYYCARVINWFDPWGQGTLVTGGGGSGGGGSGGGGSDIQ |
| | VRC01 | MTQSPSSVSASVGDRVTITCRASQDISSWLAWYQHKPGKAPKLLIYAASSL |
| | | QSGVPSRFSGSGSGTDFTLTISSLQPEDFATYYCQQANSFPYTFGQGTKLEIK |
| | | GGGGSGGGGSGGGGSQVQLVESGGGVVQPGRSLRLSCAASGFTLSGYGM |
| | | HWVRQAPGKGLEWVSLISYDGSNKYYADSVKGRFTISRDDSKNTLYLRWIN |
| | | SLRAEDTAVYYCARGRNDFWSGYYTAGMDVWGQGTTVTVSSGGGGSGG |
| | | GGSGGGGSDIQMTQSPSSLSASVGDRVTITCQASQGIRKYLNWYQQKPGK |
| | | VPKWYDASNLETGVPSRFSGSGSGTDFTFAISSLQPEDTATYYCQQYDDF |
| | | PFTFGQGTRLEIKRTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGE |
| | | RRRGKGHDGLYQGLSTATKDTYDALHMQALPPRGSGATNFSLLKQAGDV |
| | | EENPGPMNYPLTLEMDLENLEDLFWELDRLDNYNDTSLVENHLCPATEGP |
| | | LMASFKAVFVPVAYSLIFLLGVIGNVLVLVILERHRQTRSSTETFLFHLAVA |
| | | DLLLVFILPFAVAEGSVGWVLGTFLCKTVIALHKVNFYCSSLLLACIAVDRY |
| | | LAIVHAVHAYRHRRLLSIHITCGTIWLVGFLLALPEILFAKVSQGHHNNSLP |
| | | RCTFSQENQAETHAWFTSRFLYHVAGFLLPMLVMGWCYVGVVHRLRQAQ |
| | | RRPQRQKAVRVAILVTSIFFLCWSPYHIVIFLDTLARLKAVDNTCKLNGSLP |
| | | VAITMCEFLGLAHCCLNPMLYTFAGVKFRSDLSRLLTKLGCTGPASLCQLF |
| | | PGWRRSSLSESENATSLTTFGSGATNFSLLKQAGDVEENPGPMYRMQLLSC |
| | | IALSLALVTNSQVQLVQSGGQMKKPGESMRISCRASGYEFIDCTLNWIRLA |
| | | PGKRPEWMGWLKPRGGAVNYARPLQGRVTMTRDVYSDTAFLELRSLTVD |
| | | DTAVYFCTRGKNCDYNWDFEHWGRGTPVIVSSGGGGSGGGGSGGGGSEI |
| | | VLTQSPGTLSLSPGETAIISCRTSQYGSLAWYQQRPGQAPRLVIYSGSTRAA |
| | | GIPDRFSGSRWGPDYNLTISNLESGDFGVYYCQQYEFFGQGTKVQVDIKHH |
| | | HHHH |
| 20 | anti-CD4 eTCR | MQSGTHWRVLGLCLLSVGVWGQQVQLQQWGAGLLKPSETLSLTCAVYG |
| | | GSFSGYYWSWIRQPPGKGLEWIGEINHSGSTNYNPSLKSRVTISVDTSKNQF |
| | | SLKLSSVTAADTAVYYCARVINWFDPWGQGTLVTGGGGSGGGGSGGGGS |
| | | DIQMTQSPSSVSASVGDRVTITCRASQDISSWLAWYQHKPGKAPKLLIYAA |
| | | SSLQSGVPSRFSGSGSGTDFTLTISSLQPEDFATYYCQQANSFPYTFGQGTKL |
| | | EIKGGGGSGGGGSGGGGSDGNEEMGGITQTPYKVSISGTTVILTCPQYPGSE |
| | | ILWQHNDKNIGGDEDDKNIGSDEDHLSLKEFSELEQSGYYVCYPRGSKPED |
| | | ANFYLYLRARVCENCMEMDVMSVATIVIVDICITGGLLLLVYYWSKNRKA |
| | | KAKPVTRGAGAGGRQRGQNKERPPPVPNPDYEPIRKGQRDLYSGLNQRRI |
| 21 | anti-CCR5 eTCR | MQSGTHWRVLGLCLLSVGVWGQEVQLVESGGGLVQPKGSLKLSCAASGF |
| | | TFNTYAMNWVRQAPGKGLEWVARIRNKSNNYATYYAASVKDRFTISRDD |
| | | SQSMLYLQMNNLKTEDTAMYYCVSLGEFAYWGQGTLVTVSAGGGGSGG |
| | | GGSGGGGSEIVLTQSPTTMAASPGEKVTITCSATSTINSNYLHWYQQKPGFS |
| | | PKLLIYRTSNLASGVPARFSGSGSGTSYSLTIGTMEAEDVATYYCQQGSTLP |
| | | FTFGSGTKLEIKGGGGSGGGGSGGGGSDGNEEMGGITQTPYKVSISGTTVIL |
| | | TCPQYPGSEILWQHNDKNIGGDEDDKNIGSDEDHLSLKEFSELEQSGYYVC |
| | | YPRGSKPEDANFYLYLRARVCENCMEMDVMSVATIVIVDICITGGLLLLVY |
| | | YWSKNRKAKAKPVTRGAGAGGRQRGQNKERPPPVPNPDYEPIRKGQRDL |
| | | YSGLNQRRI |
| 22 | anti-CD4 eTCR | MQSGTHWRVLGLCLLSVGVWGQQVQLQQWGAGLLKPSETLSLTCAVYG |
| | with CXCR5 | GSFSGYYWSWIRQPPGKGLEWIGEINHSGSTNYNPSLKSRVTISVDTSKNQF |
| | | SLKLSSVTAADTAVYYCARVINWFDPWGQGTLVTGGGGSGGGGSGGGGS |
| | | DIQMTQSPSSVSASVGDRVTITCRASQDISSWLAWYQHKPGKAPKLLIYAA |
| | | SSLQSGVPSRFSGSGSGTDFTLTISSLQPEDFATYYCQQANSFPYTFGQGTKL |
| | | EIKGGGGSGGGGSGGGGSDGNEEMGGITQTPYKVSISGTTVILTCPQYPGSE |
| | | ILWQHNDKNIGGDEDDKNIGSDEDHLSLKEFSELEQSGYYVCYPRGSKPED |
| | | ANFYLYLRARVCENCMEMDVMSVATIVIVDICITGGLLLLVYYWSKNRKA |
| | | KAKPVTRGAGAGGRQRGQNKERPPPVPNPDYEPIRKGQRDLYSGLNQRRI |
| | | GSGATNFSLLKQAGDVEENPGPMNYPLTLEMDLENLEDLFWELDRLDNYN |
| | | DTSLVENHLCPATEGPLMASFKAVFVPVAYSLIFLLGVIGNVLVLVILERHR |
| | | QTRSSTETFLFHLAVADLLLVFILPFAVAEGSVGWVLGTFLCKTVIALHKVN |
| | | FYCSSLLLACIAVDRYLAIVHAVHAYRHRRLLSIHITCGTIWLVGFLLALPEI |
| | | LFAKVSQGHHNNSLPRCTFSQENQAETHAWFTSRFLYHVAGFLLPMLVMG |
| | | WCYVGVVHRLRQAQRRPQRQKAVRVAILVTSIFFLCWSPYHIVIFLDTLAR |
| | | LKAVDNTCKLNGSLPVAITMCEFLGLAHCCLNPMLYTFAGVKFRSDLSRLL |
| | | TKLGCTGPASLCQLFPGWRRSSLSESENATSLTTF |
| 23 | anti-CCR5 eTCR | MQSGTHWRVLGLCLLSVGVWGQQVQLVESGGGVVQPGRSLRLSCAASGF |
| | with CXCR5 | TLSGYGMHWVRQAPGKGLEWVSLISYDGSNKYYADSVKGRFTISRDDSKN |
| | | TLYLRMNSLRAEDTAVYYCARGRNDFWSGYYTAGMDVWGQGTTVTVSS |
| | | GGGGSGGGGSGGGGSDIQMTQSPSSLSASVGDRVTITCQASQGIRKYLNW |
| | | YQQKPGKVPKLLIYDASNLETGVPSRFSGSGSGTDFTFAISSLQPEDTATYY |
| | | CQQYDDFPFTFGQGTRLEIKRGGGGSGGGGSGGGGSDGNEEMGGITQTPY |
| | | KVSISGTTVILTCPQYPGSEILWQHNDKNIGGDEDDKNIGSDEDHLSLKEFS |
| | | ELEQSGYYVCYPRGSKPEDANFYLYLRARVCENCMEMDVMSVATIVIVDI |
| | | CITGGLLLLVYYWSKNRKAKAKPVTRGAGAGGRQRGQNKERPPPVPNPD |
| | | YEPIRKGQRDLYSGLNQRRIGSGATNFSLLKQAGDVEENPGPMNYPLTLEM |
| | | DLENLEDLFWELDRLDNYNDTSLVENHLCPATEGPLMASFKAVFVPVAYS |
| | | LIFLLGVIGNVLVLVILERHRQTRSSTETFLFHLAVADLLLVFILPFAVAEGS |
| | | VGWVLGTFLCKTVIALHKVNFYCSSLLLACIAVDRYLAIVHAVHAYRHRR |
| | | LLSIHITCGTIWLVGFLLALPEILFAKVSQGHHNNSLPRCTFSQENQAETHA |
| | | WFTSRFLYHVAGFLLPMLVMGWCYVGVVHRLRQAQRRPQRQKAVRVAIL |
| | | VTSIFFLCWSPYHIVIFLDTLARLKAVDNTCKLNGSLPVAITMCEFLGLAHC |
| | | CLNPMLYTFAGVKFRSDLSRLLTKLGCTGPASLCQLFPGWRRSSLSESENA |
| | | TSLTTF |
| 24 | tandem antiCD4- | MQSGTHWRVLGLCLLSVGVWGQQVQLQQWGAGLLKPSETLSLTCAVYG |
| | anti-CCR5 eTCR | GSFSGYYWSWIRQPPGKGLEWIGEINHSGSTNYNPSLKSRVTISVDTSKNQF |
| | with CXCR5 | SLKLSSVTAADTAVYYCARVINWFDPWGQGTLVTGGGGSGGGGSGGGGS |
| | | DIQMTQSPSSVSASVGDRVTITCRASQDISSWLAWYQHKPGKAPKLLIYAA |
| | | SSLQSGVPSRFSGSGSGTDFTLTISSLQPEDFATYYCQQANSFPYTFGQGTKL |
| | | EIKGGGGSGGGGSGGGGSQVQLVESGGGVVQPGRSLRLSCAASGFTLSGY |
| | | GMHWVRQAPGKGLEWVSLISYDGSNKYYADSVKGRFTISRDDSKNTLYLR |
| | | MNSLRAEDTAVYYCARGRNDFWSGYYTAGMDVWGQGTTVTVSSGGGGS |
| | | GGGGSGGGGSDIQMTQSPSSLSASVGDRVTITCQASQGIRKYLNWYQQKP |
| | | GKVPKLLIYDASNLETGVPSRFSGSGSGTDFTFAISSLQPEDTATYYCQQYD |
| | | DFPFTFGQGTRLEIKRGGGGSGGGGSGGGGSDGNEEMGGITQTPYKVSISG |
| | | TTVILTCPQYPGSEILWQHNDKNIGGDEDDKNIGSDEDHLSLKEFSELEQSG |
| | | YYVCYPRGSKPEDANFYLYLRARVCENCMEMDVMSVATIVIVDICITGGLL |
| | | LLVYYWSKNRKAKAKPVTRGAGAGGRQRGQNKERPPPVPNPDYEPIRKG |
| | | QRDLYSGLNQRRIGSGATNFSLLKQAGDVEENPGPMNYPLTLEMDLENLE |
| | | DLFWELDRLDNYNDTSLVENHLCPATEGPLMASFKAVFVPVAYSLIFLLGV |
| | | IGNVLVLVILERHRQTRSSTETFLFHLAVADLLLVFILPFAVAEGSVGWVLG |
| | | TFLCKTVIALHKVNFYCSSLLLACIAVDRYLAIVHAVHAYRHRRLLSIHITC |
| | | GTIWLVGFLLALPEILFAKVSQGHHNNSLPRCTFSQENQAETHAWFTSRFL |
| | | YHVAGFLLPMLVMGWCYVGVVHRLRQAQRRPQRQKAVRVAILVTSIFFL |
| | | CWSPYHIVIFLDTLARLKAVDNTCKLNGSLPVAITMCEFLGLAHCCLNPML |
| | | YTFAGVKFRSDLSRLLTKLGCTGPASLCQLFPSWRRSSLSESENATSLTTF |
| 25 | tandem antiCD4- | MQSGTHWRVLGLCLLSVGVWGQQVQLQQWGAGLLKPSETLSLTCAVYG |
| | anti-CCR5 eTCR | GSFSGYYWSWIRQPPGKGLEWIGEINHSGSTNYNPSLKSRVTISVDTSKNQF |
| | with CXCR5 and | SLKLSSVTAADTAVYYCARVINWFDPWGQGTLVTGGGGSGGGGSGGGGS |
| | VRC01 | DIQMTQSPSSVSASVGDRVTITCRASQDISSWLAWYQHKPGKAPKLLIYAA |
| | | SSLQSGVPSRFSGSGSGTDFTLTISSLQPEDFATYYCQQANSFPYTFGQGTKL |
| | | EIKGGGGSGGGGSGGGGSQVQLVESGGGVVQPGRSLRLSCAASGFTLSGY |
| | | GMHWVRQAPGKGLEWVSLISYDGSNKYYADSVKGRFTISRDDSKNTLYLR |
| | | MNSLRAEDTAVYYCARGRNDFWSGYYTAGMDVWGQGTTVTVSSGGGGS |
| | | GGGGSGGGGSDIQMTQSPSSLSASVGDRVTITCQASQGIRKYLNWYQQKP |
| | | GKVPKLLIYDASNLETGVPSRFSGSGSGTDFTFAISSLQPEDTATYYCQQYD |
| | | DFPFTFGQGTRLEIKRGGGGSGGGGSGGGGSDGNEEMGGITQTPYKVSISG |
| | | TTVILTCPQYPGSEILWQHNDKNIGGDEDDKNIGSDEDHLSLKEFSELEQSG |
| | | YYVCYPRGSKPEDANFYLYLRARVCENCMEMDVMSVATIVIVDICITGGLL |
| | | LLVYYWSKNRKAKAKPVTRGAGAGGRQRGQNKERPPPVPNPDYEPIRKG |
| | | QRDLYSGLNQRRIGSGATNFSLLKQAGDVEENPGPMNYPLTLEMDLENLE |
| | | DLFWELDRLDNYNDTSLVENHLCPATEGPLMASFKAVFVPVAYSLIFLLGV |
| | | IGNVLVLVILERHRQTRSSTETFLFHLAVADLLLVFILPFAVAEGSVGWVLG |
| | | TFLCKTVIALHKVNFYCSSLLLACIAVDRYLAIVHAVHAYRHRRLLSIHITC |
| | | GTIWLVGFLLALPEILFAKVSQGHHNNSLPRCTFSQENQAETHAWFTSRFL |
| | | YHVAGFLLPMLVMGWCYVGVVHRLRQAQRRPQRQKAVRVAILVTSIFFL |
| | | CWSPYHIVIFLDTLARLKAVDNTCKLNGSLPVAITMCEFLGLAHCCLNPML |
| | | YTFAGVKFRSDLSRLLTKLGCTGPASLCQLFPSWRRSSLSESENATSLTTFG |
| | | SGATNFSLLKQAGDVEENPGPMYRMQLLSCIALSLALVTNSQVQLVQSGG |
| | | QMKKPGESMRISCRASGYEFIDCTLNWIRLAPGKRPEWMGWLKPRGGAVN |
| | | YARPLQGRVTMTRDVYSDTAFLELRSLTVDDTAVYFCTRGKNCDYNWDF |
| | | EHWGRGTPVIVSSGGGGSGGGGSGGGGSEIVLTQSPGTLSLSPGETAIISCRT |
| | | SQYGSLAWYQQRPGQAPRLVIYSGSTRAAGIPDRFSGSRWGPDYNLTISNL |
| | | ESGDFGVYYCQQYEFFGQGTKVQVDIKHHHHHH |
| 26 | split signal | MALPVTALLLPLALLLHAARPEQKLISEEDLQVQLQQWGAGLLKPSETLSL |
| | antiCD4-antiCCR5 | TCAVYGGSFSGYYWSWIRQPPGKGLEWIGEINHSGSTNYNPSLKSRVTISV |
| | CAR | DTSKNQFSLKLSSVTAADTAVYYCARVINWFDPWGQGTLVTGGGGSGGG |
| | | GSGGGGSDIQMTQSPSSVSASVGDRVTITCRASQDISSWLAWYQHKPGKAP |
| | | KLLIYAASSLQSGVPSRFSGSGSGTDFTLTISSLQPEDFATYYCQQANSFPYT |
| | | FGQGTKLEIKTTTPAPRPPTPAPTIASQPLSLRPEACRPAAGGAVHTRGLDFA |
| | | CDIYIWAPLAGTCGVLLLSLVITLYCRVKFSRSADAPAYKQGQNQLYNELN |
| | | LGRREEYDVLDKRRGRDPEMGGKPRRKNPQEGLYNELQKDKMAEAYSEI |
| | | GMKGERRRGKGHDGLYQGLSTATKDTYDALHMQALPPRGSGATNFSLLK |
| | | QAGDVEENPGPMALPVTALLLPLALLLHAARPYPYDVPDYAQVQLVESGG |
| | | GVVQPGRSLRLSCAASGFTLSGYGMHWVRQAPGKGLEWVSLISYDGSNK |
| | | YYADSVKGRFTISRDDSKNTLYLRMNSLRAEDTAVYYCARGRNDFWSGY |
| | | YTAGMDVWGQGTTVTVSSGGGGSGGGGSGGGGSDIQMTQSPSSLSASVG |
| | | DRVTITCQASQGIRKYLNWYQQKPGKVPKLLIYDASNLETGVPSRFSGSGS |
| | | GTDFTFAISSLQPEDTATYYCQQYDDFPFTFGQGTRLEIKRTTTPAPRPPTPA |
| | | PTIASQPLSLRPEACRPAAGGAVHTRGLDFACDIYIWAPLAGTCGVLLLSLV |
| | | ITLYCRSKRSRLLHSDYMNMTPRRPGPTRKHYQPYAPPRDFAAYRSKRGR |
| | | KKLLYIFKQPFMRPVQTTQEEDGCSCRFPEEEEGGCEL |
| 27 | anti-CD4-CD8 | MALPVTALLLPLALLLHAARPEQKLISEEDLQVQLQQWGAGLLKPSETLSL |
| | hinge-CD8 TM- | TCAVYGGSFSGYYWSWIRQPPGKGLEWIGEINHSGSTNYNPSLKSRVTISV |
| | CD3ζ: | DTSKNQFSLKLSSVTAADTAVYYCARVINWFDPWGQGTLVTGGGGSGGG |
| | | GSGGGGSDIQMTQSPSSVSASVGDRVTITCRASQDISSWLAWYQHKPGKAP |
| | | KLLIYAASSLQSGVPSRFSGSGSGTDFTLTISSLQPEDFATYYCQQANSFPYT |
| | | FGQGTKLEIKTTTPAPRPPTPAPTIASQPLSLRPEACRPAAGGAVHTRGLDFA |
| | | CDIYIWAPLAGTCGVLLLSLVITLYCRVKFSRSADAPAYKQGQNQLYNELN |
| | | LGRREEYDVLDKRRGRDPEMGGKPRRKNPQEGLYNELQKDKMAEAYSEI |
| | | GMKGERRRGKGHDGLYQGLSTATKDTYDALHMQALPPR |
| 28 | anti-CCR5-CD8 | MALPVTALLLPLALLLHAARPYPYDVPDYAQVQLVESGGGVVQPGRSLRL |
| | hinge-CD8 TM-4- | SCAASGFTLSGYGMHWVRQAPGKGLEWVSLISYDGSNKYYADSVKGRFTI |
| | 1-BB | SRDDSKNTLYLRMNSLRAEDTAVYYCARGRNDFWSGYYTAGMDVWGQG |
| | | TTVTVSSGGGGSGGGGSGGGGSDIQMTQSPSSLSASVGDRVTITCQASQGIR |
| | | KYLNWYQQKPGKVPKLLIYDASNLETGVPSRFSGSGSGTDFTFAISSLQPED |
| | | TATYYCQQYDDFPFTFGQGTRLEIKRTTTPAPRPPTPAPTIASQPLSLRPEAC |
| | | RPAAGGAVHTRGLDFACDIYIWAPLAGTCGVLLLSLVITLYCRSKRSRLLH |
| | | SDYMNMTPRRPGPTRKHYQPYAPPRDFAAYRSKRGRKKLLYIFKQPFMRP |
| | | VQTTQEEDGCSCRFPEEEEGGCEL |
| 29 | split signal | MALPVTALLLPLALLLHAARPEQKLISEEDLQVQLVESGGGVVQPGRSLRL |
| | antiCCR5-antiCD4 | SCAASGFTLSGYGMHWVRQAPGKGLEWVSLISYDGSNKYYADSVKGRFTI |
| | CAR | SRDDSKNTLYLRMNSLRAEDTAVYYCARGRNDFWSGYYTAGMDVWGQG |
| | | TTVTVSSGGGGSGGGGSGGGGSDIQMTQSPSSLSASVGDRVTITCQASQGIR |
| | | KYLNWYQQKPGKVPKLLIYDASNLETGVPSRFSGSGSGTDFTFAISSLQPED |
| | | TATYYCQQYDDFPFTFGQGTRLEIKRTTTPAPRPPTPAPTIASQPLSLRPEAC |
| | | RPAAGGAVHTRGLDFACDIYIWAPLAGTCGVLLLSLVITLYCRVKFSRSAD |
| | | APAYKQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHM |
| | | QALPPRGSGATNFSLLKQAGDVEENPGPMALPVTALLLPLALLLHAARPYP |
| | | YDVPDYAQVQLQQWGAGLLKPSETLSLTCAVYGGSFSGYYWSWIRQPPG |
| | | KGLEWIGEINHSGSTNYNPSLKSRVTISVDTSKNQFSLKLSSVTAADTAVYY |
| | | CARVINWFDPWGQGTLVTGGGGSGGGGSGGGGSDIQMTQSPSSVSASVGD |
| | | RVTITCRASQDISSWLAWYQHKPGKAPKLLIYAASSLQSGVPSRFSGSGSGT |
| | | DFTLTISSLQPEDFATYYCQQANSFPYTFGQGTKLEIKTTTPAPRPPTPAPTIA |
| | | SQPLSLRPEACRPAAGGAVHTRGLDFACDIYIWAPLAGTCGVLLLSLVITLY |
| | | CRSKRSRLLHSDYMNMTPRRPGPTRKHYQPYAPPRDFAAYRSKRGRKKLL |
| | | YIFKQPFMRPVQTTQEEDGCSCRFPEEEEGGCEL |
| 30 | anti-CCR5-CD8 | MALPVTALLLPLALLLHAARPEQKLISEEDLQVQLVESGGGVVQPGRSLRL |
| | hinge-CD8 TM- | SCAASGFTLSGYGMHWVRQAPGKGLEWVSLISYDGSNKYYADSVKGRFTI |
| | CD3ζ | SRDDSKNTLYLRIVINSLRAEDTAVYYCARGRNDFWSGYYTAGMDVWGQG |
| | | TTVTVSSGGGGSGGGGSGGGGSDIQMTQSPSSLSASVGDRVTITCQASQGIR |
| | | KYLNWYQQKPGKVPKLLIYDASNLETGVPSRFSGSGSGTDFTFAISSLQPED |
| | | TATYYCQQYDDFPFTFGQGTRLEIKRTTTPAPRPPTPAPTIASQPLSLRPEAC |
| | | RPAAGGAVHTRGLDFACDIYIWAPLAGTCGVLLLSLVITLYCRVKFSRSAD |
| | | APAYKQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHM |
| | | QALPPR |
| 31 | anti-CD4-CD8 | MALPVTALLLPLALLLHAARPYPYDVPDYAQVQLQQWGAGLLKPSETLSL |
| | hinge-CD8 TM-4- | TCAVYGGSFSGYYWSWIRQPPGKGLEWIGEINHSGSTNYNPSLKSRVTISV |
| | 1-BB | DTSKNQFSLKLSSVTAADTAVYYCARVINWFDPWGQGTLVTGGGGSGGG |
| | | GSGGGGSDIQMTQSPSSVSASVGDRVTITCRASQDISSWLAWYQHKPGKAP |
| | | KLLIYAASSLQSGVPSRFSGSGSGTDFTLTISSLQPEDFATYYCQQANSFPYT |
| | | FGQGTKLEIKTTTPAPRPPTPAPTIASQPLSLRPEACRPAAGGAVHTRGLDFA |
| | | CDIYIWAPLAGTCGVLLLSLVITLYCRSKRSRLLHSDYMNMTPRRPGPTRK |
| | | HYQPYAPPRDFAAYRSKRGRKKLLYIFKQPFMRPVQTTQEEDGCSCRFPEE |
| | | EEGGCEL |
| 32 | tandem antiCCR5- | MALPVTALLLPLALLLHAARPQVQLVESGGGVVQPGRSLRLSCAASGFTLS |
| | VRC01 CAR | GYGMHWVRQAPGKGLEWVSLISYDGSNKYYADSVKGRFTISRDDSKNTL |
| | | YLRMNSLRAEDTAVYYCARGRNDFWSGYYTAGMDVWGQGTTVTVSSGG |
| | | GGSGGGGSGGGGSDIQMTQSPSSLSASVGDRVTITCQASQGIRKYLNWYQ |
| | | QKPGKVPKLLIYDASNLETGVPSRFSGSGSGTDFTFAISSLQPEDTATYYCQ |
| | | QYDDFPFTFGQGTRLEIKRGGGGSGGGGSGGGGSQVQLVQSGGQMKKPGE |
| | | SMRISCRASGYEFIDCTLNWIRLAPGKRPEWMGWLKPRGGAVNYARPLQG |
| | | RVTMTRDVYSDTAFLELRSLTVDDTAVYFCTRGKNCDYNWDFEHWGRGT |
| | | PVIVSSGGGGSGGGGSGGGGSEIVLTQSPGTLSLSPGETAIISCRTSQYGSLA |
| | | WYQQRPGQAPRLVIYSGSTRAAGIPDRFSGSRWGPDYNLTISNLESGDFGV |
| | | YYCQQYEFFGQGTKVQVDIKTTTPAPRPPTPAPTIASQPLSLRPEACRPAAG |
| | | GAVHTRGLDFACDIYIWAPLAGTCGVLLLSLVITLYCKRGRKKLLYIFKQPF |
| | | MRPVQTTQEEDGCSCRFPEEEEGGCELRVKFSRSADAPAYKQGQNQLYNE |
| | | LNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 33 | tandem antiCD4- | MALPVTALLLPLALLLHAARPQVQLQQWGAGLLKPSETLSLTCAVYGGSF |
| | VRC01 CAR | SGYYWSWIRQPPGKGLEWIGEINHSGSTNYNPSLKSRVTISVDTSKNQFSLK |
| | | LSSVTAADTAVYYCARVINWFDPWGQGTLVTGGGGSGGGGSGGGGSDIQ |
| | | MTQSPSSVSASVGDRVTITCRASQDISSWLAWYQHKPGKAPKLLIYAASSL |
| | | QSGVPSRFSGSGSGTDFTLTISSLQPEDFATYYCQQANSFPYTFGQGTKLEIK |
| | | GGGGSGGGGSGGGGSQVQLVQSGGQMKKPGESMRISCRASGYEFIDCTLN |
| | | WIRLAPGKRPEWMGWLKPRGGAVNYARPLQGRVTMTRDVYSDTAFLELR |
| | | SLTVDDTAVYFCTRGKNCDYNWDFEHWGRGTPVIVSSGGGGSGGGGSGG |
| | | GGSEIVLTQSPGTLSLSPGETAIISCRTSQYGSLAWYQQRPGQAPRLVIYSGS |
| | | TRAAGIPDRFSGSRWGPDYNLTISNLESGDFGVYYCQQYEFFGQGTKVQVD |
| | | IKTTTPAPRPPTPAPTIASQPLSLRPEACRPAAGGAVHTRGLDFACDIYIWAP |
| | | LAGTCGVLLLSLVITLYCKRGRKKLLYIFKQPFMRPVQTTQEEDGCSCRFPE |
| | | EEEGGCELRVKFSRSADAPAYKQGQNQLYNELNLGRREEYDVLDKRRGR |
| | | DPEMGGKPRRKNPQEGLYNELQKDKMAEAYSEIGMKGERRRGKGHDGLY |
| | | QGLSTATKDTYDALHMQALPPR |
| 34 | IL-2 signal peptide | MYRMQLLSCIALSLALVTNS |
| 35 | CCR5 CAR-T No. | MALPVTALLLPLALLLHAARPQVTLKESGPTLVKPTQTLTLTCTLSGFSLST |
| | 1 | SGVSVGWIRQPPGKALEWLASINWNDDKCYSPSLKSRLTITKDTPKNQVVL |
| | | AMSNMDPADTATYSCALDMPPHDSGPQSFDASDVWGPGTMVTVSSGGGG |
| | | SGGGGSGGGGSSYELMQLPSVSVSPGQTASITCSGDNLGDKYACWYQQKP |
| | | GRSPVLVIYGDNKRPSGIPERFSGSNSGNTATLTISGTQAMDEADYYCQAW |
| | | DTSTAVFGTGTKLTVLTTTPAPRPPTPAPTIASQPLSLRPEACRPAAGGAVH |
| | | TRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLG |
| | | RREEYDVLDKRRGRDPEMGGKPRRKNPQEGLYNELQKDKMAEAYSEIGM |
| | | KGERRRGKGHDGLYQGLSTATKDTYDALHMQALPPR |
| 36 | CCR5 CAR-T No. | MALPVTALLLPLALLLHAARPSYELMQLPSVSVSPGQTASITCSGDNLGDK |
| | 2 | YACWYQQKPGRSPVLVIYGDNKRPSGIPERFSGSNSGNTATLTISGTQAMD |
| | | EADYYCQAWDTSTAVFGTGTKLTVLGGGGSGGGGSGGGGSQVTLKESGP |
| | | TLVKPTQTLTLTCTLSGFSLSTSGVSVGWIRQPPGKALEWLASINWNDDKC |
| | | YSPSLKSRLTITKDTPKNQVVLAMSNMDPADTATYSCALDMPPHDSGPQSF |
| | | DASDVWGPGTMVTVSSTTTPAPRPPTPAPTIASQPLSLRPEACRPAAGGAV |
| | | HTRGLDFACDIYIWAPLAGTCGVLLLSLVITLYCKRGRKKLLYIFKQPFMRP |
| | | VQTTQEEDGCSCRFPEEEEGGCELRVKFSRSADAPAYKQGQNQLYNELNL |
| | | GRREEYDVLDKRRGRDPEMGGKPRRKNPQEGLYNELQKDKMAEAYSEIG |
| | | MKGERRRGKGHDGLYQGLSTATKDTYDALHMQALPPR |
| 37 | CCR5 CAR-T No. | MALPVTALLLPLALLLHAARPQVQLQESGPGLVKPSETLSLTCTVSGGSIGH |
| | 3 | DYWSWIRQPPGEGLEWIGFIFFDGSTNYNPSLNGRVTISLDTSKNQLSLRLT |
| | | SVTAADTAVYFCARLKGAWLLSEPPYFSSDGMDVWGQGTTVTVPSGGGG |
| | | SGGGGSGGGGSNFMLTQPPSASGTPGQRVSISCSGSSSDIGSNTVNWYQQL |
| | | PGTAPKLLIYSNNQRPSGVPDRFSGFKSGTSASLVISGLQSEDEADYYCAAW |
| | | DESLNGVVFGGGPRTTTPAPRPPTPAPTIASQPLSLRPEACRPAAGGAVHTR |
| | | GLDFACDIYIWAPLAGTCGVLLLSLVITLYCKRGRKKLLYIFKQPFMRPVQT |
| | | TQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKG |
| | | ERRRGKGHDGLYQGLSTATKDTYDALHMQALPPR |
| 38 | CCR5 CAR-T No. | MALPVTALLLPLALLLHAARPNFMLTQPPSASGTPGQRVSISCSGSSSDIGS |
| | 4 | NTVNWYQQLPGTAPKLLIYSNNQRPSGVPDRFSGFKSGTSASLVISGLQSED |
| | | EADYYCAAWDESLNGVVFGGGPRGGGGSGGGGSGGGGSQVQLQESGPGL |
| | | VKPSETLSLTCTVSGGSIGHDYWSWIRQPPGEGLEWIGFIFFDGSTNYNPSL |
| | | NGRVTISLDTSKNQLSLRLTSVTAADTAVYFCARLKGAWLLSEPPYFSSDG |
| | | MDVWGQGTTVTVPSTTTPAPRPPTPAPTIASQPLSLRPEACRPAAGGAVHT |
| | | RGLDFACDIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMK |
| | | GERRRGKGHDGLYQGLSTATKDTYDALHMQALPPR |
| 39 | CCR5 CAR-T No. | MALPVTALLLPLALLLHAARPQVQLQQWGAGLLKSWGTLSLTCAVSGASF |
| | 5 | SGYYWSWIRQPPGKGLEWIGEINHRGSTTYNPSLDGRVTISLDTSTNQISLK |
| | | LTSMTAADTAVYYCARTVAGTSDYWGQGTLVTVSSGSASAPTGGGGSGG |
| | | GGSGGGGSKTTLTQSPAFMSATPGDKVSISCKASRDVDDDVNWYQQRPGE |
| | | APIFIIEDATTLVPGISPRFSGSGYGTDFTLTINNIDSEDAAYYFCLQHDNFPL |
| | | TFGGGTKVEIKTTTPAPRPPTPAPTIASQPLSLRPEACRPAAGGAVHTRGLDF |
| | | ACDIYIWAPLAGTCGVLLLSLVITLYCKRGRKKLLYIFKQPFMRPVQTTQEE |
| | | DGCSCRFPEEEEGGCELRVKFSRSADAPAYKQGQNQLYNELNLGRREEYD |
| | | VLDKRRGRDPEMGGKPRRKNPQEGLYNELQKDKMAEAYSEIGMKGERRR |
| | | GKGHDGLYQGLSTATKDTYDALHMQALPPR |
| 40 | CCR5 CAR-T No. | MALPVTALLLPLALLLHAARPKTTLTQSPAFMSATPGDKVSISCKASRDVD |
| | 6 | DDVNWYQQRPGEAPIFIIEDATTLVPGISPRFSGSGYGTDFTLTINNIDSEDA |
| | | AYYFCLQHDNFPLTFGGGTKVEIKGGGGSGGGGSGGGGSQVQLQQWGAG |
| | | LLKSWGTLSLTCAVSGASFSGYYWSWIRQPPGKGLEWIGEINHRGSTTYNP |
| | | SLDGRVTISLDTSTNQISLKLTSMTAADTAVYYCARTVAGTSDYWGQGTL |
| | | VTVSSGSASAPTTTTPAPRPPTPAPTIASQPLSLRPEACRPAAGGAVHTRGLD |
| | | FACDIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRREEY |
| | | DVLDKRRGRDPEMGGKPRRKNPQEGLYNELQKDKMAEAYSEIGMKGERR |
| | | RGKGHDGLYQGLSTATKDTYDALHMQALPPR |
| 41 | CCR5 CAR-T No. | MALPVTALLLPLALLLHAARPQVTLKESGPTLVKPTQTLTLTCTFSGFSLRT |
| | 7 | TGEGVGWVRQPPGKALEWLALIYWDDDKRYSPSLKSRLTITKDTSKKQVV |
| | | LTMTNVDPADTATYYCTHEQYYYDTSGQPYYFDFWGQGTLVTVSSGGGG |
| | | SGGGGSGGGGSNIQVTQSPSSLSASVGDRVTMTCRASQDIRKNLNWYQQK |
| | | PGKAPKVLIYDASDLETGIPSRFSGSGSGTDFILTISSLQPEDIATYYCQQSDY |
| | | LPLTFGGGTKVDIKTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGE |
| | | RRRGKGHDGLYQGLSTATKDTYDALHMQALPPR |
| 42 | CCR5 CAR-T No. | MALPVTALLLPLALLLHAARPNIQVTQSPSSLSASVGDRVTMTCRASQDIR |
| | 8 | KNLNWYQQKPGKAPKVLIYDASDLETGIPSRFSGSGSGTDFILTISSLQPEDI |
| | | ATYYCQQSDYLPLTFGGGTKVDIKGGGGSGGGGSGGGGSQVTLKESGPTL |
| | | VKPTQTLTLTCTFSGFSLRTTGEGVGWVRQPPGKALEWLALIYWDDDKRY |
| | | SPSLKSRLTITKDTSKKQVVLTMTNVDPADTATYYCTHEQYYYDTSGQPY |
| | | YFDFWGQGTLVTVSSTTTPAPRPPTPAPTIASQPLSLRPEACRPAAGGAVHT |
| | | RGLDFACDIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMK |
| | | GERRRGKGHDGLYQGLSTATKDTYDALHMQALPPR |
| 43 | CCR5 CAR-T No. | MALPVTALLLPLALLLHAARPLVATATGVHSQVQLQQPGAGRVRPGASVK |
| | 9 | LSCKASGYSFTSYWMNWVKQRPGQGLEWIGMIHPSDSETRLNQKFNDRAT |
| | | LTVDKYSSTAYIQLSSPTSEDSAVYYCARGEYYYGIFDGGGGSGGGGSGGG |
| | | GSASLSASVGETVTITCRASENIYSYLAWYQQKQGKSPQLLVYNAKTLTEG |
| | | VPSRFSGSGSGTQFSLKINSLQPEDFGNYFCQHHYDTPRTFGGTTTPAPRPPT |
| | | PAPTIASQPLSLRPEACRPAAGGAVHTRGLDFACDIYIWAPLAGTCGVLLLS |
| | | LVITLYCKRGRKKLLYIFKQPFMRPVQTTQEEDGCSCRFPEEEEGGCELRVK |
| | | FSRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDTY |
| | | DALHMQALPPR |
| 44 | CCR5 CAR-T No. | MALPVTALLLPLALLLHAARPASLSASVGETVTITCRASENIYSYLAWYQQ |
| | 10 | KQGKSPQLLVYNAKTLTEGVPSRFSGSGSGTQFSLKINSLQPEDFGNYFCQH |
| | | HYDTPRTFGGGGGGSGGGGSGGGGSLVATATGVHSQVQLQQPGAGRVRP |
| | | GASVKLSCKASGYSFTSYWMNWVKQRPGQGLEWIGMIHPSDSETRLNQKF |
| | | NDRATLTVDKYSSTAYIQLSSPTSEDSAVYYCARGEYYYGIFDTTTPAPRPP |
| | | TPAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELRV |
| | | KFSRSADAPAYKQGQNQLYNELNLGRREEYDVLDKRRGRDPEMGGKPRR |
| | | KNPQEGLYNELQKDKMAEAYSEIGMKGERRRGKGHDGLYQGLSTATKDT |
| | | YDALHMQALPPR |
| 45 | CCR5 CAR-T No. | MALPVTALLLPLALLLHAARPEVQLVESGGGLVKPGGSLRLSCAASGYTFS |
| | 11 | NYWIGWVRQAPGKGLEWIGDIYPGGNYIRNNEKFKDKTTLSADTSKNTAY |
| | | LQMNSLKTEDTAVYYCGSSFGSNYVFAWFTYWGQGTLVTVSSGGGGSGG |
| | | GGSGGGGSDIVMTQSPLSLPVTPGEPASISCRSSQRLLSSYGHTYLHWYLQK |
| | | PGQSPQLLIYEVSNRFSGVPDRFSGSGSGTDFTLKISRVEAEDVGVYYCSQS |
| | | THVPLTFGQGTKVEIKTTTPAPRPPTPAPTIASQPLSLRPEACRPAAGGAVHT |
| | | RGLDFACDIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMK |
| | | GERRRGKGHDGLYQGLSTATKDTYDALHMQALPPR |
| 46 | CCR5 CAR-T No. | MALPVTALLLPLALLLHAARPDIVMTQSPLSLPVTPGEPASISCRSSQRLLSS |
| | 12 | YGHTYLHWYLQKPGQSPQLLIYEVSNRFSGVPDRFSGSGSGTDFTLKISRVE |
| | | AEDVGVYYCSQSTHVPLTFGQGTKVEIKGGGGSGGGGSGGGGSEVQLVES |
| | | GGGLVKPGGSLRLSCAASGYTFSNYWIGWVRQAPGKGLEWIGDIYPGGNY |
| | | IRNNEKFKDKTTLSADTSKNTAYLQMNSLKTEDTAVYYCGSSFGSNYVFA |
| | | WFTYWGQGTLVTVSSTTTPAPRPPTPAPTIASQPLSLRPEACRPAAGGAVHT |
| | | RGLDFACDIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMK |
| | | GERRRGKGHDGLYQGLSTATKDTYDALHMQALPPR |
| 47 | CCR5 CAR-T No. | MALPVTALLLPLALLLHAARPQVQLVESGGGVVQPGRSLRLSCAASGFTLS |
| | 13 | GYGMHWVRQAPGKGLEWVSLISYDGSNKYYADSVKGRFTISRDDSKNTL |
| | | YLRMNSLRAEDTAVYYCARGRNDFWSGYYTAGMDVWGQGTTVTVSSGG |
| | | GGSGGGGSGGGGSDIQMTQSPSSLSASVGDRVTITCQASQGIRKYLNWYQ |
| | | QKPGKVPKLLIYDASNLETGVPSRFSGSGSGTDFTFAISSLQPEDTATYYCQ |
| | | QYDDFPFTFGQGTRLEIKRTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYSE |
| | | IGMKGERRRGKGHDGLYQGLSTATKDTYDALHMQALPPR |
| 48 | CCR5 CAR-T No. | MALPVTALLLPLALLLHAARPDIQMTQSPSSLSASVGDRVTITCQASQGIRK |
| | 14 | YLNWYQQKPGKVPKLLIYDASNLETGVPSRFSGSGSGTDFTFAISSLQPEDT |
| | | ATYYCQQYDDFPFTFGQGTRLEIKRGGGGSGGGGSGGGGSQVQLVESGGG |
| | | VVQPGRSLRLSCAASGFTLSGYGMHWVRQAPGKGLEWVSLISYDGSNKY |
| | | YADSVKGRFTISRDDSKNTLYLRMNSLRAEDTAVYYCARGRNDFWSGYY |
| | | TAGMDVWGQGTTVTVSSTTTPAPRPPTPAPTIASQPLSLRPEACRPAAGGA |
| | | VHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYSE |
| | | IGMKGERRRGKGHDGLYQGLSTATKDTYDALHMQALPPR |
| 49 | CD4 CAR-T No. 1 | MALPVTALLLPLALLLHAARPEVQLVESGGGLVQPGRSLRLSCAASGFTFS |
| | | NYGMAWVRQAPGKGLEWVATISYDGSITYYRDSVKGRFTISRDNSKNTLY |
| | | LQMNSLRAEDTAVYYCAREEQYSSWYFDFWGQGTLVTVSSGGGGSGGGG |
| | | SGGGGSDIQLTQSPSSLSASVGDRVTITCRASQSVSISSHDLMQWYQQKPGK |
| | | APKLLIYDAFNLASGVPSRFSGSGSGTDFTLTISSLQPEDFATYYCQQSKDDP |
| | | YTFGQGTKLEIKTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 50 | CD4 CAR-T No. 2 | MALPVTALLLPLALLLHAARPDIQLTQSPSSLSASVGDRVTITCRASQSVSIS |
| | | SHDLMQWYQQKPGKAPKLLIYDAFNLASGVPSRFSGSGSGTDFTLTISSLQP |
| | | EDFATYYCQQSKDDPYTFGQGTKLEIKGGGGSGGGGSGGGGSEVQLVESG |
| | | GGLVQPGRSLRLSCAASGFTFSNYGMAWVRQAPGKGLEWVATISYDGSIT |
| | | YYRDSVKGRFTISRDNSKNTLYLQMNSLRAEDTAVYYCAREEQYSSWYFD |
| | | FWGQGTLVTVSSTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 51 | CD4 CAR-T No. 3 | MALPVTALLLPLALLLHAARPEVQLVESGGGLVQPGRSLRLSCAASGFTFS |
| | | NYGMAWVRQAPGKGLEWVATISYDGSITYYRDSVKGRFTISRDNSKNTLY |
| | | LQMNSLRAEDTAVYYCTREEQYSSWYFDFWGQGILVTVSSGGGGSGGGG |
| | | SGGGGSDIQLTQSPSSLSASVGDRVTITCRASQSVSISSHDLMQWYQQKPGK |
| | | APKLLIYDAFNLASGVPSRFSGSGSGTDFTLTISSLQPEDFATYYCQQSKDDP |
| | | YTFGQGTKLEIKTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 52 | CD4 CAR-T No. 4 | MALPVTALLLPLALLLHAARPDIQLTQSPSSLSASVGDRVTITCRASQSVSIS |
| | | SHDLMQWYQQKPGKAPKLLIYDAFNLASGVPSRFSGSGSGTDFTLTISSLQP |
| | | EDFATYYCQQSKDDPYTFGQGTKLEIKGGGGSGGGGSGGGGSEVQLVESG |
| | | GGLVQPGRSLRLSCAASGFTFSNYGMAWVRQAPGKGLEWVATISYDGSIT |
| | | YYRDSVKGRFTISRDNSKNTLYLQMNSLRAEDTAVYYCTREEQYSSWYFD |
| | | FWGQGILVTVSSTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 53 | CD4 CAR-T No. 5 | MALPVTALLLPLALLLHAARPEVQLVESGGGLVQPGRSLRLSCAASGFTFS |
| | | NYGMAWVRQAPGKGLEWVATISYDGSITYYRDSVKGRFTISRDNSKNTLY |
| | | LQMNSLRAEDTATYYCTREEQYSSWYFDFWGQGILVTVSSGGGGSGGGGS |
| | | GGGGSDIQLTQSPSSLSASVGDRVTITCRASQSVSISSHDLMQWYQQKPGK |
| | | APKLLIYDAFNLASGVPSRFSGSGSGTDFTLTISSLQPEDFATYYCQQSKDDP |
| | | YTFGQGTKLEIKTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 54 | CD4 CAR-T No. 6 | MALPVTALLLPLALLLHAARPDIQLTQSPSSLSASVGDRVTITCRASQSVSIS |
| | | SHDLMQWYQQKPGKAPKLLIYDAFNLASGVPSRFSGSGSGTDFTLTISSLQP |
| | | EDFATYYCQQSKDDPYTFGQGTKLEIKGGGGSGGGGSGGGGSEVQLVESG |
| | | GGLVQPGRSLRLSCAASGFTFSNYGMAWVRQAPGKGLEWVATISYDGSIT |
| | | YYRDSVKGRFTISRDNSKNTLYLQMNSLRAEDTATYYCTREEQYSSWYFD |
| | | FWGQGILVTVSSTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 55 | CD4 CAR-T No. 7 | MALPVTALLLPLALLLHAARPEVQLVESGGGLVQPGRSMRLSCAASGFTFS |
| | | NYGMAWVRQAPGKGLEWVATISYDGSITYYRDSVKGRFTISRDNSKNTLY |
| | | LQMNSLRAEDTATYYCTREEQYSSWYFDFWGQGILVTVSSGGGGSGGGGS |
| | | GGGGSDIQLTQSPSSLSASVGDRVTITCRASQSVSISSHDLMQWYQQKPGK |
| | | APKLLIYDAFNLASGVPSRFSGSGSGTDFTLTISSLQPEDFATYYCQQSKDDP |
| | | YTFGQGTKLEIKTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 56 | CD4 CAR-T No. 8 | MALPVTALLLPLALLLHAARPDIQLTQSPSSLSASVGDRVTITCRASQSVSIS |
| | | SHDLMQWYQQKPGKAPKLLIYDAFNLASGVPSRFSGSGSGTDFTLTISSLQP |
| | | EDFATYYCQQSKDDPYTFGQGTKLEIKGGGGSGGGGSGGGGSEVQLVESG |
| | | GGLVQPGRSMRLSCAASGFTFSNYGMAWVRQAPGKGLEWVATISYDGSIT |
| | | YYRDSVKGRFTISRDNSKNTLYLQMNSLRAEDTATYYCTREEQYSSWYFD |
| | | FWGQGILVTVSSTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 57 | CD4 CAR-T No. 9 | MALPVTALLLPLALLLHAARPEVQLVESGGGLVQPGRSMRLSCAASGFTFS |
| | | NYGMAWVRQAPGKGLEWVATISYDGSITYYRDSVKGRFTISRDNSKNTLY |
| | | LQMNSLRAEDTATYYCTREEQYSSWYFDFWGQGILVTVSSGGGGSGGGGS |
| | | GGGGSDIQLTQSPSSLSVSLGDRATITCRASQSVSISSHDLMQWYQQKPGK |
| | | QPKLLIYDAFNLASGIPSRFSGSGSGTDFTLTISSVQPEDFATYYCQQSKDDP |
| | | YTFGQGTKLEIKTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 58 | CD4 CAR-T No. | MALPVTALLLPLALLLHAARPDIQLTQSPSSLSVSLGDRATITCRASQSVSIS |
| | 10 | SHDLMQWYQQKPGKQPKLLIYDAFNLASGIPSRFSGSGSGTDFTLTISSVQP |
| | | EDFATYYCQQSKDDPYTFGQGTKLEIKGGGGSGGGGSGGGGSEVQLVESG |
| | | GGLVQPGRSMRLSCAASGFTFSNYGMAWVRQAPGKGLEWVATISYDGSIT |
| | | YYRDSVKGRFTISRDNSKNTLYLQMNSLRAEDTATYYCTREEQYSSWYFD |
| | | FWGQGILVTVSSTTTPAPRPPTPAPTIASQPLSLRPEACRPAAGGAVHTRGL |
| | | DFACDIYIWAPLAGTCGVLLLSLVITLYCKRGRKKLLYIFKQPFMRPVQTTQ |
| | | EEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGER |
| | | RRGKGHDGLYQGLSTATKDTYDALHMQALPPR |
| 59 | CD4 CAR-T No. | MALPVTALLLPLALLLHAARPQVQLQQSGPEVVKPGASVKMSCKASGYTF |
| | 11 | TSYVIHWVRQKPGQGLDWIGYINPYNDGTDYDEKFKGKATLTSDTSTSTA |
| | | YMELSSLRSEDTAVYYCAREKDNYATGAWFAYWGQGTLVTVSSAGGGGS |
| | | GGGGSGGGGSDIVMTQSPDSLAVSLGERVTMNCKSSQSLLYSTNQKNYLA |
| | | WYQQKPGQSPKLLIYWASTRESGVPDRFSGSGSGTDFTLTISSVQAEDVAV |
| | | YYCQQYYSYRTFGGGTKLEIKRTTTPAPRPPTPAPTIASQPLSLRPEACRPAA |
| | | GGAVHTRGLDFACDIYIWAPLAGTCGVLLLSLVITLYCKRGRKKLLYIFKQ |
| | | PFMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 60 | CD4 CAR-T No. | MALPVTALLLPLALLLHAARPDIVMTQSPDSLAVSLGERVTMNCKSSQSLL |
| | 12 | YSTNQKNYLAWYQQKPGQSPKLLIYWASTRESGVPDRFSGSGSGTDFTLTI |
| | | SSVQAEDVAVYYCQQYYSYRTFGGGTKLEIKRGGGGSGGGGSGGGGSQV |
| | | QLQQSGPEVVKPGASVKMSCKASGYTFTSYVIHWVRQKPGQGLDWIGYIN |
| | | PYNDGTDYDEKFKGKATLTSDTSTSTAYMELSSLRSEDTAVYYCAREKDN |
| | | YATGAWFAYWGQGTLVTVSSATTTPAPRPPTPAPTIASQPLSLRPEACRPA |
| | | AGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQNQL |
| | | YNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 61 | CD4 CAR-T No. | MALPVTALLLPLALLLHAARPQVQLQQWGAGLLKPSETLSLTCAVYGGSF |
| | 13 | SGYYWSWIRQPPGKGLEWIGEINHSGSTNYNPSLKSRVTISVDTSKNQFSLK |
| | | LSSVTAADTAVYYCARVINWFDPWGQGTLVTGGGGSGGGGSGGGGSDIQ |
| | | MTQSPSSVSASVGDRVTITCRASQDISSWLAWYQHKPGKAPKLLIYAASSL |
| | | QSGVPSRFSGSGSGTDFTLTISSLQPEDFATYYCQQANSFPYTFGQGTKLEIK |
| | | TTTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEEE |
| | | EGGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPR |
| 62 | CD4 CAR-T No. | MALPVTALLLPLALLLHAARPDIQMTQSPSSVSASVGDRVTITCRASQDISS |
| | 14 | WLAWYQHKPGKAPKLLIYAASSLQSGVPSRFSGSGSGTDFTLTISSLQPEDF |
| | | ATYYCQQANSFPYTFGQGTKLEIKGGGGSGGGGSGGGGSQVQLQQWGAG |
| | | LLKPSETLSLTCAVYGGSFSGYYWSWIRQPPGKGLEWIGEINHSGSTNYNPS |
| | | LKSRVTISVDTSKNQFSLKLSSVTAADTAVYYCARVINWFDPWGQGTLVTT |
| | | TTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEEE |
| | | EGGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPR |
| 63 | CD4 CAR-T No. | MALPVTALLLPLALLLHAARPEEQLVESGGGLVKPGGSLRLSCAASGFSFS |
| | 15 | DCRMYWLRQAPGKGLEWIGVISVKSENYGANYAESVRGRFTISRDDSKNT |
| | | VYLQMNSLKTEDTAVYYCSASYYRYDVGAWFAYWGQGTLVTVSSAGGG |
| | | GSGGGGSGGGGSDIVMTQSPDSLAVSLGERATINCRASKSVSTSGYSYIYW |
| | | YQQKPGQPPKLLIYLASILESGVPDRFSGSGSGTDFTLTISSLQAEDVAVYYC |
| | | QHSRELPWTFGQGTKVEIKRTTTPAPRPPTPAPTIASQPLSLRPEACRPAAGG |
| | | AVHTRGLDFACDIYIWAPLAGTCGVLLLSLVITLYCKRGRKKLLYIFKQPF |
| | | MRPVQTTQEEDGCSCRFPEEEEGGCELRVKFSRSADAPAYKQGQNQLYNE |
| | | LNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 64 | CD4 CAR-T No. | MALPVTALLLPLALLLHAARPDIVMTQSPDSLAVSLGERATINCRASKSVST |
| | 16 | SGYSYIYWYQQKPGQPPKLLIYLASILESGVPDRFSGSGSGTDFTLTISSLQA |
| | | EDVAVYYCQHSRELPWTFGQGTKVEIKRGGGGSGGGGSGGGGSEEQLVES |
| | | GGGLVKPGGSLRLSCAASGFSFSDCRMYWLRQAPGKGLEWIGVISVKSEN |
| | | YGANYAESVRGRFTISRDDSKNTVYLQMNSLKTEDTAVYYCSASYYRYDV |
| | | GAWFAYWGQGTLVTVSSATTTPAPRPPTPAPTIASQPLSLRPEACRPAAGG |
| | | AVHTRGLDFACDIYIWAPLAGTCGVLLLSLVITLYCKRGRKKLLYIFKQPF |
| | | MRPVQTTQEEDGCSCRFPEEEEGGCELRVKFSRSADAPAYKQGQNQLYNE |
| | | LNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 65 | CXCR4 CAR-T | MALPVTALLLPLALLLHAARPQVQLQESGPGLVKPSETLSLTCTVSGGSISS |
| | No. 1 | SSYYWGWIRQPPGKGLEWIGSIYYSGSTYYNPSLKSRVTISVDTSKNQFSLK |
| | | LSSVTAADTAVYYCARHILTKAAGYWYFDLWGRGTLVTVSSGGGGSGGG |
| | | GSGGGGSQSVLIQPRSVSGSPGQSVTISCTGTSSDVGGYNYVSWYQQHPGK |
| | | APKLMIYDVSKRPSGVPDRFSGSKSGNTASLTISGLQAEDEADYYCCSYAG |
| | | SYRDYVFGTGTKLTVLTTTPAPRPPTPAPTIASQPLSLRPEACRPAAGGAVH |
| | | TRGLDFACDIYIWAPLAGTCGVLLLSLVITLYCKRGRKKLLYIFKQPFMRPV |
| | | QTTQEEDGCSCRFPEEEEGGCELRVKFSRSADAPAYKQGQNQLYNELNLG |
| | | RREEYDVLDKRRGRDPEMGGKPRRKNPQEGLYNELQKDKMAEAYSEIGM |
| | | KGERRRGKGHDGLYQGLSTATKDTYDALHMQALPPR |
| 66 | CXCR4 CAR-T | MALPVTALLLPLALLLHAARPQSVLIQPRSVSGSPGQSVTISCTGTSSDVGG |
| | No. 2 | YNYVSWYQQHPGKAPKLMIYDVSKRPSGVPDRFSGSKSGNTASLTISGLQA |
| | | EDEADYYCCSYAGSYRDYVFGTGTKLTVLGGGGSGGGGSGGGGSQVQLQ |
| | | ESGPGLVKPSETLSLTCTVSGGSISSSSYYWGWIRQPPGKGLEWIGSIYYSGS |
| | | TYYNPSLKSRVTISVDTSKNQFSLKLSSVTAADTAVYYCARHILTKAAGYW |
| | | YFDLWGRGTLVTVSSTTTPAPRPPTPAPTIASQPLSLRPEACRPAAGGAVHT |
| | | RGLDFACDIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMK |
| | | GERRRGKGHDGLYQGLSTATKDTYDALHMQALPPR |
| 67 | CXCR4 CAR-T | MALPVTALLLPLALLLHAARPQVQLQQWGAGLLKPSETLSLTCAVYGGSF |
| | No. 3 | SGYYWSWIRQPPGKGLEWIGEINHSGSTNYNPSLKSRVTISVDTSKNQFSLK |
| | | LSSVTAADTAVYYCARGRRSIAARPFSFDIWGQGTMVTVSSGGGGSGGGG |
| | | SGGGGSQPVLTQSPSASGTPGQRVTISCSGSSSNIGINPVNWYQQLPGAAPK |
| | | LLIYTTNQRPSGVPDRFSASKSGTSASPAISGLQSADEADYYCAAWDDRLN |
| | | GVVFGGGTKLTVLTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGE |
| | | RRRGKGHDGLYQGLSTATKDTYDALHMQALPPR |
| 68 | CXCR4 CAR-T | MALPVTALLLPLALLLHAARPQPVLTQSPSASGTPGQRVTISCSGSSSNIGIN |
| | No. 4 | PVNWYQQLPGAAPKLLIYTTNQRPSGVPDRFSASKSGTSASPAISGLQSADE |
| | | ADYYCAAWDDRLNGVVFGGGTKLTVLGGGGSGGGGSGGGGSQVQLQQ |
| | | WGAGLLKPSETLSLTCAVYGGSFSGYYWSWIRQPPGKGLEWIGEINHSGST |
| | | NYNPSLKSRVTISVDTSKNQFSLKLSSVTAADTAVYYCARGRRSIAARPFSF |
| | | DIWGQGTMVTVSSTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTT |
| | | QEEDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGMKGE |
| | | RRRGKGHDGLYQGLSTATKDTYDALHMQALPPR |
| 69 | CXCR4 CAR-T | MALPVTALLLPLALLLHAARPEVQLVESGGGLVQPGGSLRLSCAAAGFTFS |
| | No. 5 | SYSMNWVRQAPGKGLEWVSYISSRSRTIYYADSVKGRFTISRDNAKNSLYL |
| | | QMNSLRDEDTAVYYCARDYGGQPPYYYYYGMDVWGQGTTVTVSSAGGG |
| | | GSGGGGSGGGGSDIQMTQSPSSLSASVGDRVTITCRASQGISSWLAWYQQK |
| | | PEKAPKSLIYAASSLQSGVPSRFSGSGSGTDFTLTISSLQPEDFVTYYCQQYN |
| | | SYPRTFGQGTKVEIKRTTTPAPRPPTPAPTIASQPLSLRPEACRPAAGGAVHT |
| | | RGLDFACDIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYNELNLGR |
| | | REEYDVLDKRRGRDPEMGGKPRRKNPQEGLYNELQKDKMAEAYSEIGMK |
| | | GERRRGKGHDGLYQGLSTATKDTYDALHMQALPPR |
| 70 | CXCR4 CAR-T | MALPVTALLLPLALLLHAARPDIQMTQSPSSLSASVGDRVTITCRASQGISS |
| | No. 6 | WLAWYQQKPEKAPKSLIYAASSLQSGVPSRFSGSGSGTDFTLTISSLQPEDF |
| | | VTYYCQQYNSYPRTFGQGTKVEIKRGGGGSGGGGSGGGGSEVQLVESGGG |
| | | LVQPGGSLRLSCAAAGFTFSSYSMNWVRQAPGKGLEWVSYISSRSRTIYYA |
| | | DSVKGRFTISRDNAKNSLYLQMNSLRDEDTAVYYCARDYGGQPPYYYYY |
| | | GMDVWGQGTTVTVSSATTTPAPRPPTPAPTIASQPLSLRPEACRPAAGGAV |
| | | HTRGLDFACDIYIWAPLAGTCGVLLLSLVITLYCKRGRKKLLYIFKQPFMRP |
| | | VQTTQEEDGCSCRFPEEEEGGCELRVKFSRSADAPAYKQGQNQLYNELNL |
| | | GRREEYDVLDKRRGRDPEMGGKPRRKNPQEGLYNELQKDKMAEAYSEIG |
| | | MKGERRRGKGHDGLYQGLSTATKDTYDALHMQALPPR |
| 71 | CXCR4 CAR-T | MALPVTALLLPLALLLHAARPQVQLVQSGAEVKKPGASVKVSCKASGYTF |
| | No. 7 | TSYGISWVRQAPGQGLEWMGWISAYNGNTNYAQKLQGRVTMTTDTSTST |
| | | AYMELRSLRSDDTAVYYCARDTPGIAARRYYYYGMDVWGQGTTVTVSSG |
| | | GGGSGGGGSGGGGSSSELTQDPAVSVALGQTVRITCQGDSLRKFFASWYQ |
| | | QKPGQAPVLVIYGKNSRPSGIPDRFSGSNSRNTASLTITGAQAEDEGDYYCN |
| | | SRDSRDNHQVFGAGTKVTVLSTTTPAPRPPTPAPTIASQPLSLRPEACRPAA |
| | | GGAVHTRGLDFACDIYIWAPLAGTCGVLLLSLVITLYCKRGRKKLLYIFKQ |
| | | PFMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 72 | CXCR4 CAR-T | MALPVTALLLPLALLLHAARPSSELTQDPAVSVALGQTVRITCQGDSLRKF |
| | No. 8 | FASWYQQKPGQAPVLVIYGKNSRPSGIPDRFSGSNSRNTASLTITGAQAEDE |
| | | GDYYCNSRDSRDNHQVFGAGTKVTVLSGGGGSGGGGSGGGGSQVQLVQS |
| | | GAEVKKPGASVKVSCKASGYTFTSYGISWVRQAPGQGLEWMGWISAYNG |
| | | NTNYAQKLQGRVTMTTDTSTSTAYMELRSLRSDDTAVYYCARDTPGIAAR |
| | | RYYYYGMDVWGQGTTVTVSSTTTPAPRPPTPAPTIASQPLSLRPEACRPAA |
| | | GGAVHTRGLDFACDIYIWAPLAGTCGVLLLSLVITLYCKRGRKKLLYIFKQ |
| | | PFMRPVQTTQEEDGCSCRFPEEEEGGCELRVKFSRSADAPAYKQGQNQLY |
| | | NELNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 73 | CD4 eTCR No. 11 | MQSGTHWRVLGLCLLSVGVWGQQVQLQQSGPEVVKPGASVKMSCKASG |
| | | YTFTSYVIHWVRQKPGQGLDWIGYINPYNDGTDYDEKFKGKATLTSDTSTS |
| | | TAYMELSSLRSEDTAVYYCAREKDNYATGAWFAYWGQGTLVTVSSAGGG |
| | | GSGGGGSGGGGSDIVMTQSPDSLAVSLGERVTMNCKSSQSLLYSTNQKNY |
| | | LAWYQQKPGQSPKLLIYWASTRESGVPDRFSGSGSGTDFTLTISSVQAEDV |
| | | AVYYCQQYYSYRTFGGGTKLEIKRGGGGSGGGGSGGGGSDGNEEMGGIT |
| | | QTPYKVSISGTTVILTCPQYPGSEILWQHNDKNIGGDEDDKNIGSDEDHLSL |
| | | KEFSELEQSGYYVCYPRGSKPEDANFYLYLRARVCENCMEMDVMSVATIV |
| | | IVDICITGGLLLLVYYWSKNRKAKAKPVTRGAGAGGRQRGQNKERPPPVP |
| | | NPDYEPIRKGQRDLYSGLNQRRIGSGATNFSLLKQAGDVEENPGPMNYPLT |
| | | LEMDLENLEDLFWELDRLDNYNDTSLVENHLCPATEGPLMASFKAVFVPV |
| | | AYSLIFLLGVIGNVLVLVILERHRQTRSSTETFLFHLAVADLLLVFILPFAVA |
| | | EGSVGWVLGTFLCKTVIALHKVNFYCSSLLLACIAVDRYLAIVHAVHAYR |
| | | HRRLLSIHITCGTIWLVGFLLALPEILFAKVSQGHHNNSLPRCTFSQENQAET |
| | | HAWFTSRFLYHVAGFLLPMLVMGWCYVGVVHRLRQAQRRPQRQKAVRV |
| | | AILVTSIFFLCWSPYHIVIFLDTLARLKAVDNTCKLNGSLPVAITMCEFLGLA |
| | | HCCLNPMLYTFAGVKFRSDLSRLLTKLGCTGPASLCQLFPGWRRSSLSESE |
| | | NATSLTTF |
| 74 | CCR5 eTCR 807 | MQSGTHWRVLGLCLLSVGVWGQQVQLKQSGAELVRPGASVKLSCKTSGY |
| | | IFTNYWIHWVKQRSGQGLEWIARIYPGTGSNYYNEKLKDKATLTTDKSSST |
| | | VYIQLSSLKSEDSAVYFCAREGDYYGYGAMDYWGQGTSVTVSSGGGGSG |
| | | GGGSGGGGSDIVLTQSPASLVVSLGQRATISCRASKSVSTSGYYYMHWYQ |
| | | QKPGQPPKLLIYLASNLESGVPARFSGSGSGTDFTLNIHPVEEEDAATYYCH |
| | | HSREFPYTFGGGTKLEIKGGGGSGGGGSGGGGSDGNEEMGGITQTPYKVSI |
| | | SGTTVILTCPQYPGSEILWQHNDKNIGGDEDDKNIGSDEDHLSLKEFSELEQ |
| | | SGYYVCYPRGSKPEDANFYLYLRARVCENCMEMDVMSVATIVIVDICITGG |
| | | LLLLVYYWSKNRKAKAKPVTRGAGAGGRQRGQNKERPPPVPNPDYEPIRK |
| | | GQRDLYSGLNQRRIGSGATNFSLLKQAGDVEENPGPMNYPLTLEMDLENL |
| | | EDLFWELDRLDNYNDTSLVENHLCPATEGPLMASFKAVFVPVAYSLIFLLG |
| | | VIGNVLVLVILERHRQTRSSTETFLFHLAVADLLLVFILPFAVAEGSVGWVL |
| | | GTFLCKTVIALHKVNFYCSSLLLACIAVDRYLAIVHAVHAYRHRRLLSIHIT |
| | | CGTIWLVGFLLALPEILFAKVSQGHHNNSLPRCTFSQENQAETHAWFTSRFL |
| | | YHVAGFLLPMLVMGWCYVGVVHRLRQAQRRPQRQKAVRVAILVTSIFFL |
| | | CWSPYHIVIFLDTLARLKAVDNTCKLNGSLPVAITMCEFLGLAHCCLNPML |
| | | YTFAGVKFRSDLSRLLTKLGCTGPASLCQLFPGWRRSSLSESENATSLTTF |
| 75 | CCR5 eTCR 808 | MQSGTHWRVLGLCLLSVGVWGQQVQLQQSGPELVRPGASVKMSCEASGY |
| | | IFTSYWMHWVKQRPGQGLEWIGMIDPSNSETRLNQKFKDKATLTVDKSSN |
| | | TAYMQLSSLTSADSAVYYCARDYSYAMDYWGQGTSVTVSSGGGGSGGG |
| | | GSGGGGSDIKMTQSPSSMYASLGERVTITCKASQDISAYLSWFQQKPGKSP |
| | | KTLIYRANRLVDGVPPRFSGSGSGQDFSLTISSLGYEDMGFYYCLQYDEFPN |
| | | TFGGGTKLEITGGGGSGGGGSGGGGSDGNEEMGGITQTPYKVSISGTTVILT |
| | | CPQYPGSEILWQHNDKNIGGDEDDKNIGSDEDHLSLKEFSELEQSGYYVCY |
| | | PRGSKPEDANFYLYLRARVCENCMEMDVMSVATIVIVDICITGGLLLLVYY |
| | | WSKNRKAKAKPVTRGAGAGGRQRGQNKERPPPVPNPDYEPIRKGQRDLYS |
| | | GLNQRRIGSGATNFSLLKQAGDVEENPGPMNYPLTLEMDLENLEDLFWEL |
| | | DRLDNYNDTSLVENHLCPATEGPLMASFKAVFVPVAYSLIFLLGVIGNVLV |
| | | LVILERHRQTRSSTETFLFHLAVADLLLVFILPFAVAEGSVGWVLGTFLCKT |
| | | VIALHKVNFYCSSLLLACIAVDRYLAIVHAVHAYRHRRLLSIHITCGTIWLV |
| | | GFLLALPEILFAKVSQGHHNNSLPRCTFSQENQAETHAWFTSRFLYHVAGF |
| | | LLPMLVMGWCYVGVVHRLRQAQRRPQRQKAVRVAILVTSIFFLCWSPYHI |
| | | VIFLDTLARLKAVDNTCKLNGSLPVAITMCEFLGLAHCCLNPMLYTFAGVK |
| | | FRSDLSRLLTKLGCTGPASLCQLFPGWRRSSLSESENATSLTTF |
| 76 | CCR5 eTCR | MQSGTHWRVLGLCLLSVGVWGQEVQLVESGGGLVQPKGSLKLSCAASGF |
| | | TFNTYAMNWVRQAPGKGLEWVARIRNKSNNYATYYAASVKDRFTISRDD |
| | | SQSMLYLQMNNLKTEDTAMYYCVSLGEFAYWGQGTLVTVSAGGGGSGG |
| | | GGSGGGGSEIVLTQSPTTMAASPGEKVTITCSATSTINSNYLHWYQQKPGFS |
| | | PKLLIYRTSNLASGVPARFSGSGSGTSYSLTIGTMEAEDVATYYCQQGSTLP |
| | | FTFGSGTKLEIKGGGGSGGGGSGGGGSDGNEEMGGITQTPYKVSISGTTVIL |
| | | TCPQYPGSEILWQHNDKNIGGDEDDKNIGSDEDHLSLKEFSELEQSGYYVC |
| | | YPRGSKPEDANFYLYLRARVCENCMEMDVMSVATIVIVDICITGGLLLLVY |
| | | YWSKNRKAKAKPVTRGAGAGGRQRGQNKERPPPVPNPDYEPIRKGQRDL |
| | | YSGLNQRRIGSGATNFSLLKQAGDVEENPGPMNYPLTLEMDLENLEDLFW |
| | | ELDRLDNYNDTSLVENHLCPATEGPLMASFKAVFVPVAYSLIFLLGVIGNV |
| | | LVLVILERHRQTRSSTETFLFHLAVADLLLVFILPFAVAEGSVGWVLGTFLC |
| | | KTVIALHKVNFYCSSLLLACIAVDRYLAIVHAVHAYRHRRLLSIHITCGTIW |
| | | LVGFLLALPEILFAKVSQGHHNNSLPRCTFSQENQAETHAWFTSRFLYHVA |
| | | GFLLPMLVMGWCYVGVVHRLRQAQRRPQRQKAVRVAILVTSIFFLCWSPY |
| | | HIVIFLDTLARLKAVDNTCKLNGSLPVAITMCEFLGLAHCCLNPMLYTFAG |
| | | VKFRSDLSRLLTKLGCTGPASLCQLFPGWRRSSLSESENATSLTTF |
| 77 | Anti-CD19 CAR | MALPVTALLLPLALLLHAARPDIQMTQTTSSLSASLGDRVTISCRASQDISK |
| | | YLNWYQQKPDGTVKLLIYHTSRLHSGVPSRFSGSGSGTDYSLTISNLEQEDI |
| | | ATYFCQQGNTLPYTFGGGTKLEITGGGGSGGGGSGGGGSEVKLQESGPGL |
| | | VAPSQSLSVTCTVSGVSLPDYGVSWIRQPPRKGLEWLGVIWGSETTYYNSA |
| | | LKSRLTIIKDNSKSQVFLKMNSLQTDDTAIYYCAKHYYYGGSYAMDYWGQ |
| | | GTSVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQEEDG |
| | | CSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGK |
| | | GHDGLYQGLSTATKDTYDALHMQALPPR |
| 78 | ssCD4CCR5- | MALPVTALLLPLALLLHAARPEQKLISEEDLQVQLQQWGAGLLKPSETLSL |
| | CXCR5 | TCAVYGGSFSGYYWSWIRQPPGKGLEWIGEINHSGSTNYNPSLKSRVTISV |
| | | DTSKNQFSLKLSSVTAADTAVYYCARVINWFDPWGQGTLVTGGGGSGGG |
| | | GSGGGGSDIQMTQSPSSVSASVGDRVTITCRASQDISSWLAWYQHKPGKAP |
| | | KLLIYAASSLQSGVPSRFSGSGSGTDFTLTISSLQPEDFATYYCQQANSFPYT |
| | | FGQGTKLEIKTTTPAPRPPTPAPTIASQPLSLRPEACRPAAGGAVHTRGLDFA |
| | | CDIYIWAPLAGTCGVLLLSLVITLYCRVKFSRSADAPAYKQGQNQLYNELN |
| | | LGRREEYDVLDKRRGRDPEMGGKPRRKNPQEGLYNELQKDKMAEAYSEI |
| | | GMKGERRRGKGHDGLYQGLSTATKDTYDALHMQALPPRGSGATNFSLLK |
| | | QAGDVEENPGPMALPVTALLLPLALLLHAARPYPYDVPDYAQVQLVESGG |
| | | GVVQPGRSLRLSCAASGFTLSGYGMHWVRQAPGKGLEWVSLISYDGSNK |
| | | YYADSVKGRFTISRDDSKNTLYLRMNSLRAEDTAVYYCARGRNDFWSGY |
| | | YTAGMDVWGQGTTVTVSSGGGGSGGGGSGGGGSDIQMTQSPSSLSASVG |
| | | DRVTITCQASQGIRKYLNWYQQKPGKVPKLLIYDASNLETGVPSRFSGSGS |
| | | GTDFTFAISSLQPEDTATYYCQQYDDFPFTFGQGTRLEIKRTTTPAPRPPTPA |
| | | PTIASQPLSLRPEACRPAAGGAVHTRGLDFACDIYIWAPLAGTCGVLLLSLV |
| | | ITLYCRSKRSRLLHSDYMNMTPRRPGPTRKHYQPYAPPRDFAAYRSKRGR |
| | | KKLLYIFKQPFMRPVQTTQEEDGCSCRFPEEEEGGCELGSGATNFSLLKQA |
| | | GDVEENPGPMNYPLTLEMDLENLEDLFWELDRLDNYNDTSLVENHLCPAT |
| | | EGPLMASFKAVFVPVAYSLIFLLGVIGNVLVLVILERHRQTRSSTETFLFHLA |
| | | VADLLLVFILPFAVAEGSVGWVLGTFLCKTVIALHKVNFYCSSLLLACIAV |
| | | DRYLAIVHAVHAYRHRRLLSIHITCGTIWLVGFLLALPEILFAKVSQGHHNN |
| | | SLPRCTFSQENQAETHAWFTSRFLYHVAGFLLPMLVMGWCYVGVVHRLR |
| | | QAQRRPQRQKAVRVAILVTSIFFLCWSPYHIVIFLDTLARLKAVDNTCKLN |
| | | GSLPVAITMCEFLGLAHCCLNPMLYTFAGVKFRSDLSRLLTKLGCTGPASL |
| | | CQLFPGWRRSSLSESENATSLTTF |
| 79 | ssCCR5CD4- | MALPVTALLLPLALLLHAARPEQKLISEEDLQVQLVESGGGVVQPGRSLRL |
| | CXCR5 | SCAASGFTLSGYGMHWVRQAPGKGLEWVSLISYDGSNKYYADSVKGRFTI |
| | | SRDDSKNTLYLRMNSLRAEDTAVYYCARGRNDFWSGYYTAGMDVWGQG |
| | | TTVTVSSGGGGSGGGGSGGGGSDIQMTQSPSSLSASVGDRVTITCQASQGIR |
| | | KYLNWYQQKPGKVPKLLIYDASNLETGVPSRFSGSGSGTDFTFAISSLQPED |
| | | TATYYCQQYDDFPFTFGQGTRLEIKRTTTPAPRPPTPAPTIASQPLSLRPEAC |
| | | RPAAGGAVHTRGLDFACDIYIWAPLAGTCGVLLLSLVITLYCRVKFSRSAD |
| | | APAYKQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHM |
| | | QALPPRGSGATNFSLLKQAGDVEENPGPMALPVTALLLPLALLLHAARPYP |
| | | YDVPDYAQVQLQQWGAGLLKPSETLSLTCAVYGGSFSGYYWSWIRQPPG |
| | | KGLEWIGEINHSGSTNYNPSLKSRVTISVDTSKNQFSLKLSSVTAADTAVYY |
| | | CARVINWFDPWGQGTLVTGGGGSGGGGSGGGGSDIQMTQSPSSVSASVGD |
| | | RVTITCRASQDISSWLAWYQHKPGKAPKLLIYAASSLQSGVPSRFSGSGSGT |
| | | DFTLTISSLQPEDFATYYCQQANSFPYTFGQGTKLEIKTTTPAPRPPTPAPTIA |
| | | SQPLSLRPEACRPAAGGAVHTRGLDFACDIYIWAPLAGTCGVLLLSLVITLY |
| | | CRSKRSRLLHSDYMNMTPRRPGPTRKHYQPYAPPRDFAAYRSKRGRKKLL |
| | | YIFKQPFMRPVQTTQEEDGCSCRFPEEEEGGCELGSGATNFSLLKQAGDVE |
| | | ENPGPMNYPLTLEMDLENLEDLFWELDRLDNYNDTSLVENHLCPATEGPL |
| | | MASFKAVFVPVAYSLIFLLGVIGNVLVLVILERHRQTRSSTETFLFHLAVAD |
| | | LLLVFILPFAVAEGSVGWVLGTFLCKTVIALHKVNFYCSSLLLACIAVDRYL |
| | | AIVHAVHAYRHRRLLSIHITCGTIWLVGFLLALPEILFAKVSQGHHNNSLPR |
| | | CTFSQENQAETHAWFTSRFLYHVAGFLLPMLVMGWCYVGVVHRLRQAQR |
| | | RPQRQKAVRVAILVTSIFFLCWSPYHIVIFLDTLARLKAVDNTCKLNGSLPV |
| | | AITMCEFLGLAHCCLNPMLYTFAGVKFRSDLSRLLTKLGCTGPASLCQLFP |
| | | GWRRSSLSESENATSLTTF |

What is claimed is:

1. An engineered immune cell comprising:
a) a chimeric receptor (CR) comprising: i) a CR antigen binding domain specifically recognizing a CR target antigen; ii) a CR transmembrane domain, and iii) an intracellular CR signaling domain; and
b) a chimeric co-receptor (CCOR) comprising: i) a CCOR antigen binding domain specifically recognizing a CCOR target antigen; ii) a CCOR transmembrane domain; and iii) an intracellular CCOR co-stimulatory domain,
wherein the CR target antigen is CCR5 or CXCR4 and the CCOR target antigen is CD4, or wherein the CR target antigen is CD4 and the CCOR target antigen is CCR5 or CXCR4.

2. An engineered immune cell comprising:
a chimeric receptor (CR) comprising: i) a CR antigen binding domain specifically recognizing a CR target antigen; ii) a CR transmembrane domain, and iii) an intracellular CR signaling domain,
wherein the CR target antigen is selected from the group consisting of CCR5, CXCR4 and CD4.

3. The engineered immune cell of claim 1 or 2, wherein the CCR5, CXCR4 or CD4 is in tandem with a broadly neutralizing antibody.

4. The engineered immune cell of claim 3, the broadly neutralizing antibody is VRC01, PGT121, 3BNC117 or 10-1074.

5. The engineered immune cell of any one of claims 1-4, further comprising one or more co-receptors (“COR”).

6. An engineered immune cell comprising:
a) a first nucleic acid encoding a chimeric receptor (CR), wherein the CR comprises: i) a CR antigen binding domain specifically recognizing a CR target antigen; ii) a CR transmembrane domain, and iii) an intracellular CR signaling domain; and
b) second nucleic acid encoding a chimeric co-receptor (CCOR), wherein the CCOR comprises: i) a CCOR antigen binding domain specifically recognizing a CCOR target antigen; ii) a CCOR transmembrane domain; and iii) an intracellular CCOR co-stimulatory signaling domain;
wherein the CR target antigen is CCR5 or CXCR4 and the CCOR target antigen is CD4, or wherein the CR target antigen is CD4 and the CCOR target antigen is CCR5 or CXCR4.

7. An engineered immune cell comprising:
a nucleic acid encoding a chimeric receptor (CR), wherein the CR comprises: i) a CR antigen binding domain specifically recognizing a CR target antigen; ii) a CR transmembrane domain, and iii) an intracellular CR signaling domain,
wherein the CR target antigen is selected from the group consisting of CCR5, CXCR4 and CD4.

8. The engineered immune cell of claim 6 or claim 7, wherein the CCR5, CXCR4 or CD4 is in tandem with a broadly neutralizing antibody.

9. The engineered immune cell of claim 8, the broadly neutralizing antibody is VRC01, PGT121, 3BNC117, 10-1074.

10. The engineered immune cell of claim 6-9, further comprising one or more nucleic acid(s) encoding one or more co-receptors (“COR”).

11. The engineered immune cell of any one of claims 1-10, wherein the CR is a chimeric antigen receptor (“CAR”).

12. The engineered immune cell of claim 11, wherein the CR transmembrane is derived from CD8α.

13. The engineered immune cell of claim 11, wherein the intracellular CR signaling domain is derived from CD3ζ.

14. The engineered immune cell of any one of claims 11-13, wherein the CR further comprises an intracellular CR co-stimulatory domain.

15. The engineered immune cell of claim 14, wherein the intracellular CR co-stimulatory signaling domain comprises a cytoplasmic domain of 4-1-BB.

16. The engineered immune cell of any one of claims 1-10, wherein the CR does not comprise an intracellular co-stimulatory domain.

17. The engineered immune cell of any one of claims 1-10, wherein the CR is a chimeric T cell receptor (“cTCR”).

18. The engineered immune cell of claim 17, wherein the CR transmembrane domain is derived from the transmembrane domain of a TCR subunit selected from the group consisting of TCRα, TCRβ, TCRγ, TCRδ, CD3γ, CD3ε, and CD3δ.

19. The engineered immune cell of claim 18, wherein the CR transmembrane domain is derived from the transmembrane domain of CD3ε.

20. The engineered immune cell of any one of claims 17-19, wherein the intracellular CR signaling domain is derived from the intracellular signaling domain of a TCR subunit selected from the group consisting of TCRα, TCRβ, TCRγ, TCRδ, CD3γ, CD3ε, and CD3δ.

21. The engineered immune cell of claim 20, wherein the intracellular CR signaling domain is derived from the intracellular signaling domain of CD3ε.

22. The engineered immune cell of any one of claims 17-21, wherein the CR transmembrane domain and intracellular CR signaling domain are derived from the same or different TCR subunit(s).

23. The engineered immune cell of any one of claims 17-22, wherein the CR further comprises a portion of an extracellular domain of a TCR subunit.

24. The engineered immune cell of any one of claims 17-23, wherein the CR comprises the CR antigen binding domain fused to the N-terminus of CD3ε.

25. The engineered immune cell of any one of claims 1-24, wherein the CR target antigen is CD4.

26. The engineered immune cell of any one of claims 1, 5, 6, and 10-25, wherein the CCOR target antigen is CD4.

27. The engineered immune cell of any one of claims 1, 5, 6, and 10-24 and 26, wherein the CR target antigen is CCR5 or CXCR4 and the CCOR target antigen is CD4.

28. The engineered immune cell of any one of claims 1, 5, 6, and 10-25, wherein the CR target antigen is CD4 and the CCOR target antigen is CCR5 or CXCR4.

29. The engineered immune cell of claim 25-28, wherein the CR antigen binding domain or the CCOR antigen binding domain specifically recognizes domain 1 of CD4 (CD4 D1).

30. The engineered immune cell of any one of claims 5 and 10-29, wherein the one or more COR is selected from the group consisting of CXCR5, α4β7, and CCR9.

31. The engineered immune cell of claim 30, wherein the one or more COR comprises both α4β7 and CCR9.

32. The engineered immune cell of any one of claims 1-31, wherein the engineered immune cell is modified to reduce or eliminate expression of CCR5 within the cell.

33. The engineered immune cell of any one of claims 1-32, wherein the engineered immune cell is modified to express an anti-HIV antibody.

34. The engineered immune cell of claim 33, wherein the anti-HIV antibody is a broadly neutralizing antibody.

35. The engineered immune cell of claim 34, wherein the broadly neutralizing antibody is VRC01, PGT121, 3BNC117 10-1074.

36. The engineered immune cell of any one of claims 1-35, wherein the CR antigen binding domain is scFv or sdAb.

37. The engineered immune cell of any one of claims 1-36, wherein the CCOR antigen binding domain is scFv or sdAb.

38. The engineered immune cell of any one of claims 1-37, wherein the engineered immune cell is selected from the group consisting of a cytotoxic T cell, a helper T cell, a natural killer cell, a γδT cell and a natural killer T cell.

39. A pharmaceutical composition comprising the engineered immune cell of any one of claims 1-37 and a pharmaceutically acceptable carrier.

40. The pharmaceutical composition of claim 39, wherein the pharmaceutical composition comprises at least two different types of engineered immune cells according to any one of claims 1-38.

41. A method of treating an infectious disease in an individual, comprising administering to the individual an effective amount of a pharmaceutical composition of claim 39 or 40.

42. The method of claim 41, wherein the infectious disease is an infection by a virus selected from the group consisting of HIV and HTLV.

43. The method of claim 42, wherein the infectious disease is HIV.

44. The method of claim 41-43, further comprising administering to the individual a second anti-infectious agent.

45. A method of treating a cancer in an individual, comprising administering to the individual an effective amount of a pharmaceutical composition of claim 39 or 40.

46. The method of claim 45, wherein the cancer is T cell lymphoma.

47. The method of claim 45 or 46, further comprising administering to the individual a second anti-cancer agent.

48. The method of any one of claims 41-47, wherein the individual is a human.

49. A method of making an engineered immune cell of any one of claims 1-38, comprising:
a) providing a population of immune cells;
b) introducing into the population of immune cells a first nucleic acid encoding the CR.

50. The method of claim49, further comprising:
c) introducing into the population of immune cells a second nucleic acid encoding the CCOR.

51. The method of claim50, further comprising introducing into the population of immune cells one or more nucleic acids encoding one or more CORs.

52. The method of any one of claims 49-51, wherein the first nucleic acid, the second nucleic acid, and/or the COR encoding nucleic acids are introduced into the cell via a viral vector.

53. The method of any one of claims 49-52, further comprising introducing into the population of immune cells a nucleic acid encoding a broadly neutralizing antibody (bNAb) or a HIV fusion inhibition peptide

54. The method of any one of claims 49-53, further comprising inactivating the CCR5 gene in the cell.

55. The method of claim 54, wherein the CCR5 gene is inactivated by using the method selected from the group consisting of: CRISPR/Cas9, TALEN, ZFN, siRNA, and antisense RNA.

56. The method of any one of claims 49-55, further comprising obtaining the population of immune cells from the peripheral blood of an individual.

57. The method of claim 56, wherein the population of immune cells are further enriched for CD4+ cells.

58. The method of claim 56 or 57, wherein the population of immune cells are further enriched for CD8+ cells.


SEQUENCE LISTING (79 sequences)

SEQ ID NO: 1 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 2 - protein, 45 aa, Homo sapiens
TTTPAPRPPTPAPTIASQPLSLRPEACRPAAGGAVHTRGLDFACD

SEQ ID NO: 3 - protein, 24 aa, Homo sapiens
IYIWAPLAGTCGVLLLSLVITLYC

SEQ ID NO: 4 - protein, 42 aa, Homo sapiens
KRGRKKLLYIFKQPFMRPVQTTQEEDGCSCRFPEEEEGGCEL

SEQ ID NO: 5 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 7 - protein, 22 aa, Homo sapiens
MQSGTHWRVLGLCLLSVGVWGQ

SEQ ID NO: 8 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 9 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 10 - protein, 239 aa, artificial sequence
  VRC01 scFv
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 11 - protein, 478 aa, artificial sequence
  anti-CD4 CAR
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 492 aa, artificial sequence
  anti-CCR5 CAR
MALPVTALLLPLALLLHAARPQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 741 aa, artificial sequence
  tandem antiCD4-antiCCR5 CAR
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 872 aa, artificial sequence
  anti-CD4 CAR with CXCR5
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 15 - protein, 886 aa, artificial sequence
  anti-CCR5 CAR with CXCR5
MALPVTALLLPLALLLHAARPQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 16 - protein, 1135 aa, artificial sequence
  tandem antiCD4-antiCCR5 CAR with CXCR5
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTF

SEQ ID NO: 17 - protein, 1159 aa, artificial sequence
  anti-CD4 CAR with CXCR5 and VRC01
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTFGSGATNFSLLKQAGDVEENPGPMYRMQLLSCIALSLALVTNSQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKHHHHHH

SEQ ID NO: 18 - protein, 1173 aa, artificial sequence
  anti-CCR5 CAR with CXCR5 and VRC01
MALPVTALLLPLALLLHAARPQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTFGSGATNFSLLKQAGDVEENPGPMYRMQLLSCIALSLALVTNSQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKHHHHHH

SEQ ID NO: 19 - protein, 1422 aa, artificial sequence
  tandem antiCD4-antiCCR5 CAR with CXCR5 and VRC01
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTFGSGATNFSLLKQAGDVEENPGPMYRMQLLSCIALSLALVTNSQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKHHHHHH

SEQ ID NO: 20 - protein, 456 aa, artificial sequence
  anti-CD4 eTCR
MQSGTHWRVLGLCLLSVGVWGQQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 21 - protein, 462 aa, artificial sequence
  anti-CCR5 eTCR
MQSGTHWRVLGLCLLSVGVWGQEVQLVESGGGLVQPKGSLKLSCAASGFTFNTYAMNWVRQAPGKGLEWVARIRNKSNNYATYYAASVKDRFTISRDDSQSMLYLQMNNLKTEDTAMYYCVSLGEFAYWGQGTLVTVSAGGGGSGGGGSGGGGSEIVLTQSPTTMAASPGEKVTITCSATSTINSNYLHWYQQKPGFSPKLLIYRTSNLASGVPARFSGSGSGTSYSLTIGTMEAEDVATYYCQQGSTLPFTFGSGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 22 - protein, 850 aa, artificial sequence
  anti-CD4 eTCR with CXCR5
MQSGTHWRVLGLCLLSVGVWGQQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTF

SEQ ID NO: 23 - protein, 864 aa, artificial sequence
  anti-CCR5 eTCR with CXCR5
MQSGTHWRVLGLCLLSVGVWGQQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTF

SEQ ID NO: 24 - protein, 1113 aa, artificial sequence
  tandem antiCD4-anti-CCR5 eTCR with CXCR5
MQSGTHWRVLGLCLLSVGVWGQQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 25 - protein, 1400 aa, artificial sequence
  tandem antiCD4-anti-CCR5 eTCR with CXCR5 and VRC01
MQSGTHWRVLGLCLLSVGVWGQQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTFGSGATNFSLLKQAGDVEENPGPMYRMQLLSCIALSLALVTNSQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKHHHHHH

SEQ ID NO: 26 - protein, 898 aa, artificial sequence
  split signal antiCD4-antiCCR5 CAR
MALPVTALLLPLALLLHAARPEQKLISEEDLQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPYPYDVPDYAQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 27 - protein, 446 aa, artificial sequence
  anti-CD4-CD8 hinge-CD8 TM-CD3
MALPVTALLLPLALLLHAARPEQKLISEEDLQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 430 aa, artificial sequence
  anti-CCR5-CD8 hinge-CD8 TM-4-1-BB
MALPVTALLLPLALLLHAARPYPYDVPDYAQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 29 - protein, 898 aa, artificial sequence
  split signal antiCCR5-antiCD4 CAR
MALPVTALLLPLALLLHAARPEQKLISEEDLQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPYPYDVPDYAQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 30 - protein, 460 aa, artificial sequence
  anti-CCR5-CD8 hinge-CD8 TM-CD3
MALPVTALLLPLALLLHAARPEQKLISEEDLQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 416 aa, artificial sequence
  anti-CD4-CD8 hinge-CD8 TM-4-1-BB
MALPVTALLLPLALLLHAARPYPYDVPDYAQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 32 - protein, 746 aa, artificial sequence
  tandem antiCCR5-VRC01 CAR
MALPVTALLLPLALLLHAARPQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRGGGGSGGGGSGGGGSQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 732 aa, artificial sequence
  tandem antiCD4-VRC01 CAR
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 20 aa, Homo sapiens
MYRMQLLSCIALSLALVTNS

SEQ ID NO: 35 - protein, 492 aa, artificial sequence
  CCR5 CAR-T No. 1
MALPVTALLLPLALLLHAARPQVTLKESGPTLVKPTQTLTLTCTLSGFSLSTSGVSVGWIRQPPGKALEWLASINWNDDKCYSPSLKSRLTITKDTPKNQVVLAMSNMDPADTATYSCALDMPPHDSGPQSFDASDVWGPGTMVTVSSGGGGSGGGGSGGGGSSYELMQLPSVSVSPGQTASITCSGDNLGDKYACWYQQKPGRSPVLVIYGDNKRPSGIPERFSGSNSGNTATLTISGTQAMDEADYYCQAWDTSTAVFGTGTKLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 492 aa, artificial sequence
  CCR5 CAR-T No. 2
MALPVTALLLPLALLLHAARPSYELMQLPSVSVSPGQTASITCSGDNLGDKYACWYQQKPGRSPVLVIYGDNKRPSGIPERFSGSNSGNTATLTISGTQAMDEADYYCQAWDTSTAVFGTGTKLTVLGGGGSGGGGSGGGGSQVTLKESGPTLVKPTQTLTLTCTLSGFSLSTSGVSVGWIRQPPGKALEWLASINWNDDKCYSPSLKSRLTITKDTPKNQVVLAMSNMDPADTATYSCALDMPPHDSGPQSFDASDVWGPGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 493 aa, artificial sequence
  CCR5 CAR-T No. 3
MALPVTALLLPLALLLHAARPQVQLQESGPGLVKPSETLSLTCTVSGGSIGHDYWSWIRQPPGEGLEWIGFIFFDGSTNYNPSLNGRVTISLDTSKNQLSLRLTSVTAADTAVYFCARLKGAWLLSEPPYFSSDGMDVWGQGTTVTVPSGGGGSGGGGSGGGGSNFMLTQPPSASGTPGQRVSISCSGSSSDIGSNTVNWYQQLPGTAPKLLIYSNNQRPSGVPDRFSGFKSGTSASLVISGLQSEDEADYYCAAWDESLNGVVFGGGPRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 493 aa, artificial sequence
  CCR5 CAR-T No. 4
MALPVTALLLPLALLLHAARPNFMLTQPPSASGTPGQRVSISCSGSSSDIGSNTVNWYQQLPGTAPKLLIYSNNQRPSGVPDRFSGFKSGTSASLVISGLQSEDEADYYCAAWDESLNGVVFGGGPRGGGGSGGGGSGGGGSQVQLQESGPGLVKPSETLSLTCTVSGGSIGHDYWSWIRQPPGEGLEWIGFIFFDGSTNYNPSLNGRVTISLDTSKNQLSLRLTSVTAADTAVYFCARLKGAWLLSEPPYFSSDGMDVWGQGTTVTVPSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 489 aa, artificial sequence
  CCR5 CAR-T No. 5
MALPVTALLLPLALLLHAARPQVQLQQWGAGLLKSWGTLSLTCAVSGASFSGYYWSWIRQPPGKGLEWIGEINHRGSTTYNPSLDGRVTISLDTSTNQISLKLTSMTAADTAVYYCARTVAGTSDYWGQGTLVTVSSGSASAPTGGGGSGGGGSGGGGSKTTLTQSPAFMSATPGDKVSISCKASRDVDDDVNWYQQRPGEAPIFIIEDATTLVPGISPRFSGSGYGTDFTLTINNIDSEDAAYYFCLQHDNFPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 489 aa, artificial sequence
  CCR5 CAR-T No. 6
MALPVTALLLPLALLLHAARPKTTLTQSPAFMSATPGDKVSISCKASRDVDDDVNWYQQRPGEAPIFIIEDATTLVPGISPRFSGSGYGTDFTLTINNIDSEDAAYYFCLQHDNFPLTFGGGTKVEIKGGGGSGGGGSGGGGSQVQLQQWGAGLLKSWGTLSLTCAVSGASFSGYYWSWIRQPPGKGLEWIGEINHRGSTTYNPSLDGRVTISLDTSTNQISLKLTSMTAADTAVYYCARTVAGTSDYWGQGTLVTVSSGSASAPTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 492 aa, artificial sequence
  CCR5 CAR-T No. 7
MALPVTALLLPLALLLHAARPQVTLKESGPTLVKPTQTLTLTCTFSGFSLRTTGEGVGWVRQPPGKALEWLALIYWDDDKRYSPSLKSRLTITKDTSKKQVVLTMTNVDPADTATYYCTHEQYYYDTSGQPYYFDFWGQGTLVTVSSGGGGSGGGGSGGGGSNIQVTQSPSSLSASVGDRVTMTCRASQDIRKNLNWYQQKPGKAPKVLIYDASDLETGIPSRFSGSGSGTDFILTISSLQPEDIATYYCQQSDYLPLTFGGGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 492 aa, artificial sequence
  CCR5 CAR-T No. 8
MALPVTALLLPLALLLHAARPNIQVTQSPSSLSASVGDRVTMTCRASQDIRKNLNWYQQKPGKAPKVLIYDASDLETGIPSRFSGSGSGTDFILTISSLQPEDIATYYCQQSDYLPLTFGGGTKVDIKGGGGSGGGGSGGGGSQVTLKESGPTLVKPTQTLTLTCTFSGFSLRTTGEGVGWVRQPPGKALEWLALIYWDDDKRYSPSLKSRLTITKDTSKKQVVLTMTNVDPADTATYYCTHEQYYYDTSGQPYYFDFWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 468 aa, artificial sequence
  CCR5 CAR-T No. 9
MALPVTALLLPLALLLHAARPLVATATGVHSQVQLQQPGAGRVRPGASVKLSCKASGYSFTSYWMNWVKQRPGQGLEWIGMIHPSDSETRLNQKFNDRATLTVDKYSSTAYIQLSSPTSEDSAVYYCARGEYYYGIFDGGGGSGGGGSGGGGSASLSASVGETVTITCRASENIYSYLAWYQQKQGKSPQLLVYNAKTLTEGVPSRFSGSGSGTQFSLKINSLQPEDFGNYFCQHHYDTPRTFGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 468 aa, artificial sequence
  CCR5 CAR-T No. 10
MALPVTALLLPLALLLHAARPASLSASVGETVTITCRASENIYSYLAWYQQKQGKSPQLLVYNAKTLTEGVPSRFSGSGSGTQFSLKINSLQPEDFGNYFCQHHYDTPRTFGGGGGGSGGGGSGGGGSLVATATGVHSQVQLQQPGAGRVRPGASVKLSCKASGYSFTSYWMNWVKQRPGQGLEWIGMIHPSDSETRLNQKFNDRATLTVDKYSSTAYIQLSSPTSEDSAVYYCARGEYYYGIFDTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 493 aa, artificial sequence
  CCR5 CAR-T No. 11
MALPVTALLLPLALLLHAARPEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSGGGGSGGGGSGGGGSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 493 aa, artificial sequence
  CCR5 CAR-T No. 12
MALPVTALLLPLALLLHAARPDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKGGGGSGGGGSGGGGSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 492 aa, artificial sequence; identical to SEQ ID NO: 12
  CCR5 CAR-T No. 13

SEQ ID NO: 48 - protein, 492 aa, artificial sequence
  CCR5 CAR-T No. 14
MALPVTALLLPLALLLHAARPDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRGGGGSGGGGSGGGGSQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 490 aa, artificial sequence
  CD4 CAR-T No. 1
MALPVTALLLPLALLLHAARPEVQLVESGGGLVQPGRSLRLSCAASGFTFSNYGMAWVRQAPGKGLEWVATISYDGSITYYRDSVKGRFTISRDNSKNTLYLQMNSLRAEDTAVYYCAREEQYSSWYFDFWGQGTLVTVSSGGGGSGGGGSGGGGSDIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 490 aa, artificial sequence
  CD4 CAR-T No. 2
MALPVTALLLPLALLLHAARPDIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIKGGGGSGGGGSGGGGSEVQLVESGGGLVQPGRSLRLSCAASGFTFSNYGMAWVRQAPGKGLEWVATISYDGSITYYRDSVKGRFTISRDNSKNTLYLQMNSLRAEDTAVYYCAREEQYSSWYFDFWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 490 aa, artificial sequence
  CD4 CAR-T No. 3
MALPVTALLLPLALLLHAARPEVQLVESGGGLVQPGRSLRLSCAASGFTFSNYGMAWVRQAPGKGLEWVATISYDGSITYYRDSVKGRFTISRDNSKNTLYLQMNSLRAEDTAVYYCTREEQYSSWYFDFWGQGILVTVSSGGGGSGGGGSGGGGSDIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 490 aa, artificial sequence
  CD4 CAR-T No. 4
MALPVTALLLPLALLLHAARPDIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIKGGGGSGGGGSGGGGSEVQLVESGGGLVQPGRSLRLSCAASGFTFSNYGMAWVRQAPGKGLEWVATISYDGSITYYRDSVKGRFTISRDNSKNTLYLQMNSLRAEDTAVYYCTREEQYSSWYFDFWGQGILVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 490 aa, artificial sequence
  CD4 CAR-T No. 5
MALPVTALLLPLALLLHAARPEVQLVESGGGLVQPGRSLRLSCAASGFTFSNYGMAWVRQAPGKGLEWVATISYDGSITYYRDSVKGRFTISRDNSKNTLYLQMNSLRAEDTATYYCTREEQYSSWYFDFWGQGILVTVSSGGGGSGGGGSGGGGSDIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 490 aa, artificial sequence
  CD4 CAR-T No. 6
MALPVTALLLPLALLLHAARPDIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIKGGGGSGGGGSGGGGSEVQLVESGGGLVQPGRSLRLSCAASGFTFSNYGMAWVRQAPGKGLEWVATISYDGSITYYRDSVKGRFTISRDNSKNTLYLQMNSLRAEDTATYYCTREEQYSSWYFDFWGQGILVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 490 aa, artificial sequence
  CD4 CAR-T No. 7
MALPVTALLLPLALLLHAARPEVQLVESGGGLVQPGRSMRLSCAASGFTFSNYGMAWVRQAPGKGLEWVATISYDGSITYYRDSVKGRFTISRDNSKNTLYLQMNSLRAEDTATYYCTREEQYSSWYFDFWGQGILVTVSSGGGGSGGGGSGGGGSDIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 490 aa, artificial sequence
  CD4 CAR-T No. 8
MALPVTALLLPLALLLHAARPDIQLTQSPSSLSASVGDRVTITCRASQSVSISSHDLMQWYQQKPGKAPKLLIYDAFNLASGVPSRFSGSGSGTDFTLTISSLQPEDFATYYCQQSKDDPYTFGQGTKLEIKGGGGSGGGGSGGGGSEVQLVESGGGLVQPGRSMRLSCAASGFTFSNYGMAWVRQAPGKGLEWVATISYDGSITYYRDSVKGRFTISRDNSKNTLYLQMNSLRAEDTATYYCTREEQYSSWYFDFWGQGILVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 490 aa, artificial sequence
  CD4 CAR-T No. 9
MALPVTALLLPLALLLHAARPEVQLVESGGGLVQPGRSMRLSCAASGFTFSNYGMAWVRQAPGKGLEWVATISYDGSITYYRDSVKGRFTISRDNSKNTLYLQMNSLRAEDTATYYCTREEQYSSWYFDFWGQGILVTVSSGGGGSGGGGSGGGGSDIQLTQSPSSLSVSLGDRATITCRASQSVSISSHDLMQWYQQKPGKQPKLLIYDAFNLASGIPSRFSGSGSGTDFTLTISSVQPEDFATYYCQQSKDDPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 490 aa, artificial sequence
  CD4 CAR-T No. 10
MALPVTALLLPLALLLHAARPDIQLTQSPSSLSVSLGDRATITCRASQSVSISSHDLMQWYQQKPGKQPKLLIYDAFNLASGIPSRFSGSGSGTDFTLTISSVQPEDFATYYCQQSKDDPYTFGQGTKLEIKGGGGSGGGGSGGGGSEVQLVESGGGLVQPGRSMRLSCAASGFTFSNYGMAWVRQAPGKGLEWVATISYDGSITYYRDSVKGRFTISRDNSKNTLYLQMNSLRAEDTATYYCTREEQYSSWYFDFWGQGILVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 495 aa, artificial sequence
  CD4 CAR-T No. 11
MALPVTALLLPLALLLHAARPQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSAGGGGSGGGGSGGGGSDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 495 aa, artificial sequence
  CD4 CAR-T No. 12
MALPVTALLLPLALLLHAARPDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRGGGGSGGGGSGGGGSQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 478 aa, artificial sequence; identical to SEQ ID NO: 11
  CD4 CAR-T No. 13

SEQ ID NO: 62 - protein, 478 aa, artificial sequence
  CD4 CAR-T No. 14
MALPVTALLLPLALLLHAARPDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 496 aa, artificial sequence
  CD4 CAR-T No. 15
MALPVTALLLPLALLLHAARPEEQLVESGGGLVKPGGSLRLSCAASGFSFSDCRMYWLRQAPGKGLEWIGVISVKSENYGANYAESVRGRFTISRDDSKNTVYLQMNSLKTEDTAVYYCSASYYRYDVGAWFAYWGQGTLVTVSSAGGGGSGGGGSGGGGSDIVMTQSPDSLAVSLGERATINCRASKSVSTSGYSYIYWYQQKPGQPPKLLIYLASILESGVPDRFSGSGSGTDFTLTISSLQAEDVAVYYCQHSRELPWTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 496 aa, artificial sequence
  CD4 CAR-T No. 16
MALPVTALLLPLALLLHAARPDIVMTQSPDSLAVSLGERATINCRASKSVSTSGYSYIYWYQQKPGQPPKLLIYLASILESGVPDRFSGSGSGTDFTLTISSLQAEDVAVYYCQHSRELPWTFGQGTKVEIKRGGGGSGGGGSGGGGSEEQLVESGGGLVKPGGSLRLSCAASGFSFSDCRMYWLRQAPGKGLEWIGVISVKSENYGANYAESVRGRFTISRDDSKNTVYLQMNSLKTEDTAVYYCSASYYRYDVGAWFAYWGQGTLVTVSSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 494 aa, artificial sequence
  CXCR4 CAR-T No. 1
MALPVTALLLPLALLLHAARPQVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARHILTKAAGYWYFDLWGRGTLVTVSSGGGGSGGGGSGGGGSQSVLIQPRSVSGSPGQSVTISCTGTSSDVGGYNYVSWYQQHPGKAPKLMIYDVSKRPSGVPDRFSGSKSGNTASLTISGLQAEDEADYYCCSYAGSYRDYVFGTGTKLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 494 aa, artificial sequence
  CXCR4 CAR-T No. 2
MALPVTALLLPLALLLHAARPQSVLIQPRSVSGSPGQSVTISCTGTSSDVGGYNYVSWYQQHPGKAPKLMIYDVSKRPSGVPDRFSGSKSGNTASLTISGLQAEDEADYYCCSYAGSYRDYVFGTGTKLTVLGGGGSGGGGSGGGGSQVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARHILTKAAGYWYFDLWGRGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 491 aa, artificial sequence
  CXCR4 CAR-T No. 3
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGRRSIAARPFSFDIWGQGTMVTVSSGGGGSGGGGSGGGGSQPVLTQSPSASGTPGQRVTISCSGSSSNIGINPVNWYQQLPGAAPKLLIYTTNQRPSGVPDRFSASKSGTSASPAISGLQSADEADYYCAAWDDRLNGVVFGGGTKLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 491 aa, artificial sequence
  CXCR4 CAR-T No. 4
MALPVTALLLPLALLLHAARPQPVLTQSPSASGTPGQRVTISCSGSSSNIGINPVNWYQQLPGAAPKLLIYTTNQRPSGVPDRFSASKSGTSASPAISGLQSADEADYYCAAWDDRLNGVVFGGGTKLTVLGGGGSGGGGSGGGGSQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGRRSIAARPFSFDIWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 493 aa, artificial sequence
  CXCR4 CAR-T No. 5
MALPVTALLLPLALLLHAARPEVQLVESGGGLVQPGGSLRLSCAAAGFTFSSYSMNWVRQAPGKGLEWVSYISSRSRTIYYADSVKGRFTISRDNAKNSLYLQMNSLRDEDTAVYYCARDYGGQPPYYYYYGMDVWGQGTTVTVSSAGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFVTYYCQQYNSYPRTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 493 aa, artificial sequence
  CXCR4 CAR-T No. 6
MALPVTALLLPLALLLHAARPDIQMTQSPSSLSASVGDRVTITCRASQGISSWLAWYQQKPEKAPKSLIYAASSLQSGVPSRFSGSGSGTDFTLTISSLQPEDFVTYYCQQYNSYPRTFGQGTKVEIKRGGGGSGGGGSGGGGSEVQLVESGGGLVQPGGSLRLSCAAAGFTFSSYSMNWVRQAPGKGLEWVSYISSRSRTIYYADSVKGRFTISRDNAKNSLYLQMNSLRDEDTAVYYCARDYGGQPPYYYYYGMDVWGQGTTVTVSSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 494 aa, artificial sequence
  CXCR4 CAR-T No. 7
MALPVTALLLPLALLLHAARPQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDTPGIAARRYYYYGMDVWGQGTTVTVSSGGGGSGGGGSGGGGSSSELTQDPAVSVALGQTVRITCQGDSLRKFFASWYQQKPGQAPVLVIYGKNSRPSGIPDRFSGSNSRNTASLTITGAQAEDEGDYYCNSRDSRDNHQVFGAGTKVTVLSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 494 aa, artificial sequence
  CXCR4 CAR-T No. 8
MALPVTALLLPLALLLHAARPSSELTQDPAVSVALGQTVRITCQGDSLRKFFASWYQQKPGQAPVLVIYGKNSRPSGIPDRFSGSNSRNTASLTITGAQAEDEGDYYCNSRDSRDNHQVFGAGTKVTVLSGGGGSGGGGSGGGGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDTPGIAARRYYYYGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 867 aa, artificial sequence
  CD4 eTCR No. 11
MQSGTHWRVLGLCLLSVGVWGQQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSAGGGGSGGGGSGGGGSDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTF

SEQ ID NO: 74 - protein, 863 aa, artificial sequence
  CCR5 eTCR 807
MQSGTHWRVLGLCLLSVGVWGQQVQLKQSGAELVRPGASVKLSCKTSGYIFTNYWIHWVKQRSGQGLEWIARIYPGTGSNYYNEKLKDKATLTTDKSSSTVYIQLSSLKSEDSAVYFCAREGDYYGYGAMDYWGQGTSVTVSSGGGGSGGGGSGGGGSDIVLTQSPASLVVSLGQRATISCRASKSVSTSGYYYMHWYQQKPGQPPKLLIYLASNLESGVPARFSGSGSGTDFTLNIHPVEEEDAATYYCHHSREFPYTFGGGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTF

SEQ ID NO: 75 - protein, 855 aa, artificial sequence
  CCR5 eTCR 808
MQSGTHWRVLGLCLLSVGVWGQQVQLQQSGPELVRPGASVKMSCEASGYIFTSYWMHWVKQRPGQGLEWIGMIDPSNSETRLNQKFKDKATLTVDKSSNTAYMQLSSLTSADSAVYYCARDYSYAMDYWGQGTSVTVSSGGGGSGGGGSGGGGSDIKMTQSPSSMYASLGERVTITCKASQDISAYLSWFQQKPGKSPKTLIYRANRLVDGVPPRFSGSGSGQDFSLTISSLGYEDMGFYYCLQYDEFPNTFGGGTKLEITGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTF

SEQ ID NO: 76 - protein, 856 aa, artificial sequence
  CCR5 eTCR
MQSGTHWRVLGLCLLSVGVWGQEVQLVESGGGLVQPKGSLKLSCAASGFTFNTYAMNWVRQAPGKGLEWVARIRNKSNNYATYYAASVKDRFTISRDDSQSMLYLQMNNLKTEDTAMYYCVSLGEFAYWGQGTLVTVSAGGGGSGGGGSGGGGSEIVLTQSPTTMAASPGEKVTITCSATSTINSNYLHWYQQKPGFSPKLLIYRTSNLASGVPARFSGSGSGTSYSLTIGTMEAEDVATYYCQQGSTLPFTFGSGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTF

SEQ ID NO: 77 - protein, 486 aa, artificial sequence
  Anti-CD19 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 1292 aa, artificial sequence
  ssCD4CCR5-CXCR5
MALPVTALLLPLALLLHAARPEQKLISEEDLQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPYPYDVPDYAQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTF

SEQ ID NO: 79 - protein, 1292 aa, artificial sequence
  ssCCR5CD4-CXCR5
MALPVTALLLPLALLLHAARPEQKLISEEDLQVQLVESGGGVVQPGRSLRLSCAASGFTLSGYGMHWVRQAPGKGLEWVSLISYDGSNKYYADSVKGRFTISRDDSKNTLYLRMNSLRAEDTAVYYCARGRNDFWSGYYTAGMDVWGQGTTVTVSSGGGGSGGGGSGGGGSDIQMTQSPSSLSASVGDRVTITCQASQGIRKYLNWYQQKPGKVPKLLIYDASNLETGVPSRFSGSGSGTDFTFAISSLQPEDTATYYCQQYDDFPFTFGQGTRLEIKRTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPYPYDVPDYAQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELGSGATNFSLLKQAGDVEENPGPMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPGWRRSSLSESENATSLTTF